SEQUENCE LISTING (4 sequences)

SEQ ID NO: 1 - DNA, 20 bp, synthetic construct
  misc_feature 1..20: note = synthetic nucleotide
  source 1..20: mol_type = other DNA
ccaacaacccttggtcctgt

SEQ ID NO: 2 - DNA, 20 bp, synthetic construct
  misc_feature 1..20: note = synthetic nucleotide
  source 1..20: mol_type = other DNA
gggtagcctggaaatagggc

SEQ ID NO: 3 - DNA, 997 bp, Homo sapiens
  source 1..997: mol_type = genomic DNA
agtcctgactacggcctccgggccctttgtccccgctagcggcgctcggggtgggggagccaggaggggcgggagacgggcgggtatgggccgcgcgggcgcaggctcccccgggcgccgcaggcagcggtgccagagccggggcaggcggcggccgcgagcccctcggcggcggaaggccccagcgtgcaggcgcaggagggcgcggcgccggcggaagaagccctgtccccgcagcttgcgaccggagatccacgaatgtcccaagtcccaggacccgtgcgcgtcccgggacgactggcgctgtgcgcgctccatgcacgagttttccgccaaggacatcgacgggcacatggttaacctggacaagtaccggggcttcgtgtgcatcgtcaccaacgtggcctcccagtgaggcaagaccgaagtaaactacactcagctcgtcgacctgcacgcccgatacgctgagtgtggtttgcggatcctggccttcccgtgtaaccagttcgggaagcaggagccagggagtaacgaagagatcaaagagttcgccgcgggctacaacgtcaaattcgatatgttcagcaagatctgcgtgaacggggacgacgcccacccgctgtggaagtggatgaagatccaacccaagggcaagggcatcctgggaaatgccatcaagtggaacttcaccaagttcctcatcgacaagaacggctgcgtggtgaagcgctacggacccatggaggagcccctggtgatagagaaggacctgccccactatttctagctccacaagtgtgtggccccgcccgagcccctgcccacgcccttggagccttccaccggcactcatgacggcctgcctgcaaacctgctggtggggcagacccgaaaatccagcgtgcaccccgccggaggaaggtcccatggcctgctgggcttggctcggcgcccccacccctggctaccttgtgggaataaacagacaaattag

SEQ ID NO: 4 - DNA, 979 bp, synthetic construct
  misc_feature 1..979: note = Synthetic nucleic acid
  source 1..979: mol_type = other DNA
agtcctgactacggcctccgggccctttgtccccgctagcggcgctcggggtgggggagccaggaggggcgggagacgggcgggtatgggccgcgcgggcgcaggctcccccgggcgccgcaggcagcggtgccagagccggggcaggcggcggccgcgagcccctcggcggcggaaggccccagcgtgcaggcgcaggagggcgcggcgccggcggaagaagccctgtccccgcagcttgcgaccggagatccacgaatgtcccaagtcccaggacccgtgcgcgtcccgggacgactggcgctgtgcgcgctccatgcacgagttttccgccaaggacatcgacgggcacatggttaacctggacaagtaccggggcttcgtgtgcatcgtcaccaacgtggcctcccagtgaggcaagaccgaagtaaactacactcagctcgtcgacctgcacgcccgatacgctgagtgtggtttgcggatcctggccttcccgtgtaaccagttcgggaagcaggagccagggagtaacgaagagatcaaagagttcgccgcgggctacaacgtcaaattcgatatgttcagcaagatctgcgtgaacggggacgacgcccacccgctgtggaagtggatgaagatccaacccaagggcaagggcatcctgggaaatgccatcaagtggaacttcaccaagttcctcatcgacaagaacggctgcgtggtgaagcgctacggacccatggaggagcccctggtgatagagaaggacctgccccactatttctagctccacaagtgtgtggccccgcccgagcccctgcccacgcccttggagccttccaccggcactcatgacggcctgcctgcaaacctgctggtggggcagacccgaaaatccagcgtgcaccccgccggaggaaggtcccatggcctgctgggcttggctcggcgcccccacccctggctaccttgtggg

(19) United States
(12) Patent Application Publication
(10) Pub. No.: US 2025/0001009 A1
(43) Pub. Date: Jan. 2, 2025

(54) THERAPY FOR PROGRAMMED CELL DEATH IN A SUBJECT TRANSPLANTED WITH A DONOR LIVER CARRYING THE PNPLA3 RS738409:G VARIANT

(71) Applicants: University of Pittsburgh - Of the Commonwealth System of Higher Education, Pittsburgh, PA (US); Regents of the University of Michigan, Ann Arbor, MI (US); Kyushu University, National University Corporation, Fukuoka-shi (JP)

(72) Inventors: Alejandro Soto-Gutierrez, Pittsburgh, PA (US); Kazuki Takeishi, Fukuoka-shi (JP); Kazutoyo Morita, Fukuoka-shi (JP); Nils Haep, Berlin (DE); Rodrigo Machado Florentino, Pittsburgh, PA (US); Abhinav Achreja, Ann Arbor, MI (US); Deepak Nagrath, Ann Arbor, MI (US); Olamide Animasahun, Ann Arbor, MI (US); Tomoharu Yoshizumi, Fukuoka-shi (JP)

(73) Assignees: University of Pittsburgh - Of the Commonwealth System of Higher Education, Pittsburgh, PA (US); Regents of the University of Michigan, Ann Arbor, MI (US); Kyushu University, National University Corporation, Fukuoka-shi (JP)

(21) Appl. No.: 18/712,223

(22) PCT Filed: Nov. 21, 2022
(86) PCT No.: PCT/US2022/080274

§ 371 Date: May 21, 2024

Related U.S. Application Data
(60) Provisional application No. 63/282,051, filed on Nov. 22, 2021.

(51) Int. Cl. A61K 48/00; A61K 31/122; A61K 31/164; A61K 31/355; A61K 31/7105; A61K 33/04; A61K 38/06; A61K 38/44; A61P 1/16; C12N 15/86

(52) U.S. Cl. CPC A61K 48/005; A61K 31/122; A61K 31/164; A61K 31/355; A61K 31/7105; A61K 33/04; A61K 38/063; A61K 38/44; A61P 1/16; C12N 15/86; C12Y 111/01009; C12N 2740/15043; C12N 2750/14143

(57) ABSTRACT

Methods are disclosed herein for promoting survival of a donor liver in a recipient subject, wherein the donor liver is homozygous for rs738409:G mutation in the patatin-like phospholipase domain-containing protein 3 (PNPLA3) gene. The method can include administering to the subject glutathione (GSH) and/or a nucleic acid molecule encoding a component of the ferroptosis pathway (such as GPX4).

### CROSS REFERENCE TO RELATED APPLICATIONS

This claims the benefit of U.S. Provisional Application No. 63/282,051, filed Nov. 21, 2021, which is incorporated by reference herein.

### ACKNOWLEDGMENT OF GOVERNMENT SUPPORT

This invention was made with government support under grant number DK099257 awarded by the National Institutes of Health. The government has certain rights in the invention.

### FIELD

This relates to the field of liver transplants, specifically to methods for increasing survival of liver transplants homozygous for the phospholipase domain-containing protein 3 (PNPLA3) RS738409:G gene.

### BACKGROUND

Approximately 12,000 patients are currently on wait lists for a liver transplantation in the United States, four times more than the number listed in 1998. It is predicted that end-stage liver disease, especially that caused by non-alcoholic fatty liver disease (NAFLD), will increase in the next decade. Therefore, the need for liver transplantation is projected to increase and is already outstripping the supply of deceased donor grafts. To increase the pool for donor grafts many transplant centers have turned their attention to living donor liver transplantation (LDLT) leading to an annual increase of 31%. In some countries, such as Japan, hesitation against deceased organ donation and the definition of brain death emphasize living donor liver transplantation.

Benefits for the recipient of living donor liver transplantation are the ability to perform the transplantation when it is medically indicated before decompensation or death occurs. The donors and the donor grafts can be investigated thoroughly with regards to anatomy, blood type, functionality, mass, liver histology/steatosis etc. The occurrence of primary graft non-function in LDLT is rare due to better donor quality and shorter cold ischemic times.

The regenerative capacity of the human liver allows for the resection of the donor graft and their regeneration into normal size livers in the donor and the recipient several months after transplantation. The undisputable disadvantage is the risk of serious complications or death in the healthy donor undergoing a partial liver resection. Although the overall donor mortality is less than one percent, the most common complications were biliary complications occurring in around six percent of the donors.

Genome-wide association studies have found highly prevalent genetic variants that are associated with liver disease. Therefore, living donor liver transplantation offer the unique opportunity to evaluate potential donor grafts for genetic variants that could influence post-transplantation outcome for the recipient, and provide appropriate treatment for that donor liver. A need remains for methods that increase survival of donor livers with specific genotypes following transplant.

### SUMMARY

Methods are disclosed for promoting survival of a donor liver in a recipient subject, wherein the donor liver is homozygous for rs738409:G mutation in the PNPLA3 gene. These methods include administering to the subject a therapeutically effective amount of one or more of:
(a) a nucleic acid molecule encoding a component of the ferroptosis pathway, wherein the component is glutathione peroxidase 4 (GPX4), glutathione peroxidase 1 (GPX1), glutathione peroxidase 2 (GPX2), glutathione peroxidase 3 (GPX3), ferroptosis suppressor protein 1 (FSP1), stearoyl CoA desaturase (SCD), fatty acid desaturase 2 (FADS2), glutaminase 2 (GLS2), nuclear factor erythroid 2 like 2 (NFE2L2), BTB domain and CNC homolog 1 (BACH1), camp responsive element binding protein (CREB1), G-rich RNA sequence binding factor (GRSF), transcription factor AP-2 gamma (TFAP2C), 3-hydroxy-3-methylglutaryl-CoA reductase (HMGCR), glucokinase (GCK), or cytochrome P450 family 1 subfamily A member 1 (CYP1A1);
(b) a nucleic acid inhibitor of a component of the ferroptosis pathway, wherein the component is acyl-CoA synthetase long chain family member 4 (ACSL4), nuclear receptor coactivator 4 (NCOA4), autophagy related 5 (ATG5), lysophosphatidylcholine acyltransferase 3 (LPCAT3), zinc finger E-box binding homeobox 1 (ZEB1), ST3 beta-galactoside alpha-2,3-sialyltransferase 5 (ST3GAL5), solute carrier family 33 member 1 (SLC33A1), ST3 beta-galactoside alpha-2,3-sialyltransferase 2 (ST3GAL2), ST6 N-acetylgalactosaminide alpha-2,6-sialyltransferase 4 (ST6GALNAC4), heparan sulfate-glucosamine 3-sulfotransferase 3A1 (HS3ST3A1), beta-1,4-N-acetyl-galactosaminyltransferase 1 (B4GALNT1), ST6 N-acetylgalactosaminide alpha-2,6-sialyltransferase 6 (ST6GALNAC6), hexosaminidase subunit beta (HEXB), hyaluronidase 4 (HYAL4), or glucosamine (N-Acetyl)-6-sulfatase (GNS);
(c) glutathione (GSH) or a nucleic acid molecule encoding an enzyme that increases glutathione production, wherein the enzyme is glutaminase 2 (GLS2), solute carrier family 3 member 2 (SLC3A2), solute carrier family 7 member 11 (SLC7A11), solute carrier family 38 member 1 (SLC38A1), solute carrier family 1 member 5 (SLC1A5), or glutathione reductase (GSR); or
(d) a supplement that increases glutathione in the subject, wherein the supplement is at least one of Glutamine, Glutamate, glutathione disulfide (GSSG), Cysteine, Glycine, Cys-Gly, Choline, Phosphatidylcholine, Sorbitol, Palmitoylcarnitine, Methylthioadenosine, S-Adenosylhomocysteine, S-Adenosylmethionine, Cystine, Methionine Creatine, Gamma-aminobutyrate, or Guanadinoacetate.

In some embodiments herein are methods of promoting survival of a donor liver in a recipient subject, wherein the donor liver is homozygous for rs738409:G mutation in the PNPLA3 gene. The methods include administering to the subject (a) a nucleic acid molecule encoding GPX4 or (b) GSH. In some embodiments, the methods further include detecting the rs738409:G mutation in a sample from the donor. Yet other embodiments include administering to the donor liver a nucleic acid molecule encoding GPX4, such as in an ex vivo perfusion system. The subject may be a recipient of a cadaveric liver transplant or a recipient of a liver transplant from a living donor.

The foregoing and other objects, features, and advantages of the invention will become more apparent from the following detailed description, which proceeds with reference to the accompanying figures.

### BRIEF DESCRIPTION OF THE DRAWINGS

FIG. 1 shows a schematic of the living donor liver transplantation, the investigated variants, and the in vitro analysis process described herein.

FIGS. 2A-2F describe genetic variants in the population study. Six single nucleotide polymorphisms (SNPs) were found to be correlated with fatty liver disease and nonalcoholic steatohepatitis (NASH) in previous genome wide association studies (GWAS). The coding strands with the major alleles of the six SNPs (PNPLA3 rs738409, MBOAT7 rs641738, TM6SF2 rs58542926, HSD17B13 rs72613567, GCKR rs780094, and TCF7L2 rs7903146) are shown with the minor allele beneath (FIG. 2A). SNPs are highly prevalent among healthy individuals (donors) and patients with end stage liver disease (recipients) undergoing living donor liver transplantation. Homozygosity for PNPLA3 rs738409:G is prevalent in around 50% of the recipients and 30% of the donors, and GCKR rs780094:T homozygosity is prevalent in around 25% of donors and recipients (FIG. 2B). Recipients who were homozygous for the PNPLA3 rs738409:G or the GCKR rs780094:T variant had a 15% chance of receiving a donor organ that was also homozygous for the same SNPs (FIG. 2C). Combinations of different polymorphisms in the donors and recipients were highly prevalent, with more than 50% of donors and 70% of recipients having a minor homozygosity for one of the SNPs (FIG. 2D). Venn diagram of minor homozygosities of donors and recipients of living donor liver transplantation. More common SNPs homozygosities were commonly carried by the same individuals (e.g., PNPLA3 or GCKR) (FIGS. 2E-2F).

FIGS. 3A-3B show a donor (FIG. 3A) and recipient (FIG. 3B) survival analysis based on genotype. Kaplan-Meier estimate was used to compare a recipient's 5-year survival with the genotyping results of the recipient and the donor graft (Log-rank Mantel-Cox test). Recipients with a donor graft homozygous for the rs738409:G variant showed significantly reduced survival (p=0.03, Log-rank Mantel-Cox test).

FIGS. 4A-4E show a dual graft recipient case. One patient received two donor grafts negative for hepatic steatosis before transplantation (<0.5% hepatic steatosis) (FIG. 4A). The right lobe graft was heterozygous for the PNPLA3 rs738409:G variant and the GCKR rs780094:T variant. The left lobe graft was homozygous for the PNPLA3 rs738409:G variant and the GCKR rs780094:T variant. The left lobe graft developed postoperative steatosis whereas the right lobe graft did not develop postoperative steatosis, indicating a potential underlying genetic difference leading to hepatic steatosis (FIG. 4B). Before transplantation, donor 2 began a diet, reducing hepatic steatosis from 20% to <5% (FIG. 4C). Donors and recipients in the dual graft case were genotyped for PNPLA3 rs738409, MBOAT7 rs641738, TM6SF2 rs58542926, HSD17B13 rs72613567, GCKR rs780094 and TCF7L2 rs7903146. Only minor homozygosities for GCKR (recipient, donor2) and PNPLA3 (donor2) were found in a genotype cluster plot analysis (FIGS. 4D and 4E).

FIGS. 5A-5D show an omics analysis of primary human hepatocytes. Metabolomic analysis of primary human hepatocytes homozygous for the PNPLA3 rs738409:G variant revealed an upregulation in the ferroptosis pathway (here referred to as glutathione metabolism) (FIGS. 5A-5B). RNAseq analysis showed alterations in genes related to lipid storage in hepatocytes positive for the PNPLA3 rs738409:G variant and also for the uptake of fatty acids, leading to differences in Glycero(phospho)lipid metabolism and fatty acid degradation (FIGS. 5C-5D).

FIG. 6 shows quantification of ferroptosis metabolites in primary human hepatocytes. Hepatocytes positive for the PNPLA3 rs738409:G variant have upregulated ferroptosis metabolites (glutamate, glutathione, sorbitol, creatine, phosphorylcholine, choline), representing an ongoing ferroptosis process.

FIGS. 7A-7I show that causal ferroptosis therapy can improve cell survival in donor hepatocytes exhibiting signs of ferroptosis. PNPLA3 rs738409:G homozygous donor hepatocytes showed high amounts of lipid droplets and signs of ferroptosis, including lipid peroxidation, reduced expression levels of GPX4 and mitochondrial shrinkage (FIG. 7A). Alterations in the metabolism of fatty acids increase susceptibility to lipid peroxidation (e.g., polyunsaturated fatty acids (PUFAs) and increase ferroptosis (FIGS. 7B-7C). Compensatory iron storage reveals cellular protection mechanisms (FIGS. 7D-7E). Upon iron oxidation, the protection mechanisms help to reduce ferroptosis susceptibility, but donor hepatocytes remain sensible to ferroptosis (FIG. 7FD). Treatment with an approved iron chelator (deferoxamine) reduces lipid peroxidation, improves mitochondrial shape, and increases cell survival (FIG. 7G). L-Glutathione (GSH) supplementation reduces lipid peroxidation and improves cell survival (FIG. 7H). Gene therapy inducing GPX4 expression restores GPX4 levels and improves cell survival (FIG. 7I).

FIG. 8A-8B. show a schematic of the ferroptosis pathway (FIG. 8A) and the main proteins modulating iron metabolism in ferroptosis (FIG. 8B). Figure is from Chen et al., Frontiers in Cell and Developmental Biology, 8:590226, 2020.

### SEQUENCE LISTING

The nucleic acid sequences listed in the accompanying sequence listing are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an Extensible Markup Language (XML) file in the form of the file named 8123-106924-02 ST26 SL.xml, which was created on Nov. 10, 2022and is 7, 751 bytes, which is incorporated by reference herein.
SEQ ID NO: 1 is a PNPLA3 (forward) sequencing primer.
SEQ ID NO: 2 is a PNPLA3 (reverse) sequencing primer.
SEQ ID NO: 3 is the nucleotide sequence of human GPX4 transcript variant NM_001039848.4.
SEQ ID NO: 4 is the nucleotide sequence of human GPX4 transcript variant NM_001039848.4 modified to lack a poly-A-sequence and to include a selenocysteine insertion sequence (SECIS) element.

### DETAILED DESCRIPTION

Provided herein are methods for promoting survival of a donor liver in a recipient subject, wherein the donor liver is homozygous for rs738409:G mutation in the PNPLA3 gene. Genome-wide association studies (GWAS) have uncovered an important variant in the PNPLA3 gene (rs738409:G). The PNPLA3 gene encodes for the patatin-like phospholipase domain-containing that is located on the lipid droplets of hepatocytes and has a lipase activity. The PNPLA3 rs738409:G variant is found in 30%-50% of individuals worldwide and it is associated with hepatic steatosis, nonalcoholic steatohepatitis (NASH), alcoholic and nonalcoholic cirrhosis and even hepatocellular carcinoma. The PNPLA3 rs738409:G variant leads to a loss of function promoting the accumulation of triglycerides in the lipid droplet of the hepatocyte. Hepatic triglycerides are especially enriched in PUFAs in carriers with the PNPLA3 rs738409:G variant. Disclosed herein are treatments that improve hepatocyte survival in donor livers from subject that have the PNPLA3 rs738409:G variant.

The Examples provided herein show that the presence of the PNPLA3 rs738409:G variant in a donor liver is associated with reduced recipient survival and is associated with an increase in donor graft lipid peroxidation. PNPLA3 rs738409:G variant-carrying donor livers exhibited metabolic changes, mitochondrial shrinkage, and reduced GPX4 expression associated with a form of iron dependent cell death (“ferroptosis”) in donor hepatocytes. In some embodiments, restoring GPX4 expression using gene therapy treated ferroptosis in hepatocytes carrying the PNPLA3 variant. Further, iron chelation and nutritional supplements successfully treated ferroptosis in donor grafts carrying the PNPLA3 variant. As disclosed herein, GPX4 and other molecules relevant to PNPLA3-induced ferroptosis can be targeted in donor liver grafts in order to increase hepatocyte, and thus recipient, survival.

In some embodiments, the nucleic acid molecule encoding GPX4 is an mRNA that can be administered to a subject, for example, using lipid nanoparticles (LNP), using polymeric nanoparticles, as a conjugate to N-acetylgalactosamine (GalNAc), as an mRNA modified by base linker sugars, using a degradable polymer, as an mRNA-Lipoplex, or as mRNA cargo of PEG-10. In other embodiments, the subject is administered a viral vector, such as a lentiviral or adenoviral vector, comprising the nucleic acid molecule encoding GPX4.

In certain embodiments, the subject is further administered one or more additional therapeutics, such as deferoxamine (DFO), selenium, vitamin E (alpha-tocopherol), CoQ10, or a combination thereof. Also disclosed herein are compositions comprising (a) a nucleic acid molecule encoding GPX4 or (b) GSH, for use in the disclosed methods.

### I. Terms

Unless otherwise noted, technical terms are used according to conventional usage. Definitions of common terms in molecular biology can be found in Krebs et al (Eds.), Lewin's Genes XII, published by Jones & Bartlett Publishers, 2017; and Meyers et al. (Eds.), The Encyclopedia of Cell Biology and Molecular Medicine, published by Wiley-VCH in 16 volumes, 2008; and other similar references.

As used herein, the singular forms “a,” “an,” and “the” refer to both the singular as well as plural unless the context clearly indicates otherwise. Further, “or” also include “and/or”; thus, “a lentivirus vector or a adenovirus vector” also includes “a lentivirus vector and/or an adenovirus vector,” and compositions of use in the methods herein can be used alone or in combination. Although many methods and materials similar or equivalent to those described herein can be used, particular suitable methods and materials are described below. The term “comprises” means “includes.” The term “about” means within five percent, unless otherwise indicated. All publications, patent applications, patents, and other references mentioned herein are incorporated by reference in their entirety. In case of conflict, the present specification, including explanations of terms, will control. In addition, the materials, methods, and examples are illustrative only and not intended to be limiting. To facilitate review of the various embodiments, the following explanations of terms are provided:

3-hydroxy-3-methylglutaryl-CoA reductase (HMGCR): The rate-limiting enzyme for cholesterol synthesis and is regulated via a negative feedback mechanism mediated by sterols and non-sterol metabolites derived from mevalonate, the product of the reaction catalyzed by reductase. HMGCR catalyzes the conversion of (3S)-hydroxy-3-methylglutaryl-CoA (HMG-CoA) to mevalonic acid, the rate-limiting step in the synthesis of cholesterol and other isoprenoids, and thus plays a role in cellular cholesterol homeostasis. In mammalian cells, this enzyme is typically suppressed by cholesterol derived from the internalization and degradation of low density lipoprotein (LDL) via the LDL receptor. Competitive inhibitors of HMGCR induce expression of LDL receptors in the liver, which in turn increases catabolism of plasma LDL and lowers the plasma concentration of cholesterol, a determinant of atherosclerosis. Exemplary HMGCR nucleic acid sequences are provided in GENBANK® Accession Nos. NM_000859, NM_001130996, and NM_001364187, as available on Nov. 12, 2021. Exemplary HMGCR amino acid sequences are provided in GENBANK® Accession Nos. NP_000850, NP_001124468, and NP_001351116, as available on Nov. 12, 2021.

Acyl-CoA synthetase long chain family member 4 (ACSL4): Isozyme 4 of the long-chain fatty-acid-coenzyme A ligase family. This isozyme converts free long-chain fatty acids into fatty acyl-CoA esters, and thereby play roles in lipid biosynthesis and fatty acid degradation. ACSL4 preferentially utilizes arachidonate as substrate. Exemplary ACSL4 nucleic acid sequences are provided in GENBANK® Accession Nos. NM_001318509, NM_001318510, NM_004458, and NM_022977, as available on Nov. 12, 2021. Exemplary ACSL4 amino acid sequences are provided in GENBANK® Accession Nos. NP_001305438, NP_001305439, NP_004449, and NP_075266, as available on Nov. 12, 2021. Administration: To provide or give a subject an agent, such as a therapeutic agent (e.g. GSH or a nucleic acid encoding GPX4), by any effective route. Exemplary routes of administration are described herein.

Autophagy related 5 (ATG5): A protein which, in combination with autophagy protein 12 (ATG12), functions as an E1-like activating enzyme in a ubiquitin-like conjugating system. ATG5 is involved in several cellular processes, including autophagic vesicle formation, mitochondrial quality control after oxidative damage, negative regulation of the innate antiviral immune response, lymphocyte development and proliferation, MHC II antigen presentation, adipocyte differentiation, and apoptosis. Exemplary ATG5 nucleic acid sequences are provided in GENBANK® Accession Nos. NM_001286106, NM_001286107, NM_001286108, NM_001286111, and NM_004849, as available on Nov. 12, 2021. Exemplary ATG5 amino acid sequences are provided in GENBANK® Accession Nos. NP_001273035, NP_001273036, NP_001273037, NP_001273040, and NP_004840, as available on Nov. 12, 2021.

Beta-1,4-N-acetyl-galactosaminyltransferase 1 (B4GALNT1): An enzyme involved in the biosynthesis of G(M2) and G(D2) glycosphingolipids. GM2 and GD2 gangliosides are sialic acid-containing glycosphingolipids. GalNAc-T catalyzes the transfer of GalNAc into G(M3) and G(D3) by a beta-1,4 linkage, resulting in the synthesis of G(M2) and G(D2), respectively. B4GALNT1 is also involved in the biosynthesis of gangliosides GT2 and GA2 from GT3 and GA3, respectively. Exemplary B4GALNT1 nucleic acid sequences are provided in GENBANK® Accession Nos. NM_001276468, NM_001276469, and NM_001478, as available on Nov. 12, 2021. Exemplary B4GALNT1 amino acid sequences are provided in GENBANK® Accession Nos. NP_001263397, NP_001263398, and NP_001469, as available on Nov. 12, 2021.

BTB domain and CNC homolog 1 (BACH1): A transcription factor that belongs to the cap‘n’collar type of basic region leucine zipper factor family (CNC-bZip). The protein contains broad complex, tramtrack, bric-a-brac/poxvirus and zinc finger (BTB/POZ) domains, which is atypical of CNC-bZip family members. These BTB/POZ domains facilitate protein-protein interactions and formation of homo- and/or hetero-oligomers. When this encoded protein forms a heterodimer with MafK, it functions as a repressor of Maf recognition element (MARE) and transcription is repressed. Thus, together with MAF, NFE2L2 represses the transcription of genes under the control of the NFE2L2 oxidative stress pathway. Exemplary BACH1 nucleic acid sequences are provided in GENBANK® Accession Nos. NM_001186 and NM_206866, as available on Nov. 12, 2021. Exemplary BACH1 amino acid sequences are provided in GENBANK® Accession Nos. NP_001177 and NP_996749, as available on Nov. 12, 2021.

Camp responsive element binding protein (CREB1): A transcription factor that is a member of the leucine zipper family of DNA binding proteins. This protein binds as a homodimer to the cAMP-responsive element, an octameric palindrome. CREB1 is phosphorylated by several protein kinases, and induces transcription of genes in response to hormonal stimulation of the cAMP pathway. Exemplary CREB1 nucleic acid sequences are provided in GENBANK® Accession Nos. NM_001320793, NM_001371426, NM_001371427, NM_001371428, NM_004379, and NM_134442, as available on Nov. 12, 2021. Exemplary CREB1 amino acid sequences are provided in GENBANK® Accession Nos. NP_001307722, NP_001358355, NP_001358356, NP_001358357, NP_004370, and NP_604391, as available on Nov. 12, 2021.

Cytochrome P450 family 1 subfamily A member 1 (CYP1A1): A member of the cytochrome P450 superfamily of enzymes. The cytochrome P450 proteins are monooxygenases which catalyze many reactions involved in drug metabolism and synthesis of cholesterol, steroids and other lipids. CYP1A1 is involved in the metabolism of various endogenous substrates, including fatty acids, steroid hormones and vitamins. This protein localizes to the endoplasmic reticulum and its expression is induced by some polycyclic aromatic hydrocarbons (PAHs), some of which are found in cigarette smoke. The enzyme's endogenous substrate is unknown; however, it is able to metabolize some PAHs to carcinogenic intermediates. Exemplary CYP1A1 nucleic acid sequences are provided in GENBANK® Accession Nos. NM_000499 and NM_001319216, as available on Nov. 12, 2021. Exemplary CYP1A1 amino acid sequences are provided in GENBANK® Accession Nos. NP_000490 and NP_001306145, as available on Nov. 12, 2021.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, nucleic acid sequences whose presence can influence expression, and can also include additional nucleic acid sequences whose presence is advantageous, for example, leader sequences and fusion partner sequences. Expression control sequences can include a promoter.

Fatty acid desaturase 2 (FADS2): A member of the fatty acid desaturase (FADS) gene family. Desaturase enzymes regulate unsaturation of fatty acids through the introduction of double bonds between defined carbons of the fatty acyl chain. FADS family members are fusion products composed of an N-terminal cytochrome b5-like domain and a C-terminal multiple membrane-spanning desaturase portion, both of which are characterized by conserved histidine motifs. FADS2 is involved in the biosynthesis of highly unsaturated fatty acids (HUFA) from the essential PUFAs linoleic acid (LA) (18:2n-6) and alpha-linolenic acid (ALA) (18:3n-3) precursors, acting as a fatty acyl-coenzyme A desaturase that introduces a cis double bond at carbon 6 of the fatty acyl chain. FADS2 catalyzes the first and rate limiting step in this pathway, which is the desaturation of LA (18:2n-6) and ALA (18:3n-3) into gamma-linoleate (GLA) (18:3n-6) and stearidonate (18:4n-3), respectively. Exemplary FADS2 nucleic acid sequences are provided in GENBANK® Accession Nos. NM_001281501, NM_001281502, and NM_004265, as available on Nov. 12, 2021. Exemplary FADS2 amino acid sequences are provided in GENBANK® Accession Nos. NP_001268431, NP_001268430, and NP_004256, as available on Nov. 12, 2021.

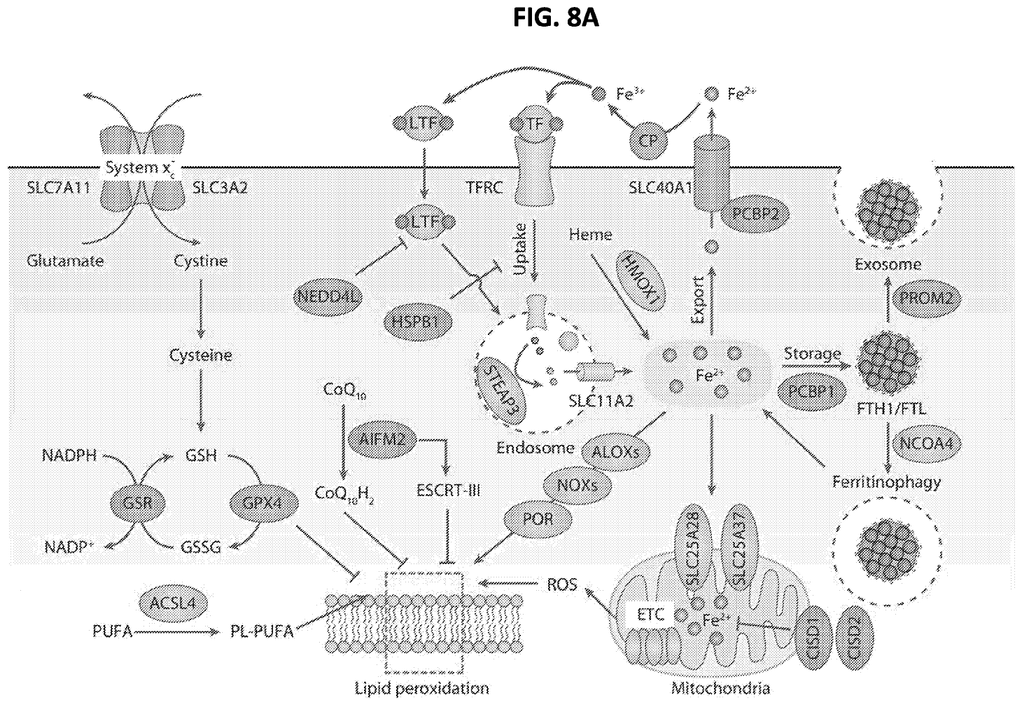

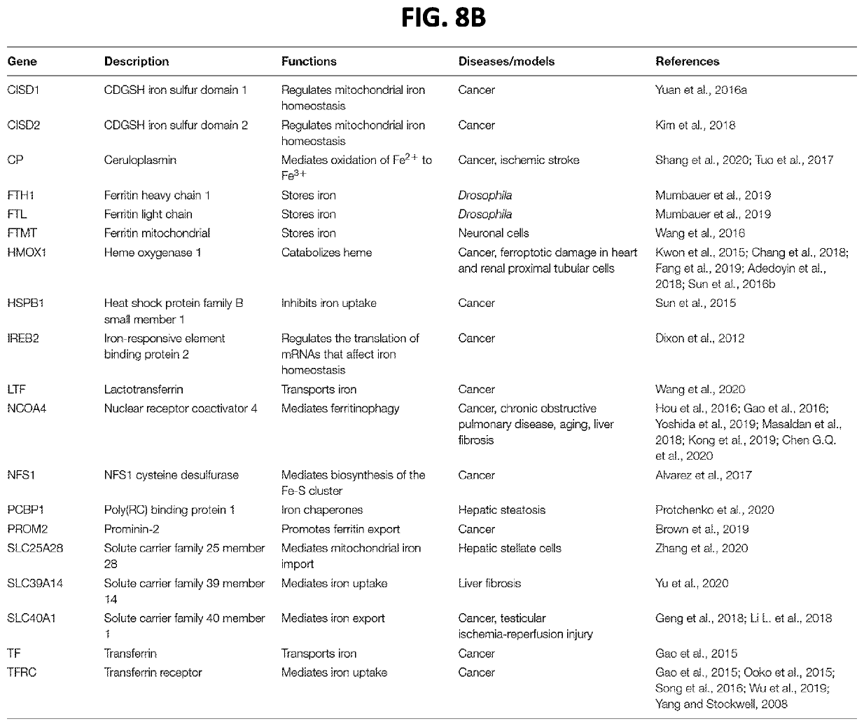

Ferroptosis pathway: Ferroptosis is a form of oxidative cell death that is characterized by iron-dependent oxidative damage and subsequent plasma membrane rupture and the release of damage-associated molecular patterns. It is induced by the accumulation of iron-mediated lipid peroxidation. Ferroptotic cells show typical necrotic morphology, such as an incomplete plasma membrane and the release of intracellular contents, especially damage-associated molecular patterns (DAMPs). Due to the role of iron in mediating the production of reactive oxygen species and enzyme activity in lipid peroxidation, ferroptosis is controlled by regulators involved in many aspects of iron metabolism, such as iron uptake, storage, utilization, and efflux. The ferroptosis pathway is shown in FIG. 8A. The main proteins modulating iron metabolism in ferroptosis are shown in FIG. 8B. Ferroptosis can be caused by the collapse of the glutathione (GSH)-glutathione peroxidase 4 (GPX4) antioxidant systems. System xc- is a heterodimeric transmembrane complex composed of light chain, solute carrier family 7 member 11 (SLC7A11/xCT), and heavy chain, solute carrier family 3 member 2 (SLC3A2). After entering the cells by system xc-, cystine is quickly reduced to cysteine, which is mainly utilized for the synthesis of GSH. As a potent, low molecular weight antioxidant in cells, GSH is utilized by GPX4, which uses highly nucleophilic selenocysteine to reduce lipid peroxides into lipid alcohols. The pharmacological inhibitors of system xc- (e.g., erastin) and GPX4 (e.g., RSL3) are the two classical ferroptosis inducers. In addition, several GPX4-independent anti-ferroptosis pathways have recently been identified, such as the apoptosis-inducing factor mitochondria-associated 2 (AIFM2)-mediated CoQ10 production pathway and the endosomal sorting complex required for transport-III (ESCRT-III)-dependent membrane repair pathway. Lipid peroxidation results in the oxidation of polyunsaturated fatty acids (PUFAs) of membrane lipids. An impaired antioxidant system can cause or accelerate lethal lipid peroxidation, which is inhibited by various synthetic antioxidants (e.g., ferrostatin-1 and liproxstatin-1). Acyl-CoA synthetase long-chain family member 4 (ACSL4) is a crucial pro-ferroptotic regulator that catalyzes the synthesis of long-chain polyunsaturated CoAs, especially arachidonic acid, thus enriching cellular membranes with PUFA. By way of further examples, nicotinamide adenine dinucleotide phosphate (NADPH) oxidases (NOXs), lipoxygenases (ALOXs), and cytochrome P450 oxidoreductase (POR) coupled to cytochrome P450 (CYP) monooxygenases, are all involved in the ferroptosis pathway. A “component” of the ferroptosis pathway is any enzyme or other protein that plays a role in ferroptosis.

Ferroptosis suppressor protein 1 (FSP1): An NAD(P)H-dependent oxidoreductase involved in cellular oxidative stress response. At the plasma membrane, FSP1 catalyzes reduction of coenzyme Q/ubiquinone-10 to ubiquinol-10, a lipophilic radical-trapping antioxidant that prevents lipid oxidative damage and consequently ferroptosis. It cooperates with GPX4 to suppress phospholipid peroxidation and ferroptosis independently of cellular glutathione levels. FSP1 may play a role in mitochondrial stress signaling. Upon oxidative stress, it associates with the lipid peroxidation end product 4-hydroxy-2-nonenal (HNE), forming a lipid adduct devoid of oxidoreductase activity, which then translocates from mitochondria into the nucleus triggering DNA damage and cell death. FSP1 is capable of DNA binding in a non-sequence specific way. Exemplary FSP1 nucleic acid sequences are provided in GENBANK® Accession Nos. NM_002961 and NM_019554, as available on Nov. 12, 2021. Exemplary FSP1 amino acid sequences are provided in GENBANK® Accession Nos. NP_002952 and NP_06242, as available on Nov. 12, 2021.

Glucokinase (GCK): A member of the hexokinase family of proteins. Hexokinases phosphorylate glucose to produce glucose-6-phosphate, the first step in most glucose metabolism pathways. GCK catalyzes the phosphorylation of hexose, such as D-glucose, D-fructose, and D-mannose, to hexose 6-phosphate (D-glucose 6-phosphate, D-fructose 6-phosphate, and D-mannose 6-phosphate, respectively). In contrast to other forms of hexokinase, GCK is not inhibited by its product glucose-6-phosphate but remains active while glucose is abundant. The use of multiple promoters and alternative splicing of this gene result in distinct protein isoforms that exhibit tissue-specific expression in the pancreas and liver. In the pancreas, this enzyme plays a role in glucose-stimulated insulin secretion, while in the liver, this enzyme is important in glucose uptake and conversion to glycogen. Mutations in this gene that alter enzyme activity have been associated with multiple types of diabetes and hyperinsulinemic hypoglycemia. Exemplary GCK nucleic acid sequences are provided in GENBANK® Accession Nos. NM_000162, NM_001354800, NM_001354801, NM_001354802, NM_001354803, NM_033507, and NM_033508, as available on Nov. 12, 2021. Exemplary GCK amino acid sequences are provided in GENBANK® Accession Nos. NP_000153, NP_001341729, NP_001341730, NP_001341731, NP_001341732, NP_277042, and NP_277043, as available on Nov. 12, 2021.

Glucosamine (N-Acetyl)-6-sulfatase (GNS): A lysosomal enzyme found in all cells. It is involved in the catabolism of heparin, heparan sulphate, and keratan sulphate. The human liver contains two major forms of GNS: form A, which has a single 78 kDa polypeptide, and form B, which has two polypeptides of 48 kDa and 32 kDa. Exemplary GNS nucleic acid and amino acid sequences are provided in GENBANK® Accession No. NM_002076 and NP_002067, respectively, as available on Nov. 12, 2021.

Glutaminase 2 (GLS2): A mitochondrial phosphate-activated glutaminase that catalyzes the hydrolysis of glutamine to glutamate and ammonia. Originally thought to be liver-specific, this protein has been found in other tissues as well. GLS2 plays a role in the regulation of glutamine catabolism and promotes mitochondrial respiration and increases ATP generation in cells by catalyzing the synthesis of glutamate and alpha-ketoglutarate. It increases cellular anti-oxidant function via NADH and glutathione production, and may play a role in preventing tumor proliferation. Exemplary GLS2 nucleic acid sequences are provided in GENBANK® Accession Nos. NM_001280796, NM_001280797, NM_001280798, and NM_013267, as available on Nov. 12, 2021. Exemplary GLS2 amino acid sequences are provided in GENBANK® Accession Nos. NP_001267725, NP_001267726, NP_001267727, and NP_037399, as available on Nov. 12, 2021.

Glutathione peroxidase 1 (GPX1): A protein belonging to the glutathione peroxidase family, members of which catalyze the reduction of organic hydroperoxides and hydrogen peroxide (H₂O₂) by glutathione, and thereby protect cells against oxidative damage. H₂O₂ is also important in growth-factor mediated signal transduction, mitochondrial function, and maintenance of thiol redox-balance; therefore, by limiting H₂O₂ accumulation, glutathione peroxidases are also involved in modulating these processes. Several isozymes of this gene family exist in vertebrates, which vary in cellular location and substrate specificity. This isozyme is the most abundant, is ubiquitously expressed and localized in the cytoplasm, and whose preferred substrate is hydrogen peroxide. It is also a selenoprotein, containing the rare amino acid selenocysteine (Sec) at its active site. Sec is encoded by the UGA codon, which normally signals translation termination. The 3′ UTRs of selenoprotein mRNAs contain a conserved stem-loop structure, designated the Sec insertion sequence (SECIS) element, that is necessary for the recognition of UGA as a Sec codon, rather than as a stop signal. Exemplary GPX1 nucleic acid sequences are provided in GENBANK® Accession Nos. NM_000581, NM_001329455, NM_001329502, NM_001329503, and NM_201397, as available on Nov. 12, 2021. Exemplary GPX1 amino acid sequences are provided in GENBANK® Accession Nos. NP_000572, NP_001316384, NP_001316431, NP_001316432, and NP_958799, as available on Nov. 12, 2021.

Glutathione peroxidase 2 (GPX2): A protein belonging to the glutathione peroxidase family, members of which catalyze the reduction of organic hydroperoxides and hydrogen peroxide (H₂O₂) by glutathione, and thereby protect cells against oxidative damage. Several isozymes of this gene family exist in vertebrates, which vary in cellular location and substrate specificity. This isozyme is predominantly expressed in the gastrointestinal tract (also in liver in human) and is localized in the cytoplasm. The GPX2 preferred substrate is hydrogen peroxide. Overexpression of GPX2 is associated with increased differentiation and proliferation in colorectal cancer. Like GPX1 and GPX3, GPX2 is also a selenoprotein, containing the rare amino acid selenocysteine (Sec) at its active site. Exemplary GPX2 nucleic acid and amino acid sequences are provided in GENBANK® Accession Nos. NM_002083 and NP_002074, respectively, as available on Nov. 12, 2021.

Glutathione peroxidase 3 (GPX3): A protein belonging to the glutathione peroxidase family, members of which catalyze the reduction of organic hydroperoxides and hydrogen peroxide (H₂O₂) by glutathione, and thereby protect cells against oxidative damage. Several isozymes of this gene family exist in vertebrates, which vary in cellular location and substrate specificity. This isozyme is secreted and is abundantly found in plasma. Downregulation of GPX3 expression by promoter hypermethylation has been observed in a wide spectrum of human malignancies, including thyroid cancer, hepatocellular carcinoma and chronic myeloid leukemia. Like GPX1 and GPX2, GPX3 is also a selenoprotein, containing the rare amino acid selenocysteine (Sec) at its active site. Exemplary GPX3 nucleic acid sequences are provided in GENBANK® Accession Nos. NM_001329790 and NM_002084, as available on Nov. 12, 2021. Exemplary GPX3 amino acid sequences are provided in GENBANK® Accession Nos. NP_001316719 and NP_002075, as available on Nov. 12, 2021.

Glutathione peroxidase 4 (GPX4): A phospholipid hydroperoxidase that protects cells against membrane lipid peroxidation. GPX4 belongs to the family of glutathione peroxidases, which consists of eight known mammalian isoenzymes (GPX1-8). GPX4 catalyzes the reduction of hydrogen peroxide, organic hydroperoxides, and lipid peroxides at the expense of reduced glutathione, and functions in the protection of cells against oxidative stress. The oxidized form of glutathione (glutathione disulfide), which is generated during the reduction of hydroperoxides by GPX4, is recycled by glutathione reductase and NADPH/H+. GPX4 differs from the other GPX family members in terms of its monomeric structure, a less restricted dependence on glutathione as reducing substrate, and the ability to reduce lipid-hydroperoxides inside biological membranes. Inactivation of GPX4 leads to an accumulation of lipid peroxides, resulting in ferroptotic cell death. Exemplary GPX4 nucleic acid sequences are provided in GENBANK® Accession Nos. NM_001039847, NM_001039848, NM_001367832, and NM_002085, as available on Nov. 12, 2021. Exemplary GPX4 amino acid sequences are provided in GENBANK® Accession Nos. NP_001034936, NP_001034937, NP_001354761, and NP_002076, as available on Nov. 12, 2021.

Glutathione reductase (GSR): A member of the class-I pyridine nucleotide-disulfide oxidoreductase family. This enzyme is a homodimeric flavoprotein. It is a central enzyme of cellular antioxidant defense and reduces oxidized glutathione disulfide to the sulfhydryl form GSH, which is an important cellular antioxidant. Exemplary GSR nucleic acid sequences are provided in GENBANK® Accession Nos. NM_000637, NM_001195102, NM_001195103, and NM_001195104, as available on Nov. 12, 2021. Exemplary GSR amino acid sequences are provided in GENBANK® Accession Nos. NP_000628, NP_001182031, NP_001182032, and NP_001182033, as available on Nov. 12, 2021.

G-rich RNA sequence binding factor (GRSF1): A protein that binds RNAs containing the G-rich element. The protein is localized in the cytoplasm and has been shown to stimulate translation of viral mRNAs in vitro. GRSF1 is a regulator of post-transcriptional mitochondrial gene expression, required for assembly of the mitochondrial ribosome and for recruitment of mRNA and lncRNA. It binds RNAs containing the 14 base G-rich element. GRSF1 preferentially binds RNAs transcribed from three contiguous genes on the light strand of mtDNA, the mitochondrially encoded NADH:ubiquinone oxidoreductase core subunit 6 (MT-ND6) mRNA, and the long non-coding RNAs for mitochondrially encoded cytochrome B (MT-CYB) and mitochondrially encoded NADH:ubiquinone oxidoreductase core subunit 5 (MT-ND5), each of which contains multiple consensus binding sequences. Exemplary GRSF1 nucleic acid sequences are provided in GENBANK® Accession Nos. NM_001098477 and NM_002092, as available on Nov. 12, 2021. Exemplary GRSF1 amino acid sequences are provided in GENBANK® Accession Nos. NP_001091947 and NP_002083, as available on Nov. 12, 2021.

Heparan sulfate-glucosamine 3-sulfotransferase 3A1 (HS3ST3A1): A member of the heparan sulfate biosynthetic enzyme family. It is a type II integral membrane protein and possesses heparan sulfate glucosaminyl 3-O-sulfotransferase activity. The sulfotransferase domain of this enzyme is highly similar to the same domain of heparan sulfate D-glucosaminyl 3-O-sulfotransferase 3B1, and these two enzymes sulfate an identical disaccharide. This gene is widely expressed, with the most abundant expression in liver and placenta. HS3ST3A1 is a sulfotransferase that utilizes 3′-phospho-5′-adenylyl sulfate (PAPS) to catalyze the transfer of a sulfo group to an N-unsubstituted glucosamine linked to a 2-O-sulfo iduronic acid unit on heparan sulfate. It catalyzes the O-sulfation of glucosamine in IdoUA2S-GlcNS and also in IdoUA2S-GlcNH2. Unlike 3-OST-1, it does not convert non-anticoagulant heparan sulfate to anticoagulant heparan sulfate. Exemplary HS3ST3A1 nucleic acid and amino acid sequences are provided in GENBANK® Accession Nos. NM_006042 and NP_006033, respectively, as available on Nov. 12, 2021.

Hepatocyte: A cell of the main parenchymal tissue of the liver, that make up 70-85% of the mass of the liver. The typical hepatocyte is cubical with sides of 20-30 μm, and produces serum albumin, fibrinogen, and the prothrombin group of clotting factors (except for Factors 3 and 4). Hepatocytes also synthesize lipoproteins, ceruloplasmin, transferrin, complement, and glycoproteins. A hepatocyte is a normal (non-malignant) cell.

Heterologous: A heterologous sequence is a sequence that is not normally (in the wild-type sequence) found adjacent to a second sequence. In one embodiment, the sequence is from a different genetic source, such as a virus or organism, than the second sequence. In another embodiment, the heterologous sequence is a recombinant sequence that is not normally next to the wild-type sequence.

Hexosaminidase subunit beta (HEXB): A subunit of the lysosomal enzyme beta-hexosaminidase that, together with the cofactor GM2 activator protein, catalyzes the degradation of the ganglioside GM2, and other molecules containing terminal N-acetyl hexosamines. Beta-hexosaminidase is composed of two subunits, alpha and beta, which are encoded by separate genes. Both beta-hexosaminidase alpha and beta subunits are members of family 20 of glycosyl hydrolases. HEXB hydrolyzes the non-reducing end N-acetyl-D-hexosamine and/or sulfated N-acetyl-D-hexosamine of glycoconjugates, such as the oligosaccharide moieties from proteins and neutral glycolipids, or from certain mucopolysaccharides. The isozyme B does not hydrolyze each of these substrates; however, it hydrolyzes efficiently neutral oligosaccharide. Only the isozyme A is responsible for the degradation of GM2 gangliosides in the presence of GM2A. Exemplary HEXB nucleic acid sequences are provided in GENBANK® Accession Nos. NM_000521 and NM_001292004, as available on Nov. 12, 2021. Exemplary HEXB amino acid sequences are provided in GENBANK® Accession Nos. NP_000512 and NP_001278933, as available on Nov. 12, 2021.

Hyaluronidase 4 (HYAL4): A protein that is similar in structure to hyaluronidases but lacks hyaluronidase activity. The encoded protein acts as a chondroitin-sulfate-specific endo-beta-N-acetylgalactosaminidase, as it exhibits hydrolytic activity toward chondroitin sulfate chains and degrades them into oligosaccharides. Proteoglycans are formed by the covalent linkage of chondroitin sulfate chains to protein. Proteoglycans are part of the extracellular matrix of connective tissues and are also found at the surface of many cell types where they participate in a variety of cellular processes such as cell proliferation, differentiation, migration, cell-cell recognition, extracellular matrix deposition, and tissue morphogenesis. Exemplary HYAL4 nucleic acid sequences are provided in GENBANK® Accession Nos. XM_017011911, XM_011515990, and XM_024446703, as available on Nov. 12, 2021. Exemplary HYAL4 amino acid sequences are provided in GENBANK® Accession Nos. XP_016867400, XP_011514292 and XP_024302471, as available on Nov. 12, 2021.

Inhibiting or treating a disease: Inhibiting the full development of a disease or condition, for example, in a subject who is at risk for a disease such as transplant rejection. “Treatment” refers to a therapeutic intervention that ameliorates a sign or symptom of a disease or pathological condition, such as transplant rejection, after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease, such as improved survival of a transplant. Treatment may be assessed by objective or subjective parameters; including, but not limited to, the results of a physical examination, imaging, or a blood test. A “prophylactic” treatment is a treatment administered to a subject who does not exhibit signs of a disease or exhibits only early signs for the purpose of decreasing the risk of developing pathology, such as to prevent transplant rejection.

Isolated: An “isolated” biological molecule has been substantially separated, produced apart from, or purified away from other biological molecules in the cell of the organism in which the molecule naturally occurs, such as, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides, and proteins prepared by recombinant expression in a host cell as well as chemically synthesized nucleic acids.

Liver Disease: Diseases and conditions of the liver including liver cirrhosis, alcoholic and non-alcoholic fibrosis as well as to liver disease or changes associated with obesity, diabetes and metabolic syndrome. Other examples of liver diseases include: hepatitis, fatty liver, toxic liver failure, hepatic cirrhosis, diabetes-associated liver disease, liver steatosis, liver fibrosis, liver cirrhosis, chronic hepatitis and the like. Liver disease does not include liver cancer.

Liver regeneration: Morphologic changes in which hepatocyte growth occurs in either a recipient or a donor of a liver transplant. The hepatic growth generally results in an increase in hepatic function.

Liver transplantation: Partial and whole liver transplantations in which the liver of a donor is partially or wholly resected and partially or wholly transplanted into a recipient. In partial liver transplantation, a partial liver from a donor, corresponding to about 30-50% of the normal liver volume of a recipient, is harvested and grafted into a recipient. Grafting of one or more partial or whole hepatic tissue(s) or cell(s) can be taken or derived from another or the subject's own liver as a donor. A transplant can be autologous (from the same subject) or allogeneic (from a different subject) into a recipient. Generally, a liver transplant is allogeneic. The tissue can be matched for the Major Histocompatibility Complex (MHC) class II.

Lysophosphatidylcholine acyltransferase 3 (LPCAT3): A protein that catalyzes the reacylation step of the phospholipid remodeling process (the Lands cycle). It catalyzes transfer of the fatty acyl chain from fatty acyl-CoA to 1-acyl lysophospholipid to form various classes of phospholipids. LPCAT3 also converts 1-acyl lysophosphatidylcholine (LPC) into phosphatidylcholine (PC) (LPCAT activity), 1-acyl lysophosphatidylserine (LPS) into phosphatidylserine (PS) (LPSAT activity) and 1-acyl lysophosphatidylethanolamine (LPE) into phosphatidylethanolamine (PE) (LPEAT activity). It favors polyunsaturated fatty acyl-CoAs as acyl donors compared to saturated fatty acyl-CoAs and has higher activity for LPC acyl acceptors compared to LPEs and LPSs. Exemplary LPCAT3 nucleic acid and amino acid sequences are provided in GENBANK® Accession Nos. NM_005768 and NP_005759, respectively, as available on Nov. 12, 2021.

Macrovesicular Steatosis: Abnormal retention of lipids within a cell, reflecting an impairment of the normal processes of fatty acid and/or triglyceride synthesis and elimination. Excess lipid accumulates in vesicles that displace the cytoplasm. In macrovesicular steatosis, the vesicles become large enough to distort the cell's nucleus. The condition is not particularly detrimental to the cell in mild cases, large accumulations can disrupt cell constituents, and in severe cases cells may even burst. Many different mechanisms can disrupt normal lipid movement through the cell and cause steatosis. Those mechanisms can be classified based on whether they result in an oversupply of lipid or a failure of lipid breakdown. Oversupply of lipid may result from, among other conditions, obesity, insulin resistance, or alcoholism. Certain toxins, such as alcohols, carbon tetrachloride, aspirin, and diptheria toxin, among others, interfere with cellular machinery involved in lipid metabolism. In addition, certain metabolic diseases are characterized by defects in lipid metabolism. For example, in Gaucher's disease, the lysosomes fail to degrade glycolipids, resulting in steatosis.

Microvesicular Steatosis: A variant form of hepatic fat accumulation whose histologic features contrast with the much more common macrovesicular steatosis. The condition was originally described in association with conditions sharing a number of biochemical and clinical features: acute fatty liver of pregnancy, Reye's syndrome, Jamaican vomiting sickness, sodium valproate toxicity, high-dose tetracycline toxicity and certain congenital defects of urea cycle enzymes. Microvesicular steatosis has been observed in a wide variety of conditions, including alcoholism, toxicity of several medications, hepatitis delta virus infection (primarily in South America and Central Africa), sudden childhood death, congenital defects of fatty acid beta oxidation, cholesterol ester storage disease, Wolman disease and Alper's syndrome, see, e.g., M. L. Hautekeete et al., (1990) Acta Clin. Belg. 45(5):311-326.

Nanoparticle: A particle between 1 and 100 nanometers (nm) in size with a surrounding interfacial layer. The interfacial layer is an integral part of nanoscale matter, fundamentally affecting all of its properties. The interfacial layer typically consists of ions, inorganic and/or organic molecules.

Nuclear factor erythroid 2 like 2 (NFE2L2): A transcription factor that is a member of a family of basic leucine zipper (bZIP) proteins. NFE2L2 plays a role in the response to oxidative stress. For example, it binds to antioxidant response (ARE) elements present in the promoter region of many cytoprotective genes, such as phase 2 detoxifying enzymes, and promotes their expression, thereby neutralizing reactive electrophiles. Exemplary NFE2L2 nucleic acid sequences are provided in GENBANK® Accession Nos. NM_001145412, NM_001145413, NM_001313900, NM_001313901, NM_001313902, NM_001313903, NM_001313904, and NM_006164, as available on Nov. 12, 2021. Exemplary NFE2L2 amino acid sequences are provided in GENBANK® Accession Nos. NP_001138884, NP_001138885, NP_001300829, NP_001300830, NP_001300831, NP_001300832, NP_001300833, and NP_006155, as available on Nov. 12, 2021.

Nuclear receptor coactivator 4 (NCOA4): An androgen receptor coactivator. The encoded protein interacts with the androgen receptor in a ligand-dependent manner to enhance its transcriptional activity. It is also a ligand-independent coactivator of the peroxisome proliferator-activated receptor (PPAR) gamma. Exemplary NCOA4 nucleic acid sequences are provided in GENBANK® Accession Nos. NM_001145260, NM_001145261, NM_001145262, NM_001145263, and NM_005437, as available on Nov. 12, 2021. Exemplary NCOA4 amino acid sequences are provided in GENBANK® Accession Nos. NP_001138732, NP_001138733, NP_001138734, NP_001138735, and NP_005428, as available on Nov. 12, 2021.

Nucleic acid molecule: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see for example, Current Protocols in Molecular Biology (Ausubel et al., eds 1995 supplement)).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Patatin-like phospholipase domain-containing protein 3 (PNPLA3), and rs738409:G mutation: The PNPLA3 gene encodes a triacylglycerol lipase that mediates triacylglycerol hydrolysis in adipocytes. The highly conserved patatin-like domain of the encoded PNPLA3 protein exhibits lipolytic activity toward triglycerides. PNPLA3, which appears to be membrane bound, may be involved in the balance of energy usage/storage in adipocytes. PNPLA3 catalyzes coenzyme A (CoA)-dependent acylation of 1-acyl-sn-glycerol 3-phosphate (2-lysophosphatidic acid/LPA) to generate phosphatidic acid (PA), an important metabolic intermediate and precursor for both triglycerides and glycerophospholipids.

The rs738409 C>G polymorphism of PNPLA3, which encodes I148M, is strongly associated with hepatic fat content and confers susceptibility to nonalcoholic fatty liver disease (NAFLD). The PNPLA3 rs738409:G variant is also associated with fatty liver and alcoholic liver diseases, as well as fibrosis, histological disease severity, steatosis, and elevated levels of liver enzymes in healthy adults. The rs738409 variant is also a risk factor for cirrhosis (Shen et al. J Lipid Res. 56(1):167-175, 2015). Exemplary HMGCR nucleic acid and amino acid sequences are provided in GENBANK® Accession Nos. NM_025225 and NP_079501, respectively, as available on Nov. 12, 2021.

Partial Hepatectomy or Resection: A surgical procedure in which a portion of the liver is removed. The surgeon may remove a part of the liver, such as an entire lobe, or an even larger portion of the liver. In a partial hepatectomy, the surgeon typically leaves sufficient healthy liver tissue to maintain the functions of the liver in a donor.

Pharmaceutically acceptable carriers: The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or composition, including a nucleic acid molecule, capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

A promoter can be a constitutively active promoter (i.e., a promoter that is constitutively in an active/“ON” state), an inducible promoter (i.e., a promoter whose state, active/“ON” or inactive/“OFF”, is controlled by an external stimulus, e.g., the presence of a particular temperature, compound, or protein), a spatially restricted promoter (e.g., tissue specific promoter, cell type specific promoter, etc.), or it may be a temporally restricted promoter (i.e., the promoter is in the “ON” state or “OFF” state during specific stages of embryonic development or during specific stages of a biological process).

Purified: The term “purified” does not require absolute purity; rather, it is intended as a relative term. Thus, for example, a purified protein preparation is one in which the protein referred to is purer than the protein in its natural environment within a cell. For example, a preparation of a protein is purified such that the protein represents at least 50% of the total protein content of the preparation. Similarly, a purified nucleic acid molecule preparation is one in which the nucleic acid molecule is purer than in an environment including a complex mixture. A purified population of nucleic acids or proteins is greater than about 90%, 91%, 92%, 93%, 94%, 95%, 96%, 97%, 98%, 99% or 100% pure, or free other nucleic acids or proteins, respectively.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one coded for by a recombinant nucleic acid molecule.

Resection: The excision of a portion or all of an organ or other structure. For example, liver resection refers to surgical removal of a portion of the liver and is usually performed to remove the diseased portion of the liver, or to provide a portion of a liver for transplant into another subject.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a FGF polypeptide will possess a relatively high degree of sequence identity when aligned using standard methods.

Methods of alignment of sequences for comparison are well known in the art. Various programs and alignment algorithms are described in Smith and Waterman, Adv. Appl. Math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul, et al., Nature Genet., 6:119, 1994 presents a detailed consideration of sequence alignment methods and homology calculations.

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, such as version 7.0 (Devereaux et al., Nuc. Acids Res. 12:387-395, 1984.

As another example, the NCBI Basic Local Alignment Search Tool (BLAST) (Altschul, et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, sequence identity counted over the full-length alignment with the amino acid sequence of the factor using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Small-For-Size (SFS) Liver Transplant: A surgical technique in which a donor liver is split into two or more fragments, each of which is subsequently transplanted into a different recipient. Adequate hepatic regeneration is essential for recovery of patients receiving SFS transplants, most of whom are chronically ill with severely compromised liver function. Inadequate regeneration can result in “small-for-size graft syndrome,” characterized by poor bile production, intractable ascites, and prolonged cholestasis, and is often associated with surgical and septic complications.

Solute carrier family 1 member 5 (SLC1A5): A sodium-dependent amino acid transporter that has a broad substrate specificity, with a preference for zwitterionic amino acids. It accepts as substrates all neutral amino acids, including glutamine, asparagine, and branched-chain and aromatic amino acids, and excludes methylated, anionic, and cationic amino acids. Through binding of the fusogenic protein syncytin-1/ERVW-1, SLC1A5 may mediate trophoblast syncytialization, the spontaneous fusion of their plasma membranes, an essential process in placental development. Exemplary SLC1A5 nucleic acid sequences are provided in GENBANK® Accession Nos. NM_001145144, NM_001145145, and NM_005628, as available on Nov. 12, 2021. Exemplary SLC1A5 amino acid sequences are provided in GENBANK® Accession Nos. NP_001138616, NP_001138617, and NP_005619, as available on Nov. 12, 2021.

Solute carrier family 3 member 2 (SLC3A2): A member of the solute carrier family. SLC3A2 is a cell surface, transmembrane protein. The protein exists as the heavy chain of a heterodimer, covalently bound through di-sulfide bonds to one of several possible light chains. The encoded transporter plays a role in regulation of intracellular calcium levels and transports L-type amino acids. Exemplary SLC3A2 nucleic acid sequences are provided in GENBANK® Accession Nos. NM_001012662, NM_001012664, NM_001013251, and NM_002394, as available on Nov. 12, 2021. Exemplary SLC3A2 amino acid sequences are provided in GENBANK® Accession Nos. NP_001012680, NP_001012682, NP_001013269, and NP_002385, as available on Nov. 12, 2021.

Solute carrier family 7 member 11 (SLC7A11): A member of a heteromeric, sodium-independent, anionic amino acid transport system that is highly specific for cysteine and glutamate. In this system, designated Xc(−), the anionic form of cysteine is transported in exchange for glutamate. Exemplary SLC7A11 nucleic acid and amino acid sequences are provided in GENBANK® Accession Nos. NM_014331 and NP_055146, respectively, as available on Nov. 12, 2021.

Solute carrier family 33 member 1 (SLC33A1): A protein required for the formation of O-acetylated (Ac) gangliosides. SLC33A1 is predicted to contain 6 to 10 transmembrane domains, and a leucine zipper motif in transmembrane domain III. It is important for O-acetylation of gangliosides, and it negatively regulates BMP signaling. Exemplary SLC33A1 nucleic acid sequences are provided in GENBANK® Accession Nos. NM_001190992, NM_001363883, and NM_004733, as available on Nov. 12, 2021. Exemplary SLC33A1 amino acid sequences are provided in GENBANK® Accession Nos. NP_001177921, NP_001350812, and NP_004724, as available on Nov. 12, 2021.

Solute carrier family 38 member 1 (SLC38A1): A transporter of glutamine, an intermediate in the detoxification of ammonia and the production of urea. SLC38A1 functions as a sodium-dependent amino acid transporter. It mediates the saturable, pH-sensitive and electrogenic cotransport of glutamine and sodium ions with a stoichiometry of 1:1. Exemplary SLC38A1 nucleic acid sequences are provided in GENBANK® Accession Nos. NM_001077484, NM_001278387, NM_001278388, NM_001278389, NM_001278390, and NM_030674, as available on Nov. 12, 2021. Exemplary SLC38A1 amino acid sequences are provided in GENBANK® Accession Nos. NP_001070952, NP_001265316, NP_001265317, NP_001265318, NP_001265319, and NP_109599, as available on Nov. 12, 2021.

ST3 beta-galactoside alpha-2,3-sialyltransferase 2 (ST3GAL2): A type II membrane protein that catalyzes the transfer of sialic acid from CMP-sialic acid to galactose-containing substrates. ST3GAL2 is normally found in the Golgi but can be proteolytically processed to a soluble form. This protein, which is a member of glycosyltransferase family 29, can use the same acceptor substrates as does sialyltransferase 4A. ST3GAL2 is a beta-galactoside alpha2-3 sialyltransferase primarily involved in terminal sialylation of ganglio and globo series glycolipids. It catalyzes the transfer of sialic acid (N-acetyl-neuraminic acid; Neu5Ac) from the nucleotide sugar donor CMP-Neu5Ac onto acceptor Galbeta-(1->3)-GalNAc-terminated glycoconjugates through an alpha2-3 linkage. Exemplary ST3GAL2 nucleic acid and amino acid sequences are provided in GENBANK® Accession Nos. NM_006927 and NP_008858, respectively, as available on Nov. 12, 2021.

ST3 beta-galactoside alpha-2,3-sialyltransferase 5 (ST3GAL5): A type II membrane protein which catalyzes the formation of GM3 using lactosylceramide as the substrate. ST3GAL5 is a member of glycosyltransferase family 29 and may be localized to the Golgi apparatus. ST3GAL5 transfers the sialyl group (N-acetyl-alpha-neuraminyl or NeuAc) from CMP-NeuAc to the non-reducing terminal galactose (Gal) of glycosphingolipids forming gangliosides (important molecules involved in the regulation of multiple cellular processes, including cell proliferation and differentiation, apoptosis, embryogenesis, development, and oncogenesis). ST3GAL5 is mainly involved in the biosynthesis of ganglioside GM3 but can also use different glycolipids as substrate acceptors such as D-galactosylceramide (GalCer), asialo-GM2 (GA2) and asialo-GM1 (GA1), although less preferentially than beta-D-Gal-(1->4)-beta-D-Glc-(1<->1)-Cer (LacCer). Exemplary ST3GAL5 nucleic acid sequences are provided in GENBANK® Accession Nos. NM_001042437, NM_001354223, NM_001354224, NM_001354226, NM_001354227, NM_001354229, NM_001354233, NM_001354234, NM_001354238, NM_001354247, NM_001354248, NM_001363847, and NM_003896, as available on Nov. 12, 2021. Exemplary ST3GAL5 amino acid sequences are provided in GENBANK® Accession Nos. NP_001035902, NP_001341152, NP_001341153, NP_001341155, NP_001341156, NP_001341158, NP_001341162, NP_001341163, NP_001341167, NP_001341176, NP_001341177, NP_001350776, and NP_003887, as available on Nov. 12, 2021.

ST6 N-acetylgalactosaminide alpha-2,6-sialyltransferase 4 (ST6GALNAC4): A type II membrane protein that catalyzes the transfer of sialic acid from CMP-sialic acid to galactose-containing substrates. The encoded protein prefers glycoproteins rather than glycolipids as substrates and shows restricted substrate specificity, utilizing only the trisaccharide sequence Neu5Ac-alpha-2,3-Gal-beta-1,3-GalNAc. In addition, it is involved in the synthesis of ganglioside GD1A from GM1B. This protein is normally found in the Golgi apparatus but can be proteolytically processed to a soluble form.

ST6GALNAC4 is a member of glycosyltransferase family 29. Exemplary ST6GALNAC4 nucleic acid sequences are provided in GENBANK® Accession Nos. NM_175039 and NM_175040, as available on Nov. 12, 2021. Exemplary ST6GALNAC4 amino acid sequences are provided in GENBANK® Accession Nos. NP_778204 and NP_778205, as available on Nov. 12, 2021.

ST6 N-acetylgalactosaminide alpha-2,6-sialyltransferase 6 (ST6GALNAC6): A protein belonging to a family of sialyltransferases that modify proteins and ceramides on the cell surface to alter cell-cell or cell-extracellular matrix interactions. ST6GALNAC6 transfers the sialyl group (N-acetyl-alpha-neuraminyl or NeuAc) from CMP-NeuAc onto glycoproteins and glycolipids, forming an alpha-2,6-linkage. It produces branched type disialyl structures by transfer of a sialyl group onto the GalNAc or N-acetylglucosamine (GlcNAc) residue inside backbone core chains having a terminal sialic acid with an alpha-2,3-linkage on Gal. ST6GALNAC6 prefers glycolipids to glycoproteins, predominantly catalyzing the biosynthesis of ganglioside GDlalpha from GM1b. Besides GMb1, MSGG, and other glycolipids, it shows activity towards sialyl Lc4Cer generating disialyl Lc4Cer, which can lead to the synthesis of disialyl Lewis a (Le(a)). Exemplary ST6GALNAC6 nucleic acid sequences are provided in GENBANK® Accession Nos. NM_001286999, NM_001287000, NM_001287001, NM_001287002, NM_001287003, NM_001388489, and NM_013443, as available on Nov. 12, 2021. Exemplary ST6GALNAC6 amino acid sequences are provided in GENBANK® Accession Nos. NP_001273928, NP_001273929, NP_001273930, NP_001273931, NP_001273932, NP_001375418, and NP_038471, as available on Nov. 12, 2021.

Stearoyl CoA desaturase (SCD): An enzyme involved in lipid and fatty acid biosynthesis, primarily the synthesis of oleic acid. SCD belongs to the fatty acid desaturase family and is an integral membrane protein located in the endoplasmic reticulum. Stearoyl-CoA desaturase utilizes oxygen and electrons from reduced cytochrome b5 to introduce the first double bond into saturated fatty acyl-CoA substrates. It catalyzes the insertion of a cis double bond at the delta-9 position in fatty acyl-CoA substrates, including palmitoyl-CoA and stearoyl-CoA. SCD activity gives rise to a mixture of 16:1 and 18:1 unsaturated fatty acids. Exemplary SCD nucleic acid and amino acid sequences are provided in GENBANK® Accession Nos. NM_005063 and NP_005054, respectively, as available on Nov. 12, 2021.

Subject: Human and non-human animals, including all vertebrates, such as mammals and non-mammals, such as non-human primates, mice, rabbits, sheep, dogs, cats, horses, cows, chickens, amphibians, and reptiles. In many embodiments of the described methods, the subject is a human.

Transcription factor AP-2 gamma (TFAP2C): A sequence-specific DNA-binding transcription factor involved in the activation of several developmental genes. The protein can act as either a homodimer or heterodimer with other family members and is induced during retinoic acid-mediated differentiation. TFAP2C is a sequence-specific DNA-binding protein that interacts with inducible viral and cellular enhancer elements to regulate transcription of selected genes. AP-2 transcription factors bind to the consensus sequence 5′-GCCNNNGGC-3′ and activate genes involved in a large spectrum of important biological functions including proper eye, face, body wall, limb and neural tube development. They also suppress various genes, including cluster of differentiation 146 (CD146, also known as the melanoma cell adhesion molecule (MCAM) or cell surface glycoprotein MUC18)), CCAAT enhancer binding protein (CEBPA), and the MYC proto-oncogene. Exemplary TFAP2C nucleic acid and amino acid sequences are provided in GENBANK® Accession Nos. NM_003222 and NP_003213, respectively, as available on Nov. 12, 2021.

Transgene: An exogenous gene.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Virus: Microscopic infectious organism that reproduces inside living cells. A virus consists essentially of a core of a single nucleic acid surrounded by a protein coat and has the ability to replicate only inside a living cell. “Viral replication” is the production of additional virus by the occurrence of at least one viral life cycle. Viral vectors are known in the art, and include, for example, adenovirus, AAV, lentivirus and herpes virus.

Zinc finger E-box binding homeobox 1 (ZEB1): A zinc finger transcription factor ZEB1 enhances or represses the promoter activity of the ATPase Na+/K+ transporting subunit alpha 1 (ATP1A1) gene depending on the quantity of cDNA and on the cell type. It also represses the E-cadherin promoter and induces an epithelial-mesenchymal transition (EMT) by recruiting SWI/SNF related, matrix associated, actin dependent regulator of chromatin, subfamily A, member 4 (SMARCA4/BRG1. ZEB1 represses BCL6 transcription repressor (BCL6) transcription in the presence of the corepressor CTBP1, and positively regulates neuronal differentiation. It represses transcription by binding to the E box (5′-CANNTG-3′). Further, ZEB1 promotes tumorigenicity by repressing stemness-inhibiting microRNAs. Exemplary ZEB1 nucleic acid sequences are provided in GENBANK® Accession Nos. NM_001128128, NM_001174093, NM_001174094, NM_001174095, NM_001174096, NM_001323638, NM_001323641, NM_001323642, NM_001323643, NM_001323644, NM_001323645, NM_001323646, NM_001323647, NM_001323648, NM_001323649, NM_001323650, NM_001323651, NM_001323652, NM_001323653, NM_001323654, NM_001323655, NM_001323656, NM_001323657, NM_001323658, NM_001323659, NM_001323660, NM_001323661, NM_001323662, NM_001323663, NM_001323664, NM_001323665, NM_001323666, NM_001323671, NM_001323672, NM_001323673, NM_001323674, NM_001323675, NM_001323676, NM_001323677, NM_001323678, and NM_030751, as available on Nov. 12, 2021. Exemplary ZEB1 amino acid sequences are provided in GENBANK® Accession Nos. NP_001121600, NP_001167564, NP_001167565, NP_001167566, NP_001167567, NP_001310567, NP_001310570, NP_001310571, NP_001310572, NP_001310573, NP_001310574, NP_001310575, NP_001310576, NP_001310577, NP_001310578, NP_001310579, NP_001310580, NP_001310581, NP_001310582, NP_001310583, NP_001310584, NP_001310585, NP_001310586, NP_001310587, NP_001310588, NP_001310589, NP_001310590, NP_001310591, NP_001310592, NP_001310593, NP_001310594, NP_001310595, NP_001310600, NP_001310601, NP_001310602, NP_001310603, NP_001310604, NP_001310605, NP_001310606, NP_001310607, and NP_110378, as available on Nov. 12, 2021.

Although methods and materials similar or equivalent to those described herein can be used in the practice or testing of the present disclosure, suitable methods and materials are described below. The materials, methods, and examples are illustrative only and not intended to be limiting.

### Overview of Several Embodiments

Clause 1. A method of promoting survival of a donor liver in a recipient subject, wherein the donor liver is homozygous for rs738409:G mutation in the patatin-like phospholipase domain-containing protein 3 (PNPLA3) gene, the method comprising administering to the subject a therapeutically effective amount of
(a) a nucleic acid molecule encoding a component of the ferroptosis pathway, wherein the component is GPX4, GPX1, GPX3, GPX3, FSP1, SCD, FADS2, GLS2, NFE2L2, BACH1, CREB1, GRSF1, TFAP2C, HMGCR, GCK, or CYP1A1;
(b) a nucleic acid inhibitor of a component of the ferroptosis pathway, wherein the component is ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS;
(c) glutathione (GSH) or a nucleic acid molecule encoding an enzyme that increases glutathione production, wherein the enzyme is GLS2, SLC3A2, SLC7A11, SLC38A1, SLC1A5, or GSR; or
(d) a supplement that increases glutathione in the subject, wherein the supplement is at least one of Glutamine, Glutamate, GSSG, Cysteine, Glycine, Cys-Gly, Choline, Phosphatidylcholine, Sorbitol, Palmitoylcarnitine, Methylthioadenosine, S-Adenosylhomocysteine, S-Adenosylmethionine, Cystine, Methionine Creatine, Gamma-aminobutyrate, or Guanadinoacetate;
thereby promoting survival of the donor liver in the recipient subject.

Clause 2. A method of promoting survival of a donor liver in a recipient subject, wherein the donor liver is homozygous for rs738409:G mutation in the PNPLA3gene, the method comprising administering to the donor liver a therapeutically effective amount of:
(a) a nucleic acid molecule encoding a component of the ferroptosis pathway, wherein the component is GPX4, GPX1, GPX2, GPX3, FSP1, SCD, FADS2, GLS2, NFE2L2, BACH1, CREB1, GRSF1, TFAP2C, HMGCR, GCK, or CYP1A1; or
(b) a nucleic acid inhibitor of a component of the ferroptosis pathway, wherein the component is ACSL4, NCOA4, ATG5, LPCAT3, ZEB1-ST3GAL5, SLC33A1, ST3GAL2, ST6GALNAC4, HS3ST3A1, B4GALNT1, ST6GALNAC6, HEXB, HYAL4, or GNS;
thereby promoting survival of the donor liver in the recipient subject.

Clause 3. The method of clause 1 or clause 2, wherein the donor liver is from a donor, and wherein the method further comprises detecting the rs738409:G mutation in the PNPLA3 gene in a sample from the donor.

Clause 4. The method of clause 3, wherein the sample from the donor is a blood or tissue sample.

Clause 5. The method of clause 4, wherein the tissue sample is a liver sample.

Clause 6. The method of any one of clauses 1-5, wherein the subject is the recipient of a cadaveric liver transplant.

Clause 7. The method of any one of clauses 1-6, wherein the subject is the recipient of a liver transplant from a living donor.

Clause 8. The method of any one of clauses 1-7, comprising administering to the subject the therapeutically effective amount of the nucleic acid molecule encoding a component of the ferroptosis pathway.

Clause 9. The method of any one of clauses 2-7, comprising administering to the donor liver the therapeutically effective amount of the nucleic acid molecule encoding a component of the ferroptosis pathway in an ex vivo perfusion system.

Clause 10. The method of any one of clauses 1-9, wherein the nucleic acid molecule encoding a component of the ferroptosis pathway is an mRNA.

Clause 11. The method of clause 10, wherein the mRNA is administered to the subject or the donor liver using lipid nanoparticles (LNP), using polymeric nanoparticles, as a conjugate to GalNAc, as an mRNA modified by base linker sugars, using a degradable polymer, as an mRNA-Lipoplex, or as mRNA cargo of PEG-10.

Clause 12. The method of any one of clauses 1-11, wherein the nucleic acid molecule encoding a component of the ferroptosis pathway encodes GPX4.

Clause 13. The method of any one of clauses 1-11, wherein the nucleic acid molecule encoding a component of the ferroptosis pathway encodes GPX1, GPX2, GPX3, FSP1, SCD, FADS2, GLS2, NFE2L2, BACH1, CREB1, GRSF1, TFAP2C, HMGCR, GCK, or CYP1A1.

Clause 14. The method of any one of clauses 8-13, comprising administering to the subject or the donor liver a therapeutically effective amount of a viral vector comprising the nucleic acid molecule encoding a component of the ferroptosis pathway.

Clause 15. The method of clause 14, wherein the viral vector is a lentiviral vector.

Clause 16. The method of clause 14, wherein the viral vector is an adeno-associated virus (AAV) vector.

Clause 17. The method of clause 16, wherein the AAV vector is an AAV1, AAV2, AAV3, AAV4, AAV5, AAV6, AAV7, AAV9, AAV10, AAV11, or AAV12 vector, or a hybrid of two or more AAV serotypes.

Clause 18. The method of any one of clauses 1-7, comprising administering to the subject the therapeutically effective amount of the nucleic acid inhibitor of a component of the ferroptosis pathway.

Clause 19. The method of any one of clauses 1-7, comprising administering to the donor liver the therapeutically effective amount of the nucleic acid inhibitor of a component of the ferroptosis pathway in an ex vivo perfusion system.

Clause 20. The method of clause 18 or 19, wherein the nucleic acid inhibitor of a component of the ferroptosis pathway is a siRNA, a ribozyme, or a shRNA.

Clause 21. The method of any one of clauses 18-20, wherein the nucleic acid inhibitor of a component of the ferroptosis pathway downregulates ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS.

Clause 22. The method of any one of clauses 18-21, comprising administering to the subject or the donor liver a therapeutically effective amount of a viral vector comprising the nucleic acid inhibitor of a component of the ferroptosis pathway.

Clause 23. The method of clause 22, wherein the viral vector is a lentiviral vector.

Clause 24. The method of clause 22, wherein the viral vector is an adeno-associated virus (AAV) vector.

Clause 25. The method of clause 24, wherein the AAV vector is an AAV1, AAV2, AAV3, AAV4, AAV5, AAV6, AAV7, AAV9, AAV10, AAV11, or AAV12 vector, or a hybrid of two or more AAV serotypes.

Clause 26. The method of any one of clauses 1-7, comprising administering to the subject the therapeutically effective amount of the nucleic acid molecule encoding an enzyme that increases glutathione production.

Clause 27. The method of any one of clauses 1-7, comprising administering to the donor liver the therapeutically effective amount of the nucleic acid molecule encoding an enzyme that increases glutathione production in an ex vivo perfusion system.

Clause 28. The method of clause 26 or 27, wherein the nucleic acid molecule encoding an enzyme that increases glutathione production is an mRNA.

Clause 29. The method of any one of clauses 26-28, wherein the nucleic acid molecule encoding an enzyme that increases glutathione production encodes GLS2, SLC3A2, SLC7A11, SLC38A1, SLC1A5, or GSR.

Clause 30. The method of any one of clauses 26-29, comprising administering to the subject or the donor liver a therapeutically effective amount of a viral vector comprising the nucleic acid molecule encoding an enzyme that increases glutathione production.

Clause 31. The method of clause 30, wherein the viral vector is a lentiviral vector.

Clause 32. The method of clause 31, wherein the viral vector is an adeno-associated virus (AAV) vector.

Clause 33. The method of clause 32, wherein the AAV vector is an AAV1, AAV2, AAV3, AAV4, AAV5, AAV6, AAV7, AAV9, AAV10, AAV11, or AAV12 vector, or a hybrid of two or more AAV serotypes.

Clause 34. The method of any one of clauses 1-7, comprising administering to the subject the therapeutically effective amount of GSH.

Clause 35. The method of any one of clauses 1-7, comprising administering to the subject the therapeutically effective amount of one or more of Glutamine, Glutamate, GSSG, Cysteine, Glycine, Cys-Gly, Choline, Phosphatidylcholine, Sorbitol, Palmitoylcarnitine, Methylthioadenosine, S-Adenosylhomocysteine, S-Adenosylmethionine, Cystine, Methionine Creatine, Gamma-aminobutyrate, or Guanadinoacetate.

Clause 36. The method of any one of clauses 1-35, further comprising administering to the subject a therapeutically effective amount of deferoxamine (DFO), selenium, vitamin E (alpha-tocopherol), CoQ10, or a combination thereof.

Clause 37. The method of any one of clauses 1-35, wherein the method inhibits lipid peroxidation and/or mitochondrial shrinkage in donor liver cells, and/or increases survival of donor liver cells.

Clause 38. A composition comprising a nucleic acid molecule encoding GPX4 for use in the method of any one of clauses 1-17 or 36-37.

Clause 39. A composition comprising a nucleic acid molecule encoding GPX1, GPX2, GPX3, FSP1, SCD, FADS2, GLS2, NFE2L2, BACH1, CREB1, GRSF1, TFAP2C, HMGCR, GCK, or CYP1A1 for use in the method of any one of clauses 1-12, 13-17, or 36-37.

Clause 40. A composition comprising a nucleic acid inhibitor of a component of the ferroptosis pathway, wherein the component is ACSL4, NCOA4, ATG5, LPCAT3, ZEB1_ST3GAL5, SLC33A1, ST3GAL2, ST6GALNAC4, HS3ST3A1, B4GALNT1, ST6GALNAC6, HEXB, HYAL4, or GNS, for use in the method of any one of clauses 1-7, 18-25, or 36-37.

Clause 41. A composition comprising GSH for use in the method of any one of clauses 1-7, 34, or 36-37.

Clause 42. A composition comprising a nucleic acid molecule encoding GLS2, SLC3A2, SLC7A11, SLC38A1, SLC1A5, or GSR for use in the method of any one of clauses 1-7, 26-33, or 36-37.

Clause 43. A composition comprising at least one of Glutamine, Glutamate, GSSG, Cysteine, Glycine, Cys-Gly, Choline, Phosphatidylcholine, Sorbitol, Palmitoylcarnitine, Methylthioadenosine, S-Adenosylhomocysteine, S-Adenosylmethionine, Cystine, Methionine Creatine, Gamma-aminobutyrate, or Guanadinoacetate for use in the method of any one of clauses 1-7 or 35-37.

### Polynucleotides Encoding a Component of the Ferroptosis Pathway, Such as GPX4

Ferroptosis is a type of programmed cell death dependent on iron and characterized by the accumulation of lipid peroxides. Ferroptosis is initiated by the failure of the glutathione-dependent antioxidant defenses, resulting in unchecked lipid peroxidation and eventual cell death. It is disclosed herein that a nucleic acid molecule encoding a component of the ferroptosis pathway, such as GPX4, GPX1, GPX2, GPX3, FSP1, SCD, FADS2, GLS2, NFE2L2, BACH1, CREB1, GRSF1, TFAP2C, HMGCR, GCK, or CYP1A1, can be used to promote survival of a donor liver in a recipient subject, wherein the donor liver is homozygous for rs738409:G mutation in the PNPLA3 gene. It is also disclosed herein that a nucleic acid molecule encoding an enzyme that increases glutathione production, such as GLS2, SLC3A2, SLC7A11, SLC38A1, SLC1A5, or GSR, can be used to promote survival of a donor liver in a recipient subject, wherein the donor liver is homozygous for rs738409:G mutation in the PNPLA3 gene.

Polynucleotides include DNA, cDNA and RNA sequences which encode a protomer and a component of the ferroptosis pathway, as well as vectors including the DNA, cDNA, and RNA sequences, such as a DNA or RNA vector, are of use in the methods disclosed herein. The genetic code can be used to construct a variety of functionally equivalent nucleic acids, such as nucleic acids which differ in sequence but which encode the same protein sequence (degenerate variants). In a specific, non-limiting embodiment, the nucleic acid molecule encodes GPX4. In another specific, non-limiting embodiment, the nucleic acid molecule encodes a sequence having at least 80%, at least 85%, at least 90%, at least 95%, at least 96%, at least 97%, at least 98% or at least 99% identical to a nucleotide sequence set forth as SEQ ID NO: 3 or 4.

Polynucleotides encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein are of use in the disclosed methods. In some non-limiting examples, the polynucleotides encode GPX4. These polynucleotides include DNA, cDNA, and RNA sequences that encode the protein. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (e.g., L. Stryer, 1988, Biochemistry, 3.sup.rd Edition, W.H. 5 Freeman and Co., NY). Degenerate variants are also of use in the methods disclosed herein.

Nucleic acid molecules encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein can readily be produced by one of skill in the art using the amino acid sequences provided herein and the genetic code. Nucleic acid sequences encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984 and the solid support method of U.S. Pat. No. 4,458,066. Chemical synthesis produces a single-strand (ss) oligonucleotide, which can be converted into double-strand (ds) DNA by hybridization with a complementary sequence or by polymerization with a DNA polymerase using the single strand as a template. Exemplary nucleic acids that include sequences encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein can be prepared by cloning techniques.

A nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein can be cloned or amplified by in vitro methods, such as the polymerase chain reaction (PCR), the ligase chain reaction (LCR), the transcription-based amplification system (TAS), the self-sustained sequence replication system (3SR), and the QP replicase amplification system (QB). For example, a polynucleotide encoding the protein can be isolated by a polymerase chain reaction of cDNA using primers based on the DNA sequence of the molecule. A wide variety of cloning and in vitro amplification methodologies are well-known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

In some embodiments, a polynucleotide sequence encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein is operably linked to transcriptional control sequences including, for example a promoter and a polyadenylation signal. Any promoter can be used that is a polynucleotide sequence recognized by the transcriptional machinery of the host cell (or introduced synthetic machinery) that is involved in the initiation of transcription. A polyadenylation signal is a polynucleotide sequence that directs the addition of a series of nucleotides on the end of the mRNA transcript for proper processing and trafficking of the transcript out of the nucleus into the cytoplasm for translation.

Exemplary promoters include viral promoters, such as cytomegalovirus immediate early gene promoter (“CMV”), herpes simplex virus thymidine kinase (“tk”), SV40 early transcription unit, polyoma, retroviruses, papilloma virus, hepatitis B virus, and human and simian immunodeficiency viruses. Other promoters include promoters isolated from mammalian genes, such as the immunoglobulin heavy chain, immunoglobulin light chain, T cell receptor, HLA-DQα and HLA-DQβ, β-interferon, interleukin-2, interleukin-2 receptor, MHC class II, HLA-DRα, β-actin, muscle creatine kinase, prealbumin (transthyretin), elastase I, metallothionein, collagenase, albumin, fetoprotein, β-globin, c-fos, c-HA-ras, neural cell adhesion molecule (NCAM), al-antitrypsin, H2B (TH2B) histone, type I collagen, glucose-regulated proteins (GRP94 and GRP78), rat growth hormone, human serum amyloid A (SAA), troponin I (TNI), platelet-derived growth factor, and dystrophin, as well as promoters specific for liver cells.

The promoter can be either inducible or constitutive. An inducible promoter is a promoter that is inactive or exhibits low activity except in the presence of an inducer substance. Additional examples of promoters include, but are not limited to, MT II, MMTV, collagenase, stromelysin, SV40, murine MX gene, α-2-macroglobulin, MHC class I gene h-2kb, HSP70, proliferin, tetracycline inducible, tumor necrosis factor, or thyroid stimulating hormone gene promoter. One example of an inducible promoter is the interferon inducible ISG54 promoter (see Bluyssen et al., Proc. Natl Acad. Sci. 92: 5645-5649, 1995, herein incorporated by reference). In some embodiments, the promoter is a constitutive promoter that results in high levels of transcription upon introduction into a host cell in the absence of additional factors. The protein can be a liver specific promoter, such as an albumin promoter, α₁-antitrypsin (AAT) promoter (Serpina1), apolipoprotein E promoter, liver-specific promoter 1 (LP1), thyroxine-binding globulin (TBG) promoter, phosphoglycerate kinase 1 (PGK) promoter, cytochrome P450 2E1 (CYP2E1) promoter, α-fetoprotein (AFP) promoter, transthyretin (TTR) promoter, α₁-microglobulin enhancer, DC190 promoter, DC172 promoter, light strand promoter, liver-specific promoter (LSPs), hepatic control region-1 (HCR) promoter, liver-muscle promoter (LiMP), phosphoenolpyruvate carboxykinase (PEPCK) promoter, or hepatic nuclear factor-3 (HNF3) promoter.

Optionally, transcription control sequences include one or more enhancer elements, which are binding recognition sites for one or more transcription factors that increase transcription above that observed for the minimal promoter alone, and also be operably linked to the polynucleotide encoding the promoter and/or the nucleic acid molecule encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein. Regarding the nucleic acid molecule encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, introns can also be included that help stabilize mRNA and increase expression. This mRNA can then be isolated and used in the methods disclosed herein. In one embodiment, mRNA encoding GPX4 is used in the methods disclosed herein.

In some embodiments of the compositions and methods described herein, a nucleic acid sequence that encodes a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein is incorporated into a vector capable of expression in a host cell, using established molecular biology procedures. For example, nucleic acids, such as cDNAs, that encode a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein can be manipulated with standard procedures, such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate, or use of specific oligonucleotides in combination with PCR or other in vitro amplification. These vectors can include a promoter operably linked to a nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as the GPX4 protein.

Exemplary procedures sufficient to guide one of ordinary skill in the art through the production of a vector capable of expression in a host cell that includes a promoter, and/or a polynucleotide sequence encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein can be found, for example, in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

It may be desirable to include a polyadenylation signal to effect proper termination and polyadenylation of the gene transcript. Exemplary polyadenylation signals have been isolated from beta globin, bovine growth hormone, SV40, and the herpes simplex virus thymidine kinase genes.

The disclosed nucleic acid molecules can be included in a nanodispersion system, see, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006.

Dendrimers are synthetic three-dimensional macromolecules that are prepared in a step-wise fashion from simple branched monomer units, the nature and functionality of which can be easily controlled and varied. Dendrimers consist of an initiator core, surrounded by a layer of a selected polymer that is grafted to the core, forming a branched macromolecular complex. Dendrimers are typically produced using polymers such as poly(amidoamine) or poly(L-lysine). A dendrimer can be synthesized from the repeated addition of building blocks to a multifunctional core (divergent approach to synthesis), or towards a multifunctional core (convergent approach to synthesis) and each addition of a three-dimensional shell of building blocks leads to the formation of a higher generation of the dendrimers. Polypropylenimine dendrimers contain 100% protonable nitrogens and up to 64 terminal amino groups. Protonable groups are usually amine groups which are able to accept protons at neutral pH. For nucleic acid molecules, dendrimers can be formed from polyamidoamine and phosphorous containing compounds with a mixture of amine/amide or N—P(O₂)S as the conjugating units. Dendrimers of use for delivery of nucleic acid molecules is disclosed, for example, in PCT Publication No. 2003/033027, imported herein by reference.

The polynucleotides encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as a GPX4 protein, include a recombinant DNA which is incorporated into a vector in an autonomously replicating plasmid or virus or into the genomic DNA of a prokaryote or eukaryote, or which exists as a separate molecule (such as a cDNA) independent of other sequences. Viral vectors that include the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, can also be prepared. Numerous viral vectors are known in the art, including polyoma; SV40 (Madzak et al., 1992, J. Gen. Virol., 73:15331536); adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Nad. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256); vaccinia virus (Mackett et al., 1992, Biotechnology, 24:495-499); adeno-associated virus (Muzyczka, 1992, Curr. Top. Microbiol. Immunol., 158:91-123; On et al., 1990, Gene, 89:279-282); herpes viruses, including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199); Sindbis viruses (H. Herweijer et al., 1995, Human Gene Therapy 6:1161-1167; U.S. Pat. Nos. 5,091,309 and 5,2217,879); alphaviruses (S. Schlesinger, 1993, Trends Biotechnol. 11:18-22; I. Frolov et al., 1996, Proc. Natl. Acad. Sci. USA 93:11371-11377); and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art and may be obtained from commercial sources (such as PharMingen, San Diego, Calif.; Protein Sciences Corp., Meriden, Conn.; Stratagene, La Jolla, Calif.).

Thus, in one embodiment, the nucleic acid molecule encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as the GPX4 protein, is included in a viral vector. Suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors, lentivirus vectors and poliovirus vectors. Specific exemplary vectors are poxvirus vectors, such as vaccinia virus, fowlpox virus and a highly attenuated vaccinia virus (MVA), adenovirus, baculovirus, yeast, and the like. Adeno-associated virus vectors (AAV) are disclosed in additional detail below, and are of use in the disclosed methods.

Disclosed herein are methods and compositions that include utilizing one or more vectors, such as a viral vector, such as a retroviral vector, lentiviral vector, or an adenoviral vector, or an AAV vector that includes a nucleic acid molecule including a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as a GPX4 protein. Defective viruses, that entirely or almost entirely lack viral genes, can be used. The vector can be a lentiviral vector. Use of defective viral vectors allows for administration to specific cells without concern that the vector can infect other cells. The adenovirus vectors of use include replication competent, replication deficient, gutless forms thereof. The AAV vectors of use are replication deficient. Without being bound by theory, adenovirus vectors are known to exhibit strong expression in vitro, excellent titer, and the ability to transduce dividing and non-dividing cells in vivo (Hitt et al., Adv in Virus Res 55:479-505, 2000). When used in vivo these vectors lead to strong but transient gene expression due to immune responses elicited to the vector backbone. In some non-limiting examples, a vector of use is an attenuated adenovirus vector, such as the vector described by Stratford-Perricaudet et al. (J. Clin. Invest., 90:626-630 1992; La Salle et al., Science 259:988-990, 1993); or a defective AAV vector (Samulski et al., J. Virol., 61:3096-3101, 1987; Samulski et al., J. Virol., 63:3822-3828, 1989; Lebkowski et al., Mol. Cell. Biol., 8:3988-3996, 1988).

Recombinant AAV vectors are characterized in that they are capable of directing the expression and the production of the selected transgenic products in targeted cells. Thus, the recombinant vectors comprise at least all of the sequences of AAV essential for encapsidation and the physical structures for infection of target cells.

AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. In some embodiments, the AAV DNA includes a nucleic acid including a promoter operably linked to a nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as a GPX4 protein. Further provided are recombinant vectors, such as recombinant adenovirus vectors and recombinant adeno-associated virus (rAAV) vectors comprising a nucleic acid molecule(s) disclosed herein. In some embodiments, the AAV is rAAV8, and/or AAV2. However, the AAV serotype can be any other suitable AAV serotype, such as AAV1, AAV2, AAV3, AAV4, AAV5, AAV6, AAV7, AAV9, AAV10, AAV11 or AAV12, or a hybrid of two or more AAV serotypes.

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008). In some embodiments, these elements are included in the AAV vector.

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008). In some embodiments, these elements are included in the AAV vector.

AAV vectors can be used for gene therapy. Exemplary AAV of use are AAV2, AAV5, AAV6, AAV8 and AAV9. Adenovirus, AAV2 and AAV8 are capable of transducing cells in the liver. Thus, any of a rAAV2 or rAAV8 vector can be used in the methods disclosed herein. However, rAAV6 and rAAV9 vectors are also of use.

Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of an rAAV for the methods disclosed herein.

AAV possesses several additional desirable features for therapy, including the ability to bind and enter target cells, enter the nucleus, the ability to be expressed in the nucleus for a prolonged period of time, and low toxicity. AAV can be used to transfect cells, and suitable vector are known in the art, see for example, U.S. Published Patent Application No. 2014/0037585, incorporated herein by reference. Methods for producing rAAV suitable for gene therapy are well known in the art (see, for example, U.S. Published Patent Application Nos. 2012/0100606; 2012/0135515; 2011/0229971; and 2013/0072548; and Ghosh et al., Gene Ther 13(4):321-329, 2006), and can be utilized with the methods disclosed herein.

In some embodiments, the vector is a rAAV8 vector, a rAAV2 vector, a rAAV9 vector. In a specific non-limiting example, the vector is an AAV8 vector. AAV8 vectors are disclosed, for example, in U.S. Pat. No. 8,692,332, which is incorporated by reference herein. The location and sequence of the capsid, rep 68/78, rep 40/52, VP1, VP2 and VP3 are disclosed in this U.S. Pat. No. 8,692,332. The location and hypervariable regions of AAV8 are also provided. In some embodiments, the vector is an AAV2 variant vector, such as AAV7m8.

The vectors of use in the methods disclosed herein can contain nucleic acid sequences encoding an intact AAV capsid which may be from a single AAV serotype (e.g., AAV2, AAV6, AAV8 or AAV9). As disclosed in U.S. Pat. No. 8,692,332, vectors of use can also be recombinant, and thus can contain sequences encoding artificial capsids which contain one or more fragments of the AAV8 capsid fused to heterologous AAV or non-AAV capsid proteins (or fragments thereof). These artificial capsid proteins are selected from non-contiguous portions of the AAV2, AAV6, AAV8 or AAV9 capsid or from capsids of other AAV serotypes. For example, a rAAV vector may have a capsid protein comprising one or more of the AAV8 capsid regions selected from the VP2 and/or VP3, or from VP1, or fragments thereof selected from amino acids 1 to 184, amino acids 199 to 259; amino acids 274 to 446; amino acids 603 to 659; amino acids 670 to 706; amino acids 724 to 738 of the AAV8 capsid, which is presented as SEQ ID NO: 2 in U.S. Pat. No. 8,692,332. In another example, it may be desirable to alter the start codon of the VP3 protein to GTG. Alternatively, the rAAV may contain one or more of the AAV serotype 8 capsid protein hypervariable regions, for example aa 185-198; aa 260-273; aa447-477; aa495-602; aa660-669; and aa707-723 of the AAV8 capsid which is presented as SEQ ID NO: 2 in U.S. Pat. No. 8,692,332.

In some embodiments, a recombinant adeno-associated virus (rAAV) is generated having an AAV serotype 2 capsid. To produce the vector, a host cell which can be cultured that contains a nucleic acid sequence encoding an AAV serotype 2 capsid protein, or fragment thereof, as defined herein; a functional rep gene; a minigene composed of, at a minimum, AAV inverted terminal repeats (ITRs) and a transgene, such as encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein and sufficient helper functions to permit packaging in the AAV2 capsid protein. The biological molecules required to be cultured in the host cell to package an AAV minigene in an AAV capsid may be provided to the host cell in trans. Alternatively, any one or more of the required biological molecules (e.g., minigene, rep sequences, cap sequences, and/or helper functions) may be provided by a stable host cell which has been engineered to contain one or more of the required components using methods known to those of skill in the art. In some embodiments, a stable host cell will contain the required biological molecules s(s) under the control of an inducible promoter or a tissue specific promoter. Similar methods can be used to generate a rAAV2, rAAV8 or rAAV9 vector and/or virion.

A liver specific promoter can be included in the AAV vectors. In some embodiments, promoters include, but are not limited to, an A1AT promoter (α₁-antitrypsin, Serpina1), apolipoprotein E promoter, LP1 promoter, TBG promoter, PGK promoter, CYP2E1 promoter, Afp promoter, TTR promoter, α₁-microglobulin enhancer, DC190 promoter, DC172 promoter, LSP, liver-specific promoter, HCR promoter, LiMP promoter, PEPCK promoter, or HNF3 promoter.

In specific non-limiting examples, a liver-specific promoter, as disclosed above, is operably linked to a nucleic acid molecule encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as the GPX4 protein, and included in the AAV vector.

In other embodiments, a nucleic acid molecule encoding a component of the ferroptosis pathway, such as, but not limited to, a transgene encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as a GPX4 protein, can be under the control of a constitutive promoter. A non-limiting example of a suitable constitutive promoter is the cytomegalovirus promoter. Additional non-limiting examples are the ubiquitin or a chicken β-actin promoter. Promoters of use include liver specific promoters, such as an A1AT promoter (α₁-antitrypsin, Serpina1), apolipoprotein E promoter, LP1 promoter, TBG promoter, PGK promoter, CYP2E1 promoter, Afp promoter, TTR promoter, α₁-microglobulin enhancer, DC190 promoter, DC172 promoter, LSP, liver-specific promoter, HCR promoter, LiMP promoter, PEPCK promoter, or HNF3 promoter. In still another alternative, a selected stable host cell may contain selected component(s) under the control of a constitutive promoter and other selected component(s) under the control of one or more inducible promoters, such as for the production of rAAV in a packaging host cell. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing a rAAV can be delivered to the packaging host cell in the form of any genetic element which transfer the sequences carried thereon. The selected genetic element may be delivered by any suitable method, including those described herein. The methods used to construct vectors are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745. In some embodiments, elements of the selected AAV can be readily isolated using techniques available to those of skill in the art from an AAV serotype, including AAV8. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GENBANK®.

In another embodiment, an mRNA can be used to deliver a nucleic acid encoding GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR (for example GPX4) directly into cells. In some embodiments, nucleic acid-based vaccines based on mRNA may provide a potent alternative to the previously mentioned approaches. mRNA delivery precludes safety concerns about DNA integration into the host genome and can be directly translated in the host cell cytoplasm. Moreover, the simple cell-free, in vitro synthesis of RNA avoids the manufacturing complications associated with viral vectors. Two exemplary forms of RNA that can be used to deliver a nucleic acid include conventional non-amplifying mRNA (see, e.g., Petsch et al., “Protective efficacy of in vitro synthesized, specific mRNA vaccines against influenza A virus infection,” Nature biotechnology, 30(12):1210-6, 2012) and self-amplifying mRNA (see, e.g., Geall et al., “Nonviral delivery of self-amplifying RNA vaccines,” PNAS, 109(36): 14604-14609, 2012; Magini et al., “Self-Amplifying mRNA Vaccines Expressing Multiple Conserved Influenza Antigens Confer Protection against Homologous and Heterosubtypic Viral Challenge,” PLoS One, 11(8):e0161193, 2016; and Brito et al., “Self-amplifying mRNA vaccines,” Adv Genet., 89:179-233, 2015).

### Inhibitory Nucleic Acid Molecules Targeting the Activity of Ferroptosis Pathway Genes, Such as ACSL4, LPCAT3, NCOA4, or ATG5

Inhibitory nucleic acids that decrease the expression and/or activity of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS can also be used in the methods disclosed herein. In some examples, such inhibitor nucleic acid molecules decrease ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS expression or activity by at least 20%, at least 40%, at least 50%, at least 75%, at least 80%, at least 90%, at least 95%, at least 98% or even 100%. One embodiment is a RNA interference (RNAi), such as, but not limited to, small inhibitory RNA (siRNA) or short hairpin RNA, which can be used for interference or inhibition of expression of a target. RNAs that specifically target ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS are commercially available. Exemplary commercial RNAs include the following: ACSL4: Santa Cruz Biotechnology siRNA, shRNA (Catalog #sc-60619), Thermofisher (siRNA ID 122221, Catalog #AM51331); NCOA4: Santa Cruz Biotechnology siRNA, shRNA (Catalog #sc-29719), Thermofisher (siRNA ID 107703, Catalog #AM16708); ATG5: Santa Cruz Biotechnology siRNA, shRNA (Catalog #sc-41445), Thermofisher (siRNA ID 137766, Catalog #AM16708); LPCAT3: Santa Cruz Biotechnology siRNA, shRNA (Catalog #sc-95749), Thermofisher (siRNA ID 122584, Catalog #AM16708); ZEB1: Santa Cruz Biotechnology siRNA, shRNA (Catalog #sc-38643), Thermofisher (siRNA ID 109653, Catalog #AM16708); ST3GAL5: Santa Cruz Biotechnology siRNA, shRNA (Catalog #sc-72297), Thermofisher (siRNA ID 111399, Catalog #AM16708); SLC33A1: Santa Cruz Biotechnology siRNA, shRNA (Catalog #sc-72429), Thermofisher (siRNA ID 107508, Catalog #AM16708); ST3GAL2: Santa Cruz Biotechnology siRNA, shRNA (Catalog #sc-93118), Thermofisher (siRNA ID 100313, Catalog #AM16708); ST6GALNAC4: Santa Cruz Biotechnology siRNA, shRNA (Catalog #sc-63020), Thermofisher (siRNA ID 111875, Catalog #AM16708); HS3ST3A1: Santa Cruz Biotechnology siRNA, shRNA (Catalog #sc-146085), Thermofisher (siRNA ID 100071, Catalog #AM16708); B4GALNT1: Santa Cruz Biotechnology siRNA, shRNA (Catalog #sc-146085), Thermofisher (siRNA ID 10067, Catalog #AM16708); ST6GALNAC6: Santa Cruz Biotechnology siRNA, shRNA (Catalog #sc-63022), Thermofisher (siRNA ID 111845, Catalog #AM16708); HEXB: Santa Cruz Biotechnology siRNA, shRNA (Catalog #sc-60785), Thermofisher (siRNA ID 119078, Catalog #AM16708); HYAL4: Santa Cruz Biotechnology siRNA, shRNA (Catalog #sc-146116), Thermofisher (siRNA ID 119287, Catalog #AM16708); GNS: Santa Cruz Biotechnology siRNA, shRNA (Catalog #sc-145660), Thermofisher (siRNA ID 10841, Catalog #AM16708).

Generally, siRNAs are generated by the cleavage of relatively long double-stranded RNA molecules by Dicer or DCL enzymes (Zamore, Science, 296:1265-1269, 2002; Bernstein et al., Nature, 409:363-366, 2001). In animals and plants, siRNAs are assembled into RISC and guide the sequence specific ribonucleolytic activity of RISC, thereby resulting in the cleavage of mRNAs or other RNA target molecules in the cytoplasm. In the nucleus, siRNAs also guide heterochromatin-associated histone and DNA methylation, resulting in transcriptional silencing of individual genes or large chromatin domains.

The present disclosure provides RNA suitable for interference or inhibition of expression of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS which RNA includes double stranded RNA of about 19 to about 40 nucleotides with the sequence that is substantially identical to a portion of an mRNA or transcript of a target gene, such as ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS for which interference or inhibition of expression is desired. For purposes of this disclosure, a sequence of the RNA “substantially identical” to a specific portion of the mRNA or transcript of the target gene for which interference or inhibition of expression is desired differs by no more than about 30%, and in some embodiments no more than about 10% or no more than 5% from the specific portion of the mRNA or transcript of the target gene. In particular embodiments, the sequence of the RNA is exactly identical to a specific portion of the mRNA or transcript of the target gene (e.g., ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS).

Thus, siRNAs disclosed herein include double-stranded RNA of about 15 to about 40 nucleotides in length and a 3′ or 5′ overhang having a length of 0 to 5-nucleotides on each strand, wherein the sequence of the double stranded RNA is substantially identical to (see above) a portion of a mRNA or transcript of a nucleic acid encoding ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS. In particular examples, the double stranded RNA contains about 19 to about 25 nucleotides, for instance 20, 21, or 22 nucleotides substantially identical to a nucleic acid encoding ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS. In additional examples, the double stranded RNA contains about 19 to about 25 nucleotides 100% identical to a nucleic acid encoding ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS It should be not that in this context “about” refers to integer amounts only. In one example, “about” 20 nucleotides refers to a nucleotide of 19 to 21 nucleotides in length.

Regarding the overhang on the double-stranded RNA, the length of the overhang is independent between the two strands, in that the length of one overhang is not dependent on the length of the overhang on other strand. In specific examples, the length of the 3′ or 5′ overhang is 0-nucleotide on at least one strand, and in some cases it is 0-nucleotide on both strands (thus, a blunt dsRNA). In other examples, the length of the 3′ or 5′ overhang is 1-nucleotide to 5-nucleotides on at least one strand. More particularly, in some examples the length of the 3′ or 5′ overhang is 2-nucleotides on at least one strand, or 2-nucleotides on both strands. In particular examples, the dsRNA molecule has 3′ overhangs of 2-nucleotides on both strands.

Thus, in one particular provided RNA embodiment, the double-stranded RNA contains 20, 21, or 22 nucleotides, and the length of the 3′ overhang is 2-nucleotides on both strands. In embodiments of the RNAs provided herein, the double-stranded RNA contains about 40-60% adenine+uracil (AU) and about 60-40% guanine+cytosine (GC). More particularly, in specific examples the double-stranded RNA contains about 50% AU and about 50% GC.

Also described herein are RNAs that further include at least one modified ribonucleotide, for instance in the sense strand of the double-stranded RNA. In particular examples, the modified ribonucleotide is in the 3′ overhang of at least one strand, or more particularly in the 3′ overhang of the sense strand. It is contemplated that examples of modified ribonucleotides include ribonucleotides that include a detectable label (for instance, a fluorophore, such as rhodamine or FITC), a thiophosphate nucleotide analog, a deoxynucleotide (considered modified because the base molecule is ribonucleic acid), a 2′-fluorouracil, a 2′-aminouracil, a 2′-aminocytidine, a 4-thiouracil, a 5-bromouracil, a 5-iodouracil, a 5-(3-aminoallyl)-uracil, an inosine, or a 2′O-Me-nucleotide analog.

Antisense and ribozyme molecules for ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS are also of use in the method disclosed herein. Antisense nucleic acids are DNA or RNA molecules that are complementary to at least a portion of a specific mRNA molecule (Weintraub, Scientific American 262:40, 1990). In the cell, the antisense nucleic acids hybridize to the corresponding mRNA, forming a double-stranded molecule. The antisense nucleic acids interfere with the translation of the mRNA, since the cell will not translate an mRNA that is double-stranded. In one embodiment, antisense oligomers are about 15 nucleotides are used, since they are easily synthesized and are less likely to cause problems than larger molecules when introduced into the target cell producing ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS. The use of antisense methods to inhibit the in vitro translation of genes is known (see, for example, Marcus-Sakura, Anal. Biochem. 172:289, 1988).

Generally, the principle behind antisense technology is that an antisense compound hybridizes to a target nucleic acid and effects the modulation of gene expression activity, or function, such as transcription, translation or splicing. The modulation of gene expression can be achieved by, for example, target RNA degradation or occupancy-based inhibition. An example of modulation of target RNA function by degradation is RNase H-based degradation of the target RNA upon hybridization with a DNA-like antisense compound, such as an antisense oligonucleotide. Antisense oligonucleotides can also be used to modulate gene expression, such as splicing, by occupancy-based inhibition, such as by blocking access to splice sites. Antisense compounds provide sequence-specific target gene regulation. This sequence-specificity makes antisense compounds effective tools for the selective modulation of a target nucleic acid of interest, such as a nucleic acid encoding ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS.

In some embodiments, expression of one or more genes of the ferroptosis pathway, such as ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, is inhibited at least about 10%, at least about 25%, at least 50%, at least 75%, at least 80%, at least 85%, at least 90%, or at least 95% relative to a control. In a specific, non-limiting example, expression of ACSL4 is inhibited at least about 10%, at least about 25%, at least 50%, at least 75%, at least 80%, at least 85%, at least 90%, or at least 95% relative to a control.

Any type of antisense compound that specifically targets and regulates expression of the one or more genes of the ferroptosis pathway, such as ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, is contemplated for use with the disclosed methods. Such antisense compounds include single-stranded compounds, such as antisense oligonucleotides, and double-stranded compounds, including compounds with at least partial double-stranded structure, including siRNAs, miRNAs, shRNAs and ribozymes. An antisense molecule also can have flanking sequences (e.g., regulatory sequences). A ribozyme can have any general structure including, without limitation, hairpin, hammerhead, or axehead structures, provided the molecule cleaves RNA. In some embodiments, the subject or the donor liver (such as by ex vivo perfusion) is administered a therapeutically effective amount of a viral vector comprising the nucleic acid inhibitor of a component of the ferroptosis pathway. Further, gene silencing can be used to reduce the activity of genes of the ferroptosis pathway, such as ACSL4, NCOA4, ATG5, LPCAT3, ZEB1,_ST3GAL5, SLC33A1, ST3GAL2, ST6GALNAC4, HS3ST3A1, B4GALNT1, ST6GALNAC6, HEXB, HYAL4, or GNS.

Methods of designing, preparing and using antisense compounds that specifically target a nucleic acid molecule encoding one or more genes of the ferroptosis pathway, such as ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, are within the abilities of one of skill in the art. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions. For example, an antisense nucleic acid molecule can be chemically synthesized using naturally occurring nucleotides or variously modified nucleotides designed to increase the biological stability of the molecules or to increase the physical stability of the duplex formed between the antisense and sense nucleic acids, such as phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridin-e, 5-carboxymethylaminomethyluracil, dihydrouracil, beta-D-galactosylqueosine, inosine, amongst others.

Furthermore, sequences for genes of the ferroptosis pathway, such as ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, are publicly available. The specific GENBANK® Accession numbers listed herein are provided for reference only and are not intended to be limiting.

Antisense compounds specifically targeting one or more genes of the ferroptosis pathway, such as ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, can be prepared by designing compounds that are complementary to the ferroptosis pathway gene nucleotide sequence, such as, for example, ferroptosis pathway gene translational start site sequence, such as a ACSL4 gene translational start site sequence. Antisense compounds targeting one or more genes of the ferroptosis pathway, such as ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, need not be 100% complementary to the nucleic acid sequence of the gene or genes, in order to specifically hybridize and regulate expression of the one or more genes. For example, the antisense compound, or antisense strand of the compound if a double-stranded compound, can be at least 75%, at least 80%, at least 85%, at least 90%, at least 95%, at least 96%, at least 97%, at least 98%, at least 99% or 100% complementary to the nucleic acid sequence of the one or more genes of the ferroptosis pathway, such as ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS. Methods of screening antisense compounds for specificity are well known in the art (see, for example, U.S. Patent Application Publication No. 2003/0228689).

Ribozymes, which are RNA molecules possessing the ability to specifically cleave other single-stranded RNA in a manner analogous to DNA restriction endonucleases, are also of use. Through the modification of nucleotide sequences, which encode these RNAs, it is possible to engineer molecules that recognize specific nucleotide sequences in an RNA molecule and cleave it (Cech, J. Amer. Med. Assn. 260:3030, 1988). An advantage of this approach is that, because they are sequence-specific, only mRNAs with particular sequences are inactivated.

There are two basic types of ribozymes namely, tetrahymena-type (Hasselhoff, Nature 334:585, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-base recognition sequences are preferable to shorter recognition sequences.

Various delivery systems are known and can be used to administer the siRNAs and other inhibitory nucleic acid molecules as therapeutics. Such systems include, for example, encapsulation in liposomes, microparticles, microcapsules, nanoparticles, recombinant cells capable of expressing the therapeutic molecule(s) (see, e.g., Wu et al., J. Biol. Chem. 262, 4429, 1987), construction of a therapeutic nucleic acid as part of a retroviral or other vector, and the like.

### Nucleic Acid Molecules and Methods of their Administration

Pharmaceutical compositions that include a nucleic acid molecule encoding a component of the ferroptosis pathway, wherein the component is GPX4, GPX1, GPX2, GPX3, FSP1, SCD, FADS2, GLS2, NFE2L2, BACH1, CREB1, GRSF1, TFAP2C, HMGCR, GCK, or CYP1A1 are of use in the methods disclosed herein. Pharmaceutical compositions that include a nucleic acid molecule that is an inhibitor of a component of the ferroptosis pathway, wherein the component is ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS are also of use in the method disclosed herein. Pharmaceutical compositions that include a nucleic acid molecule encoding an enzyme that increases glutathione production, wherein the enzyme is GLS2, SLC3A2, SLC7A11, SLC38A1, SLC1A5, or GSR are of use in the methods disclosed herein. In a specific non-limiting example, a pharmaceutical composition including a nucleic acid molecule encoding GPX4 are of use in the methods disclosed herein.

The pharmaceutical compositions including a nucleic acid molecule can be formulated and administered in a variety of ways depending on the type of disease to be treated (see, e.g., U.S. Published Application No. 2005/0054567, which discloses pharmaceutical compositions as well as administration of such compositions and is incorporated herein by reference). The pharmaceutical compositions can include a nanoparticle or dendrimer. These pharmaceutical compositions are of use in the methods disclosed herein. In one non-limiting example, the pharmaceutical compositions include a nucleic acid molecule encoding GPX4.

Pharmaceutical compositions including a nucleic acid molecule are provided that are formulated for local delivery to the liver. The disclosure includes within its scope pharmaceutical compositions comprising a nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein. The disclosure also includes within its cope a nucleic acid molecule that can be used to express an inhibitor of a component of the ferroptosis pathway, wherein the component is ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS. The pharmaceutical composition can include a viral vector including a nucleic acid molecule including a promoter operably linked to a nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, or a nucleic acid molecule that can be used to express an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, for example a lentiviral vector or an AAV vector. In one non-limiting example, the pharmaceutical composition includes a lentiviral vector or an AAV vector that encodes GPX4. In another non-limiting example, the pharmaceutical composition includes a lentiviral vector or an AAV vector that encodes a nucleic acid molecule expressing an inhibitor of ACSL4.

The nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, or a nucleic acid molecule that is an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, can be administered ex vivo (such as into a donor liver in an ex vivo perfusion system) or in vivo to the subject, such as, but not limited to, oral, intravenous, or intrahepatic (such as via hepatic vein or artery) administration. In some embodiments, the nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, or a nucleic acid molecule that is an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS is administered to a living donor prior to transplantation. Generally, it is desirable to prepare the compositions as pharmaceutical compositions appropriate for the intended application. Accordingly, methods for making a pharmaceutical composition containing the nucleic acid molecules, or vectors described above, are included herein. Typically, preparation of a pharmaceutical composition entails preparing a pharmaceutical composition that is essentially free of pyrogens, as well as any other impurities that could be harmful to humans or animals. Typically, the pharmaceutical composition contains appropriate salts and buffers to render the composition stable and allow for uptake of nucleic acids or virus by target cells.

Pharmaceutical compositions including nucleic acid molecules can be formulated for injection, such as for intrahepatic or intravenous administration. Such compositions are formulated generally by mixing a disclosed nucleic acid molecule at the desired degree of purity in a unit dosage injectable form (solution, suspension, or emulsion) with a pharmaceutically acceptable carrier, for example, one that is non-toxic to recipients at the dosages and concentrations employed and is compatible with other ingredients of the formulation. Pharmaceutical compositions can include an effective amount of the nucleic acid molecule dispersed (for example, dissolved or suspended) in a pharmaceutically acceptable carrier or excipient. Pharmaceutically acceptable carriers and/or pharmaceutically acceptable excipients are known in the art and are described, for example, in Remington's Pharmaceutical Sciences by E. W. Martin, Mack Publishing Co., Easton, PA, 19th Edition (1995). The nature of the carrier will depend on the particular mode of administration being employed. For example, formulations usually contain injectable fluids that include pharmaceutically and physiologically acceptable fluids, such as water, physiological saline, balanced salt solutions, aqueous dextrose, glycerol, or the like, as a vehicle. In addition, pharmaceutical compositions to be administered can contain minor amounts of non-toxic auxiliary substances, such as wetting or emulsifying agents, preservatives, pH buffering agents and the like, for example, sodium acetate or sorbitan monolaurate. A disclosed nucleic acid molecule can be suspended in an aqueous carrier, for example, in an isotonic or hypotonic buffer solution at a pH of about 3.0 to about 8.5, such as about 4.0 to about 8.0, about 6.5 to about 8.5, or about 7.4. Useful buffers include saline-buffered phosphate or an ionic boric acid buffer. The active ingredient, optionally together with excipients, can also be in the form of a lyophilisate and can be made into a solution prior to administration by the addition of suitable solvents.

The pharmaceutically acceptable carriers include any and all solvents, dispersion media, coatings, isotonic and absorption delaying agents, and the like. The use of such media and agents for pharmaceutically active substances is well-known in the art. Supplementary active ingredients also can be incorporated into the compositions. For example, certain pharmaceutical compositions can include the vectors or viruses in water, mixed with a suitable surfactant, such as hydroxy-propylcellulose. Dispersions also can be prepared in glycerol, liquid polyethylene glycols, and mixtures thereof as well as in oils. Under ordinary conditions of storage and use, these preparations contain a preservative to prevent the growth of microorganisms. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

In some embodiments, the excipients confer a protective effect to a virus including the nucleic acid molecules, such as AAV virion or lentivirus virion, such that loss of AAV virions or lentivirus virions, as well as transduceability resulting from formulation procedures, packaging, storage, transport, and the like, is minimized. These excipient compositions are therefore considered “virion-stabilizing” in the sense that they provide higher virion titers and higher transduceability levels than their non-protected counterparts, as measured using standard assays, see, for example, Published U.S. Application No. 2012/0219528, incorporated herein by reference. These compositions therefore demonstrate “enhanced transduceability levels” as compared to compositions lacking the particular excipients described herein and are therefore more stable than their non-protected counterparts.

Exemplary excipients that can used to protect a virion from activity degradative conditions include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate, preferably, sorbitol. The detergent, when present, can be an anionic, a cationic, a zwitterionic or a nonionic detergent. An exemplary detergent is a nonionic detergent. One suitable type of nonionic detergent is a sorbitan ester, e.g., polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85), such as TWEEN®-20 and/or TWEEN®-80. These excipients are commercially available from a number of vendors, such as Sigma, St. Louis, Mo.

The amount of the various excipients in any of the disclosed compositions including virus, such as AAV, varies and is readily determined by one of skill in the art. For example, a protein excipient, such as BSA, if present, will can be present at a concentration of between 1.0 weight (wt.) % to about 20 wt. %, such as 10 wt. %. If an amino acid such as glycine is used in the formulations, it can be present at a concentration of about 1 wt. % to about 5 wt. %. A carbohydrate, such as sorbitol, if present, can be present at a concentration of about 0.1 wt % to about 10 wt. %, such as between about 0.5 wt. % to about 15 wt. %, or about 1 wt. % to about 5 wt. %. If polyethylene glycol is present, it can generally be present on the order of about 2 wt. % to about 40 wt. %, such as about 10 wt. % top about 25 wt. %. If propylene glycol is used in the subject formulations, it will typically be present at a concentration of about 2 wt. % to about 60 wt. %, such as about 5 wt. % to about 30 wt. %. If a detergent such as a sorbitan ester (TWEEN®) is present, it can be present at a concentration of about 0.05 wt. % to about 5 wt. %, such as between about 0.1 wt. % and about 1 wt %, see U.S. Published Patent Application No. 2012/0219528, which is incorporated herein by reference. In one example, an aqueous virion-stabilizing formulation comprises a carbohydrate, such as sorbitol, at a concentration of between 0.1 wt. % to about 10 wt. %, such as between about 1 wt. % to about 5 wt. %, and a detergent, such as a sorbitan ester (TWEEN®) at a concentration of between about 0.05 wt. % and about 5 wt. %, such as between about 0.1 wt. % and about 1 wt. %. Virions are generally present in the composition in an amount sufficient to provide a therapeutic effect when given in one or more doses, as defined above.

The pharmaceutical compositions that include a nucleic acid molecule including a nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as a viral vector, or a pharmaceutical composition that includes a nucleic acid molecule that is an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, will, in some embodiments, be formulated in unit dosage form, suitable for individual administration of precise dosages. The amount of active compound(s) administered will depend on the subject being treated, the severity of the affliction, and the manner of administration and is best left to the judgment of the prescribing clinician. Within these bounds, the formulation to be administered will contain a quantity of the active compound(s) in amounts effective to achieve the desired effect in the subject being treated.

The nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as a viral vector or mRNA, or a nucleic acid molecule that is an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, can be included in an inert matrix for injection into the liver. As one example of an inert matrix, liposomes may be prepared from dipalmitoyl phosphatidylcholine (DPPC), such as egg phosphatidylcholine (PC). Liposomes, including cationic and anionic liposomes, can be made using standard procedures as known to one skilled in the art. For some applications, liposomes that include a nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein can be injected intrahepatically. In a specific non-limiting example, a nucleic acid molecule encoding GPX4 is formulated for intraheptic injection.

In other application, liposomes that include a nucleic acid molecule that is an inhibitor of a component of the ferroptosis pathway, wherein the component is ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS can be injected intrahepatically. In a formulation for intrahepatic injection, the liposome capsule degrades due to cellular digestion. Without being bound by theory, these formulations provide the advantages of a slow-release drug delivery system, exposing a subject to a substantially constant concentration of nucleic acid molecule over time. In one example, the nucleic acid molecule can be dissolved in an organic solvent, such as DMSO or alcohol, as previously described, and contain a polyanhydride, poly(glycolic) acid, poly(lactic) acid, or polycaprolactone polymer.

The nucleic acid molecule may be formulated to permit release over a specific period of time. A release system can include a matrix of a biodegradable material or a material which releases the incorporated nucleic acid molecule by diffusion. The nucleic acid molecule can be homogeneously or heterogeneously distributed within the release system. A variety of release systems may be useful; however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that active ingredients having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers, and mixtures thereof.

Poly(lactide-co-glycolide) microspheres can also be used for intrahepatic injection. Typically the microspheres are composed of a polymer of lactic acid and glycolic acid, which are structured to form hollow spheres. The spheres can be approximately 15-30 microns in diameter and can be loaded with the biologicla molecules described herein.

The nucleic acid molecule including a liver specific promoter operably linked to a nucleic acid molecule encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, such as in a viral vector or mRNA, can be included in a delivery system that can be implanted at various sites in the liver, depending on the size, shape, and formulation of the implant as well as the type of transplant procedure. Similarly, the nucleic acid molecule that is an inhibitor of a component of the ferroptosis pathway, wherein the component is ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS can be included in a delivery system that can be implanted at various sites in the liver.

The implants can be inserted into the liver by a variety of methods, which can influence the release kinetics. The location of the implanted device may influence the concentration gradients of the nucleic acid molecule surrounding the device and, thus, influence the release rates. Generally, when implants are used, the nucleic acid molecule is homogeneously distributed through the polymeric matrix, such that it is distributed evenly enough that no detrimental fluctuations in rate of release occur due to uneven distribution in the polymer matrix. The selection of the polymeric composition to be employed varies with the desired release kinetics, the location of the implant, patient tolerance, and the nature of the implant procedure. The polymer can be included as at least about 10 weight percent of the implant. In one example, the polymer is included as at least about 20 weight percent of the implant. In another embodiment, the implant comprises more than one polymer. These factors are described in detail in U.S. Pat. No. 6,699,493. Characteristics of the polymers can include biodegradability at the site of implantation, compatibility with the agent of interest, ease of encapsulation, and water insolubility, among others. Generally, the polymeric matrix is not fully degraded until the drug load has been released. The chemical composition of suitable polymers is known in the art (for example, see U.S. Pat. No. 6,699,493). The nucleic acid molecule can be formulated in an implantable form with other carriers and solvents. For example, buffering agents and preservatives can be employed. The implant sizes and shape can also be varied for use in particular regions of the liver (see U.S. Pat. No. 5,869,079). In some embodiments, a nanoparticle or dendrimer is used.

Local modes of administration include intrahepatic routes, such as administration to a donor liver prior to transplantation, or administration to the donor liver at the time of transplantation. In an embodiment, significantly smaller amounts (compared with systemic approaches) may exert an effect when administered locally (for example, intrahepatically) compared to when administered systemically (for example, intravenously). Local modes of administration can reduce or eliminate the incidence of potential side effects. Methods for administration of nucleic acid molecules to the liver are known in the medical arts and can be used in the methods described herein.

Administration may be provided as a single administration, a periodic bolus (for example, intrahepatically) or as continuous infusion from an internal reservoir (for example, from an implant disposed at an intrahepatic location or from an external reservoir (for example, from an intravenous bag). Intrahepatic injection of the nucleic acid molecules disclosed herein can be performed once, or can be performed repeatedly, such as 1, 2, 3, 4, 5, 6, 7, 8, 9, 10, 20, 30, 40, 50, 60, 70, 80, 90, or 100 or more times. Administration can be performed biweekly, weekly, every other week, monthly, or every 2, 3, 4, 5, or 6 months. In some embodiments, the nucleic acid molecule encodes GPX4.

Individual doses are typically not less than an amount required to produce a measurable effect on the subject and may be determined based on the pharmacokinetics and pharmacology of the subject composition or its by-products, and thus based on the disposition of the composition within the subject. This includes consideration of the route of administration as well as dosage amount, which can be adjusted for intravenous or intrahepatic applications. Effective amounts of dose and/or dose regimen can readily be determined empirically from preclinical assays, from safety and escalation and dose range trials, individual clinician-patient relationships, as well as in vitro and in vivo assays.

The nucleic acid molecule can be used alone. However, in another embodiment, at least one additional agent can be included along with the nucleic acid molecule in the implant. The implant is then introduced into the liver at one or more suitable sites. In one embodiment, the nucleic acid molecule encodes GPX4. In other embodiments, the additional agent is GSH.

Nucleic acid molecules can be delivered, such as to a recipient and/or to a donor liver (such as to a living donor prior to transplantation or to a donor liver in an ex vivo perfusion system), by microinjection, electroporation, lipid-mediated transfection, peptide-mediated delivery, nanoparticle mediated delivery (such as lipid or polymeric nanoparticle mediate delivery), dendrimer mediated delivery, as a conjugate to GalNAc, as an mRNA modified by base linker sugars, in association with a degradable polymer, as an mRNA-Lipoplex, as mRNA cargo of PEG-10, or other methods known in the art. In some embodiments, the mRNA, such as an mRNA encoding GPX4, is administered to the recipient and/or to the donor liver using lipid nanoparticles (LNP), using polymeric nanoparticles, as a conjugate to GalNAc, as an mRNA modified by base linker sugars, using a degradable polymer, as an mRNA-Lipoplex, or as mRNA cargo of PEG-10. In a specific, non-limiting example, the mRNA, such as an mRNA encoding GPX4, is administered to the recipient and/or to a donor liver using lipid nanoparticles. An appropriate dose depends on the subject being treated (e.g., human or nonhuman primate or other mammal), age and general condition of the subject to be treated, the severity of the condition being treated, the mode of administration, among other factors. An appropriate effective amount can be readily determined by one of skill in the art. Thus, a “therapeutically effective amount” will fall in a relatively broad range that can be determined through clinical trials.

In another embodiment, an mRNA-based administration protocol can be used to deliver a nucleic acid molecule encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein or a nucleic acid molecule that is an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, to promote survival of a donor liver in a recipient subject. mRNA deliver vehicles preclude safety concerns about DNA integration into the host genome and can be directly translated in the host cell cytoplasm. Moreover, cell-free, in vitro synthesis of RNA avoids the manufacturing complications associated with viral vectors.

In some embodiments, mRNA delivery is achieved using mRNA encoding a GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein or a nucleic acid molecule that is an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS as described herein and formulated as a lipid nanoparticle according to methods such as those described in WO2021154763, US20210228707, WO2017070626 and US2019/0192646, which are incorporated by reference herein. See, also, Jackson et al., N Engl J Med., 383(20):1920-1921, 2020, incorporated by reference herein. For example, the mRNA component can be a modified mRNA with 1-methylpseudouridine in place of uridine and a 7mG(5′)ppp(5′)N1mpNp cap. The mRNA sequence includes a 5′ untranslated region (UTR), a sequence encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein or a nucleic acid molecule that is an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, a 3′ UTR, and a polyA tail. In some embodiments, the sequence encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein or a nucleic acid molecule that is an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS is codon optimized relative to native sequence for mRNA expression in a human and to increase stability.

In several embodiments, the mRNA is formulated in a lipid nanoparticle for administration to the recipient or donor liver (such as to a living donor prior to transplantation or to the donor liver in an ex vivo perfusion system); for example, comprising a PEG-modified lipid, a non-cationic lipid, a sterol, an ionizable lipid, or any combination thereof. In some embodiments, the lipid nanoparticle is composed of 50 mol % ionizable lipid ((2 hydroxyethyl)(6 oxo 6-(undecycloxy)hexyl)amino)octanoate, 10 mol % 1,2 distearoyl sn glycerol-3 phosphocholine (DSPC), 38.5 mol % cholesterol, and 1.5 mol % 1-monomethoxypolyethyleneglycol-2,3, dimyristylglycerol with polyethylene glycol of average molecular weight 2000 (PEG2000 DMG). The mRNA/lipid nanoparticle composition may be provided in any suitable carrier, such as a sterile liquid for injection at a concentration of 0.5 mg/mL in 20 mM trometamol (Tris) buffer containing 87 mg/mL sucrose and 10.7 mM sodium acetate, at pH 7.5 and with appropriate diluent.

In some embodiments, for in vivo injection of a virion comprising a nucleic acid molecule encoding the GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein or a nucleic acid molecule that is an inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS, i.e., injection of a virion directly to the recipient or to the donor prior to liver transplantation, a therapeutically effective dose will be on the order of from about 10⁵ to 10¹⁶ of virions (such as AAV virions), such as 10⁸ to 10¹⁴ virions. The dose depends on the efficiency of transduction, promoter strength, the stability of the message and the protein encoded thereby, and clinical factors.

Effective dosages can be readily established by one of ordinary skill in the art through routine trials establishing dose response curves.

In some embodiments, if the nucleic acid molecule is included in an AAV vector, an effective amount will be about 1×10⁸ vector genomes or more, in some cases about 1×10⁹, about 1×10¹⁰, about 1×10¹¹, about 1×10¹², or about 1×10¹³ vector genomes or more, in certain instances, about 1×10¹⁴ vector genomes or more, and usually no more than about 1×10¹⁵ vector genomes administered to the recipient. In some embodiments, the amount of vector that is delivered is about 1×10¹⁴ vectors or less, for example about 1×10¹³, about 1×10¹², about 1×10¹¹, about 1×10¹⁰, or about 1×10⁹ vectors or less, in certain instances about 1×10⁸ vectors, and typically no less than 1×10⁸ vectors administered to the recipient. In some non-limiting examples, the amount of vector genomes that is delivered is about 1×10¹⁰ to about 1×10¹¹ vectors. In additional non-limiting examples, the amount of vector that is delivered is about 1×10¹⁰ to about 1×10¹² vector genomes.

In some embodiments, the amount of pharmaceutical composition to be administered may be measured using multiplicity of infection (MOI). In some embodiments, MOI refers to the ratio, or multiple of vector or viral genomes to the cells to which the nucleic may be delivered. In some embodiments, the MOI may be about 1×10⁶. In some cases, the MOI can be about 1×10⁵ to about 1×10⁷. In some cases, the MOI may be about 1×10⁴ to about 1×10⁸. In some cases, recombinant viruses of the disclosure are at least about 1×10¹, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10³, about 1×10¹⁴, about 1×10¹⁵, about 1×10¹⁶, about 1×10¹⁷, and about 1×10¹⁸ MOI. In some cases, recombinant viruses of this disclosure are about 1×10⁸ to 1×10¹⁴ MOI. In some embodiments, the dose is administered to the recipient. In other embodiments, the dose is administered to the donor organ prior to transplant.

In some the amount of pharmaceutical composition delivered comprises about 1×10⁸ to about 1×10¹⁵ particles of recombinant viruses, about 1×10⁹ to about 1×10¹⁴ particles of recombinant viruses, about 1×10¹⁰ to about 1×10¹³ particles of recombinant viruses, or about 1×10¹¹ to about 1×10¹² particles of recombinant viruses. These can be administered to the recipient. In other embodiments, the dose is administered to the donor organ prior to transplant.

Dosage treatment may be a single dose schedule or a multiple dose schedule to ultimately deliver the amount specified above. Moreover, the subject may be administered as many doses as appropriate. Thus, the recipient may be given, e.g., 10 s to 10¹⁶ AAV virions in a single dose, or two, four, five, six or more doses that collectively result in delivery of, e.g., 10⁵ to 10¹⁶ AAV virions. One of skill in the art can readily determine an appropriate number of doses to administer.

In some embodiments, an AAV is administered to the recipient and/or to the donor liver (such as in an ex vivo perfusion system) at a dose of about 1×10¹¹ to about 1×10¹⁴ viral particles (vp)/kg. In some examples, the AAV is administered to the recipient at a dose of about 1×10¹² to about 8×10¹³ vp/kg. In other examples, the AAV is administered to the recipient or to the donor liver at a dose of about 1×10¹³ to about 6×10¹³ vp/kg. In specific non-limiting examples, the AAV is administered to the recipient or to the donor liver at a dose of at least about 1×10¹¹, at least about 5×10¹¹, at least about 1×10¹², at least about 5×10¹², at least about 1×10¹³, at least about 5×10¹³, or at least about 1×10¹⁴ vp/kg. In other non-limiting examples, the AAV is administered to the recipient or to the donor liver at a dose of no more than about 5×10¹¹, no more than about 1×10¹², no more than about 5×10¹², no more than about 1×10¹³, no more than about 5×10¹³, or no more than about 1×10¹⁴ vp/kg. In one non-limiting example, the AAV is administered to the recipient or to the donor liver at a dose of about 1×10¹² vp/kg. The AAV can be administered in a single dose, or in multiple doses (such as 2, 3, 4, 5, 6, 7, 8, 9 or 10 or more doses) as needed for the desired therapeutic results.

In some embodiments, a lentivirus is administered to the recipient and/or to the donor liver (such as in an ex vivo perfusion system) at a dose of about 1×10¹¹ to about 1×10¹⁴ viral particles (vp)/kg. In some examples, the lentivirus is administered to the recipient or to the donor liver at a dose of about 1×10¹² to about 8×10¹³ vp/kg. In other examples, the lentivirus is administered to the recipient or to the donor liver at a dose of about 1×10¹³ to about 6×10¹³ vp/kg. In specific non-limiting examples, the lentivirus is administered to the recipient or to the donor liver at a dose of at least about 1×10¹¹, at least about 5×10¹¹, at least about 1×10¹², at least about 5×10¹², at least about 1×10³, at least about 5×10³, or at least about 1×10¹⁴ vp/kg. In other non-limiting examples, the lentivirus is administered to the recipient or to the donor liver at a dose of no more than about 5×10¹¹, no more than about 1×10¹², no more than about 5×10¹², no more than about 1×10¹³, no more than about 5×10¹³, or no more than about 1×10¹⁴ vp/kg. In one non-limiting example, the lentivirus is administered to the recipient or to the donor liver at a dose of about 1×10¹² vp/kg. The lentivirus can be administered in a single dose, or in multiple doses (such as 2, 3, 4, 5, 6, 7, 8, 9 or 10 or more doses) as needed for the desired therapeutic results.

In some embodiments of the disclosed methods, a therapeutically effective amount of a nucleic acid molecule encoding GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR, or a nucleic acid inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS is administered to the donor liver prior to transplantation of the liver into the recipient subject, such as by ex vivo perfusion. In particular, non-limiting examples, the donor liver is administered a therapeutically effective amount of a nucleic acid molecule encoding GPX4, thereby promoting survival of the donor liver in the recipient subject. Perfusion is the passage of fluid through the circulatory system or lymphatic system to an organ or a tissue, often referring to the delivery of blood, commonly with additives meant to prevent tissue damage prior to transplantation of the organ, to a capillary bed in tissue. Perfusion is measured as the rate at which fluid is delivered to tissue, or volume of fluid per unit time (e.g., blood flow) per unit tissue mass. For human organs perfusion is typically reported in ml/min/g.

Ex vivo perfusion systems are intended to mitigate ischemic injury during organ preservation and can also be used to deliver therapeutics to an organ after removal of the organ from a donor and prior to transplantation of the organ into a recipient. Clinically, these systems may replace the cold static storage preservation strategy for solid organ transplant. A number of donor liver ex vivo perfusion techniques are available as known to one of ordinary skill in the art, including hypothermic machine perfusion, subnormothermic perfusion, and normothermic perfusion. Through perfusion at different temperatures, ranging from hypothermic (+4 to +10° C.) to subnormothermic (+15° C. to 30° C.) and to normothermic (37° C.), graft metabolites can be flushed, nutrient supply optimized, and microvascular circulation maintained (van Beekum et al. Ann Transplant. 26: e931664-1-e931664-8, 2021). Concerning possible therapies, including gene therapy, this type of perfusion storage allows for intra delivery of high concentrations of, for example, mRNAs, miRNAs, siRNAs, shRNAs, and/or viral vectors, with continuous recirculation under metabolically favorable conditions.

The disclosed methods can include use of one more liver ex vivo perfusion systems as described herein and as known to one of ordinary skill in the art. In some embodiments, a donor liver is administered, in an ex vivo perfusion system, a therapeutically effective amount of a nucleic acid molecule encoding a component of the ferrotopsis pathway, such as, for example, GPX4, GPX1, GPX2, GPX3, FSP1, SCD, FADS2, GLS2, NFE2L2, BACH1, CREB1, GRSF1, TFAP2C, HMGCR, GCK, or CYP1A1. In particular embodiments, the nucleic acid molecule encodes GPX4. In some embodiments, the nucleic acid molecule encoding a component of the ferroptosis pathway is an mRNA. In some embodiments, the mRNA, such as an mRNA encoding GPX4, is administered, in an ex vivo perfusion system, to the donor liver using lipid nanoparticles (LNP), using polymeric nanoparticles, as a conjugate to GalNAc, as an mRNA modified by base linker sugars, using a degradable polymer, as an mRNA-Lipoplex, or as mRNA cargo of PEG-10. In a specific, non-limiting example, the nucleic acid molecule encoding a component of the ferrotopsis pathway, such as, for example, GPX4, GPX1, GPX2, GPX3, FSP1, SCD, FADS2, GLS2, NFE2L2, BACH1, CREB1, GRSF1, TFAP2C, HMGCR, GCK, or CYP1A1, is administered to the donor liver using lipid nanoparticles. In certain embodiments, the donor liver is administered, in an ex vivo perfusion system, a therapeutically effective amount of a viral vector comprising the nucleic acid molecule encoding a component of the ferroptosis pathway, such as a viral vector comprising a nucleic acid molecule encoding GPX4.

In other embodiments, a donor liver is administered, in an ex vivo perfusion system, a therapeutically effective amount of a nucleic acid inhibitor of a component of the ferroptosis pathway. In some embodiments, the nucleic acid inhibitor downregulates ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS. In specific, non-limiting examples, the nucleic acid inhibitor downregulates ACSL4, LPCAT3, NCOA4, or ATG5. In another specific, non-limiting example, the nucleic acid inhibitor downregulates ACSL4. In certain embodiments, the nucleic acid inhibitor of a component of the ferroptosis pathway is a siRNA, a ribozyme, or a shRNA. In certain embodiments, the donor liver is administered, in an ex vivo perfusion system, a effective amount of a viral vector comprising the nucleic acid inhibitor of a component of the ferroptosis pathway, such as a viral vector comprising a nucleic acid molecule encoding ACSL4, LPCAT3, NCOA4, or ATG5. In a specific, non-limiting example, the nucleic acid inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS is administered to the donor liver using lipid nanoparticles.

In still other embodiments, a donor liver is administered, in an ex vivo perfusion system, a therapeutically effective amount of a nucleic acid molecule encoding an enzyme that increases glutathione production. In some embodiments, the enzyme is GLS2, SLC3A2, SLC7A11, SLC38A1, SLC1A5, or GSR. In certain embodiments, the nucleic acid molecule encoding an enzyme that increases glutathione production is an mRNA. In certain embodiments, the donor liver is administered, in an ex vivo perfusion system, an effective amount of a viral vector comprising the nucleic acid molecule encoding an enzyme that increases glutathione production, such as a viral vector comprising a nucleic acid molecule encoding GLS2, SLC3A2, SLC7A11, SLC38A1, SLC1A5, or GSR. In a specific, non-limiting example, the nucleic acid molecule encoding GLS2, SLC3A2, SLC7A11, SLC38A1, SLC1A5, or GSR is administered to the donor liver using lipid nanoparticles.

Hypothermic machine perfusion provides dynamic organ preservation at 4° C. with protracted infusion of metabolic substrates to the graft during the ex vivo period. It has been used instead of static cold storage (SCS) or after SCS as short perfusion in the transplant center (Tchilikidi. World J Gastrointest Surg. 11(3):126-142, 2019). Based on perfusion technique, such as single- versus double-vessel perfusion and whether active oxygenation of the perfusate is performed, hypothermic machine perfusion can be categorized as follows: (1) HMP: hypothermic machine perfusion; (2) HOPE: hypothermic oxygenated machine perfusion; and (3) D-HOPE: dual hypothermic oxygenated machine perfusion. In clinically used HMP and D-HOPE, liver grafts are perfused throughout the portal vein and the hepatic artery simultaneously, classifying them as double-vessel systems. A single vessel approach is followed in HOPE, with organ perfusion exclusively via the portal vein. While in HOPE and D-HOPE active oxygenation of the perfusate is performed, it is omitted in HMP (Michelotto et al. Langenbecks Arch Surg. 406(1): 39-54, 2021).

Subnormothermic machine perfusion (SNMP) systems have been investigated to assume an intermediate role, benefiting from a lower metabolic demand at subphysiological temperature (e.g., 21° C.), while still maintaining sufficient metabolism for viability testing and improvement of graft function. Once connected to the perfusion system, a donor liver is warmed up gradually, for example reaching ambient room air temperature of 20.8±1.0° C. after approximately 60 minutes.

Based on ongoing research and technological improvements, normothermic (liver) machine perfusion (NMP or normothermic machine liver perfusion) appears to be gaining more traction as compared to subnormothermic perfusion and hypothermic machine perfusion (HMP). NMP of the liver preserves the graft at near-physiological condition. The liver is quickly prepared by removing excess tissue and cannulating the blood vessels while the liver is in static cold storage (SCS); it is then connected to a heparinized circuit filled with warm, oxygenated blood and supplied with nutrients. Devices currently in clinical use for NMP or SNMP procedures include the Organ Assist's Liver Assist (with an adjustable temperature range from 10°−38° C.; for performance of hypothermic and normothermic perfusion), the OrganOx Metra (normothermic perfusion) and the Transmedics OCS™ Liver Portable Perfusion System (normothermic perfusion). Overall, these systems consist of a hepatic artery+/−portal vein pump, a perfusate reservoir and an oxygenating chamber of oxygenated perfusion. These machines vary in portability, degree of automation, perfusate recirculation technique, and the way hepatic arterial flow is delivered. The Metra device (OrganOx Ltd., Oxford, United Kingdom) is a fully automated portable device, perfusing the liver at 37° C. via a cannulated, closed perfusion circuit that provides continuous (nonpulsatile) arterial flow. The Organ Care System (OCS) Liver produced by TransMedics (Andover, MA) is a fully automated and portable device, perfusing livers at normothermic temperature. It delivers a pulsatile flow through the hepatic artery and recirculates perfusate via a reservoir into which the hepatic venous outflow empties. The Liver Assist (Organ Assist, Groningen, the Netherlands) is semiautomated with limited portability. It does allow liver perfusion at temperatures ranging from 8° C. to 37° C., and the arterial and portal pressures can be set by the operator to control the vascular flow rates. It delivers pulsatile flow to the hepatic artery recirculates via an open reservoir. With its limited portability, the device is primarily designed for end-ischemic reconditioning (Mergental and Roll, Clin Liver Dis (Hoboken). 10(4): 97-99, 2017).

Liver perfusates are known to those of ordinary skill in the art. For example, Gelofusine, Steen, and packed red blood cells (PRBCs) plus plasma are solutions that have been used in NMP systems. Gelofusine is a solution made of bovine-derived gelatin, which contains succinylated gelatin as the source of oncotic pressure. Gelofusine is used in Europe as a plasma expander in trauma and shock situations. Gelofusine provides physiologic concentration of Na⁺ and Cl⁻, with low concentrations of K⁺ and Ca⁺⁺. It has an important effect on increasing intravascular volume and oxygen delivery but also at decreasing clot formation. Steen solution has physiologic levels of Na⁺, Cl⁻, and K⁺. It also contains human albumin and dextran (D)-40, both characterized for expanding the intravascular volume. These agents decrease the oxidative response by scavenger effect on hydroxyl, superoxide, hydrogen peroxide, and peroxynitrite. The solution has also shown a decrease in the excessive platelet/leucocyte interaction with the endothelium. Steen as perfusate had previously been tested in pig liver transplant studies and has been extensively used in normothermic ex vivo lung perfusion. Gelofusine in contrast has been used for the majority of NMP in European clinical studies. In addition, PRBCs plus fresh frozen plasma (FFP) have been used as perfusate in a single-center clinical trial with a homemade NMP machine (Linares-Cervantes et al. Transplant Direct. 5(4):e437, 2019).

### GSH and Methods of GSH Administration to Promote Survival of Donor Liver Cells

It is disclosed herein that GSH and/or a supplement that increases GSH in the subject, such as Glutamine, Glutamate, GSSG, Cysteine, Glycine, Cys-Gly, Choline, Phosphatidylcholine, Sorbitol, Palmitoylcarnitine, Methylthioadenosine, S-Adenosylhomocysteine, S-Adenosylmethionine, Cystine, Methionine Creatine, Gamma-aminobutyrate, Guanadinoacetate, a pharmaceutically acceptable salt thereof, or any combination thereof, can be used to increase survival of a donor liver in a liver transplant recipient, wherein the donor liver is homozygous for rs738409:G mutation. In one non-limiting example, GSH is administered to the subject. In some embodiments, administering GSH to the subject inhibits lipid peroxidation and/or mitochondrial shrinkage in donor liver cells, and/or increases survival of donor liver cells in the subject.

Pharmaceutical compositions that include GSH and/or a supplement that increases glutathione in the subject, wherein the supplement is Glutamine, Glutamate, GSSG, Cysteine, Glycine, Cys-Gly, Choline, Phosphatidylcholine, Sorbitol, Palmitoylcarnitine, Methylthioadenosine, S-Adenosylhomocysteine, S-Adenosylmethionine, Cystine, Methionine Creatine, Gamma-aminobutyrate, Guanadinoacetate, a pharmaceutically acceptable salt thereof, or any combination thereof, can be formulated with an appropriate pharmaceutically acceptable carrier and used in the methods disclosed herein. The dosage form of the pharmaceutical composition will be determined by the mode of administration chosen. Exemplary administration methods include, but are not limited to, topical, oral, subcutaneous, transdermal, intrathecal, intramuscular, intravenous, intraperitoneal, and similar administration routes, or combinations thereof. For instance, in addition to injectable fluids, topical, inhalation, oral, infusion, and suppository formulations can be employed. Oral formulations can be liquid (such as syrups, solutions, or suspensions) or solid (such as powders, pills, tablets, or capsules). Suppository preparations can also be solid, gel, or in a suspension form. Infusion preparations, administered by catheter, are generally administered as liquids. Inhalation preparations can be liquid (such as solutions or suspensions) and include mists, sprays, and the like. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art. In some embodiments, the pharmaceutical composition can be formulated for oral, intramuscular, or intravenous administration.

The amount of GSH or a supplement that increases glutathione in the subject, such as Glutamine, Glutamate, GSSG, Cysteine, Glycine, Cys-Gly, Choline, Phosphatidylcholine, Sorbitol, Palmitoylcarnitine, Methylthioadenosine, S-Adenosylhomocysteine, S-Adenosylmethionine, Cystine, Methionine Creatine, Gamma-aminobutyrate, Guanadinoacetate, a pharmaceutically acceptable salt thereof, or any combination, that is administered, will be dependent on the subject being treated, the severity of the affliction, and the manner of administration and is best left to the judgment of the prescribing clinician. Within these bounds, the formulation to be administered will contain a quantity of the active ingredient(s) in amounts effective to achieve the desired effect in the subject being treated. A therapeutically effective amount can be the amount that is necessary to treat or lower the risk of a subject for decreased liver function of a transplant, or increase the size of the transplant. In some embodiments, the administration results in improved survival of the transplanted liver.

The pharmaceutical compositions that include GSH and/or a supplement that increases glutathione in the subject, wherein the supplement is at least one of Glutamine, Glutamate, GSSG, Cysteine, Glycine, Cys-Gly, Choline, Phosphatidylcholine, Sorbitol, Palmitoylcarnitine, Methylthioadenosine, S-Adenosylhomocysteine, S-Adenosylmethionine, Cystine, Methionine Creatine, Gamma-aminobutyrate, or Guanadinoacetate, a pharmaceutically acceptable salt thereof, or any combination, can be formulated in unit dosage form, suitable for individual administration of precise dosages. A variety of dosages and dosing regimens are possible (for example, Kilpatrick et al., International Immunopharmacology, 3:541-548, 2003; Gandhi et al., Blood, 106(13):4253-4260, 2005, both of which are incorporated herein by reference in their entireties). In one specific, non-limiting example, a unit dosage (such as intravenous dosage or an oral dosage) can be used.

In some embodiments, the subject is administered (for example, orally) about 50 to about 1500 mg of GSH, such as about 50 mg to about 150 mg, about 150 mg to about 300 mg, about 300 mg to about 450 mg, about 450 mg to about 600 mg, about 600 mg to about 750 mg, about 750 mg to about 900 mg, about 900 mg to about 1150 mg, about 1150 mg to about 1300 mg, or about 1300 mg to about 1500 mg. In one specific non-limiting example, the subject is administered (for example, orally) about 300 mg of GSH per day. In another specific, non-limiting example, the subject is administered (for example, intravenously) about 1400 mg in a single dose or in more than dose.

The dose administered to a subject should be sufficient to induce a beneficial therapeutic response in a subject over time, or to promote survival of a donor liver in a recipient subject. The beneficial therapeutic response may require one or more doses, such as 1, 2, 3, 4, 5, 6, 7, 8, 9, 10, 20, 30, 40, 50, 100, 200, 300, 400, or 500, or more doses, administered at the same or different times. The dose may vary from subject to subject or may be the same. An appropriate dose can be determined by one of ordinary skill in the art using routine experimentation.

Pharmaceutically acceptable salts and hydrates are also of use in the disclosed methods. In one embodiment, and pharmaceutically acceptable salt is a chloride salt. However, other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

A variety of administration regimens are possible (for example, Kilpatrick et al., International Immunopharmacology, 3:541-548, 2003; Gandhi et al., Blood, 106(13):4253-4260, 2005, both of which are incorporated herein by reference in their entireties). Administration with a therapeutically effective amount can be a single administration or multiple administrations. Administration can involve daily or multi-daily or less than daily (such as weekly, monthly, etc.) doses over a period of a few days to weeks or months, or even years. In a particular non-limiting example, administration involves once daily dose or twice daily dose. The particular mode/manner of administration and the dosage regimen will be selected by the attending clinician, taking into account the particulars of the case (such as the subject, the disease, the disease state/severity involved, the particular administration, and whether the treatment is prophylactic). In specific, non-limiting examples, administration can be oral, intramuscular, or by intravenous delivery.

In some embodiments, for the use of GSH, and/or a supplement that increases glutathione in the subject, the composition is administered daily. In other embodiments the composition is administered more than once a day, such as twice a day, three time a day or four times a day. In yet other embodiments, the composition is administered once a day, every other day, every three days or once a week. In some embodiments, the GSH or a supplement that increases glutathione in the subject is administered by an intravenous (IV) infusion, such as within one day after transplantation or resection, and continued for at least 7 days, such as 8, 9, 10, 11, 12, 13 or 14 days, such as for about 7 to about 14 days. However, the IV infusion can be continued for longer periods, such as for up to three weeks. In a liver transplant donor, and IV infusion can be administered before and/or after a resection procedure. In some embodiments, GSH or a supplement that increases glutathione in the subject is administered intravenously, such as using an infusion.

The dosage form of the pharmaceutical composition will be determined by the mode of administration chosen, which can be systemic or localized (such as to the afflicted area). The pharmaceutically acceptable carriers and excipients useful in this disclosure are conventional (see, for example, Remington: The Science and Practice of Pharmacy, The University of the Sciences in Philadelphia, Editor, Lippincott, Williams, & Wilkins, Philadelphia, PA, 21st Edition (2005)). For instance, parenteral formulations usually comprise injectable fluids that are pharmaceutically and physiologically acceptable fluid vehicles, such as water, physiological saline, other balanced salt solutions, aqueous dextrose, glycerol, or the like. In addition to injectable fluids, inhalational, and oral formulations can be employed. For solid compositions (such as powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example, sodium acetate or sorbitan monolaurate. Excipients that can be included are, for instance, proteins, such as human serum albumin or plasma preparations. Actual methods of preparing such dosage forms are known, or will be apparent, to those skilled in the art. The compositions of this disclosure that include GSH and/or a supplement that increases glutathione in the subject can be administered to humans or other animals by any method.

In some examples, the compositions disclosed herein can be administered systemically, such as orally or parenterally, for example, intravenously, intramuscularly, intraperitoneally (i.p.), intranasally, intradermally, intrathecally, subcutaneously, via catheter, via inhalation, or via suppository. In one non-limiting example, the composition is administered orally. For oral administration, the pharmaceutical compositions can take the form of, for example, tablets or capsules prepared by conventional means with pharmaceutically acceptable excipients, such as binding agents (for example, pregelatinized maize starch, polyvinylpyrrolidone, or hydroxypropyl methylcellulose); fillers (for example, lactose, microcrystalline cellulose, or calcium hydrogen phosphate); lubricants (for example, magnesium stearate, talc, or silica); disintegrants (for example, potato starch or sodium starch glycolate); or wetting agents (for example, sodium lauryl sulfate). The tablets can be coated by methods well known in the art. Solid dosage forms for oral administration include, but are not limited to, capsules, tablets, pills, powders, and granules. In such solid dosage forms, the active compounds are mixed with at least one pharmaceutically acceptable excipient or carrier such as, but not limited to, sodium citrate or dicalcium phosphate. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives such as suspending agents (such as sorbitol syrup, cellulose derivatives, or hydrogenated edible fats); emulsifying agents (such as lecithin or acacia); non-aqueous vehicles (such as almond oil, oily esters, ethyl alcohol, or fractionated vegetable oils); and preservatives (such as methyl or propyl-p-hydroxybenzoates or sorbic acid). The preparations can also contain buffer salts, flavoring, coloring, and sweetening agents as appropriate. For solid compositions, conventional non-toxic solid carriers can include pharmaceutical grades of mannitol, lactose, starch, or magnesium stearate. Actual methods of preparing such dosage forms are known, or will be apparent, to those of ordinary skill in the art. Oral administration includes buccal or “sub-lingual” administration via membranes of the mouth. This can be accomplished using lozenges or a chewable gum.

Pharmaceutical compositions suitable for oral administration can be presented in discrete units each containing a predetermined amount of at least one therapeutic compound useful in the present methods; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product.

For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent, or surface active/dispersing agent(s). Molded tablets can be made by molding, in a suitable machine, the powdered compound moistened with an inert liquid diluent.

Solid compositions of a similar type can also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include, but are not limited to, pharmaceutically acceptable emulsions, solutions, suspensions, syrups, teas, and elixirs. In addition to the active compounds, the liquid dosage forms can contain inert diluents commonly used in the art such as, for example, water or other solvents, solubilizing agents, and emulsifiers, such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In some embodiments, suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

A drinkable tea can also be used in the present methods. A drinkable tea may be taken in a liquid form or in a once pulverized or granulated form together with water or hot water. When it is in a powdery or granular form, the drinkable tea may be contained in a cavity of mouth before taking hot water or water like the conventional powdery or granular drinkable tea, or it may be taken after once dissolving in hot water or water. One or more inactive ingredients, such as a sugar, mint, or other flavor, can be added to improve taste and easiness as a drinkable drug. Teas, syrups, and elixirs can be formulated with sweetening agents, for example glycerol, sorbitol, or sucrose. Such compositions can also contain a demulcent, a preservative, and flavoring and coloring agents.

Administration can be intravenous. Optionally, the pharmaceutical composition includes a parenteral carrier, and, in some embodiments, it is a solution that is isotonic with the blood of the recipient. Examples of such carrier vehicles include water, saline, Ringer's solution, and dextrose solution. Non-aqueous vehicles, such as fixed oils and ethyl oleate, are also useful herein, as well as liposomes.

The pharmaceutical compositions may be in the form of particles comprising a biodegradable polymer or a polysaccharide jellifying or bioadhesive polymer, an amphiphilic polymer, an agent modifying the interface properties of the particles and a pharmacologically active substance. These compositions exhibit certain biocompatibility features which allow a controlled release of the active substance. (See U.S. Pat. No. 5,700,486, incorporated herein by reference in its entirety).

In some embodiments, GSH or a supplement that increases glutathione in the subject, wherein the supplement is at least one of Glutamine, Glutamate, GSSG, Cysteine, Glycine, Cys-Gly, Choline, Phosphatidylcholine, Sorbitol, Palmitoylcarnitine, Methylthioadenosine, S-Adenosylhomocysteine, S-Adenosylmethionine, Cystine, Methionine Creatine, Gamma-aminobutyrate, or Guanadinoacetate is included in a controlled release formulation, for example, a microencapsulated formulation. Various types of biodegradable and biocompatible polymers can be used, and methods of encapsulating a variety of synthetic compounds, proteins, and nucleic acids, have been well described in the art (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2nd ed., CRC Press, 2006, all of which are incorporated by reference herein in their entireties).

In other embodiments, GSH and/or a supplement that increases glutathione in the subject, is included in a nanodispersion system. Nanodispersion systems and methods for producing such nanodispersions are well-known to one of skill in the art. (See, for example, U.S. Pat. No. 6,780,324; U.S. Pat. Publication No. 2009/0175953, both of which are incorporated herein by reference in their entireties). For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and PNPase inhibitor or PNPase purine nucleoside substrate (such as 80/20 w/w). In some examples, the nanodispersion is prepared using the solvent evaporation method (see, for example, Kanaze et al., Drug Dev. Indus. Pharm. 36:292-301, 2010; Kanaze et al., J. Appl. Polymer Sci. 102:460-471, 2006, both of which are incorporated herein by reference in their entireties).

Many types of release delivery systems are available and known to those of ordinary skill in the art. They include polymer-based systems, such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109 (incorporated herein by reference in its entirety). Delivery systems also include non-polymer systems, such as lipids, including sterols such as cholesterol, cholesterol esters and fatty acids or neutral fats, such as mono-, di-, and tri-glycerides; hydrogel release systems; silastic systems; peptide-based systems; wax coatings; compressed tablets using conventional binders and excipients; partially fused implants; and the like. Specific examples include, but are not limited to, (a) erosional systems in which a PNPase inhibitor or a PNPase purine nucleoside substrate is contained in a form within a matrix, such as those described in U.S. Pat. Nos. 4,452,775; 4,667,014; 4,748,034; 5,239,660; and 6,218,371 (all of which are incorporated by reference herein in their entireties) and (b) diffusional systems in which an active ingredient permeates at a controlled rate from a polymer, such as described in U.S. Pat. Nos. 3,832,253 and 3,854,480 (both of which are incorporated by reference in their entireties). In addition, pump-based hardware delivery systems can be used, some of which are adapted for implantation.

### Methods of Preventing or Reducing Programmed Cell Death in a Donor Liver Carrying the PNPLA3 rs738409:G Variant

Provided herein are methods of promoting survival of a donor liver in a recipient subject, wherein the donor liver is homozygous for rs738409:G mutation in the PNPLA3 gene. In some embodiments, the therapies disclosed herein are administered to the transplant recipient subject. In some embodiments, the therapies disclosed herein are administered to the donor liver prior to transplantation, such as in an ex vivo perfusion system. In some embodiments, the therapies disclosed herein are administered to a living donor prior to liver transplantation to the recipient. In embodiments herein, the disclosed therapeutics can be administered to the transplant recipient subject, the donor liver (such as in an ex vivo perfusion system), the living donor prior to liver transplantation, or combinations thereof. The disclosed methods increase survival of donor hepatocytes, as compared to survival of the donor hepatocytes in the absence of treatment. In further embodiments, the disclosed methods increase proliferation of donor hepatocytes, as compared to proliferation of the donor hepatocytes in the absence of treatment. In some embodiments, the disclosed methods inhibit lipid peroxidation and/or mitochondrial shrinkage in donor liver cells, and/or increase survival of donor liver cells in the subject.

In some embodiments, the method includes detecting the rs738409:G mutation in the PNPLA3 gene in a sample from the donor. In some embodiments, the sample from the donor is a blood or tissue sample. In specific, non-limiting embodiments, the sample from the donor is a liver tissue sample. A variety of techniques for detecting a mutation in a gene, such as a rs738409:G mutation in the PNPLA3 gene, are known to those of ordinary skill in the art, and can be used in the disclosed methods. Such methods can include, for example, polymerase chain reaction (PCR). PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989).

In some embodiments, the subject can have a partial liver resection. In some embodiments, the subject can be a recipient of a liver transplant, such as a cadaveric transplant or a transplant from a living donor. The subject can be a mammal, such as a domestic animal or a primate. In some examples, the subject is a human.

In some embodiments, the individual has undergone a partial hepatectomy or liver resection. In some non-limiting examples, the partial hepatectomy or liver resection removed 5%, 10%, 15%, 20%, 25%, 30%, 35%, 40%, 45%, 50%, 55%, 60%, 65%, 70%, 75%, 80%, 85%, 90%, or 95% by mass of the subject's liver.

In some embodiments, the subject is the recipient of a liver transplant from a liver donor. In some embodiments, the hepatectomy is anatomic, so that the lines of resection match the limits of one or more functional segments of the liver as defined by the Couinaud classification. The subject can be an adult (over 18 years old), or a child (under 13 years old) or a teenager (13 to 19 years old). The subject can be over 20, 30, 40, 50, or 60 years old.

In further embodiments, a subject is treated using the method disclosed herein that has undergone a liver transplant, and is a transplant recipient. In further embodiments the subject has undergone a small-for-size liver transplant. In some embodiments, the subject has undergone a liver transplant due to liver damage caused by toxic injury, traumatic injury, microvesicular steatosis, or macrovesicular steatosis. In some non-limiting examples, the toxic injury results from acetominophen overdose, exposure to carbon tetrachloride (CCl₄), bacterial endotoxin, use or abuse of intravenous or prescription drugs, chemotherapy, excessive consumption of alcohol, or infection with hepatitis virus A, B, or C. Traumatic injury can result from surgical resection or blunt force trauma, such as that occurring in an automobile accident. In some embodiments, the subject has received an extended criteria liver, such as, but not limited to, a liver harvested from a subject that is greater than about 45 years old, such as about 45 to about 55 years old, such as about 45 to old 50 years old. In further embodiments, the subject has received a cadaveric liver. In yet other embodiments, the subject has received a liver transplant from a living donor.

In certain embodiments, the method of promoting survival of a donor liver in a subject in need thereof comprises administering to the subject a pharmaceutical composition as disclosed herein. In specific non-limiting embodiments, the pharmaceutical compositions includes GSH or a nucleic acid molecule encoding GPX4. The dose and dosing schedule for administration of GSH or a nucleic acid molecule encoding GPX4 can vary and is determined in part by the clinical status of the subject or the donor liver, and the age, such as the weight and general health of the subject, and the route of administration. Suitable formulations are disclosed above, as well as dosing regimens.

In some embodiments, the pharmaceutical composition is administered to the donor liver. The pharmaceutical composition can be administered to the donor liver within 24 hours of transplantation, such as within 12 hours of transplantation, such as within 6 hours of transplantation, such as about 6, 5, 4, 3, 2 or 1 hour prior to transplantation. Suitable methods and formulations are disclosed above.

In further embodiments, the GSH, the nucleic acid molecule encoding GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, or the nucleic acid inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS can be administered within one day of a surgical procedure, such as a liver resection or a liver transplantation, for example in a liver donor or in a liver recipient. The GSH, the nucleic acid molecule encoding GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, or the nucleic acid inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS can be administered within 1, 2, 3, 4, 5, 6, 7, 8, 9, 10, 11, 12, 24, 36, 48, 72 or 96 hours of the surgical procedure, and continued, as disclosed above. The GSH, the nucleic acid molecule encoding GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, or the nucleic acid inhibitor of ACSL4, NCOA4, ATG5, LPCAT3, ZEB1, ST3GAL5, SLC33A1, ST3GAL2, ST6GALNAC4, HS3ST3A1, B4GALNT1, ST6GALNAC6, HEXB, HYAL4, or GNS can be immediately administered after a procedure, such as, but not limited to, in a liver transplant recipient or liver transplant donor. The GSH, the nucleic acid molecule encoding GPX4, GPX1, GPX2, GPX3, FSP1, SCD, FADS2, GLS2, NFE2L2, BACH1, CREB1, GRSF1, TFAP2C, HMGCR, GCK, CYP1A1, SLC3A2, SLC7A11, SLC38A1, SLC1A5, or GSR protein, or the nucleic acid inhibitor of ACSL4, NCOA4, ATG5, LPCAT3, ZEB1ST3GAL5, SLC33A1, ST3GAL2, ST6GALNAC4, HS3ST3A1, B4GALNT1, ST6GALNAC6, HEXB, HYAL4, or GNS can be administered before a procedure, such as, but not limited to, to a liver transplant donor or recipient, or into the liver that is to be transplanted.

The subject can be administered additional therapeutic agents. Additional agents that can be administered to the subject include immunosuppressive therapeutics, antibacterial and antifungal antibiotics, as well as non-steroidal anti-inflammatory agents to reduce risk of infection and inflammation. Additional agents of use include a therapeutically effective amount of deferoxamine, selenium, vitamin E (alpha-tocopherol), CoQ10, or a combination thereof. Additional agents can be administered by any route. The additional agents can be formulated separately, or in the same composition.

Additional agents of use may also include Acyclovir (ZOVIRAX®), Ganciclovir (CYTOVENE®), Azathioprine (IMURAN®), Cyclophosphamide (CYTOXAN®), Cyclosporine (SANDIMMUNE®, NEORAL®), Muromonab-CD3 (ORTHOCLONE OKT3®), Mycophenolate Mofetil (CELLCEPT®), Prednisone (DELTASONE®), Sirolimus (RAPAMYCIN®, RAPAMUNE®), Tacrolimus (PROFRAG®), Ferrous Sulfate (FEOSOL®, SLOW FE®), Magnesium Oxide (MAG-OX®, Magnesium Gluconate (MAGONATE®), Dapsone (AVLOSULFON®), Docusate (COLACE®), Fludrocortisone (FLORINEF®), Nifedipine (PROCARDIA XL®, ADALAT CC®), Nystatin (MYCOSTATIN®, NILSTAT®), Pentamidine Isethionate Nebulization Solution (NEBUPENT®, PENTAM®), Sodium Bicarbonate, Sulfamethoxazole with Trimethoprim (BACTRIM®, SEPTRA®), or combinations thereof. Agents of use can include antibiotics such as minoglycosides (for example, amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (for example, azidamfenicol, chloramphenicol, florfenicol, thiamphenicol), ansamycins (for example, rifamide, rifampin, rifamycin sv, rifapentine, rifaximin), β-lactams (for example, carbacephems (e.g., loracarbef), carbapenems (for example, biapenem, imipenem, meropenem, panipenem), cephalosporins (for example, cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (for example, cefbuperazone, cefmetazole, cefininox, cefotetan, cefoxitin), monobactams (for example, aztreonam, carumonam, tigemonam), oxacephems, flomoxef, moxalactam), penicillins (for example, amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin g benzathine, penicillin g benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), other (for example, ritipenem), lincosamides (for example, clindamycin, lincomycin), macrolides (for example, azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (for example, amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (for example, apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin). Agents of use also include synthetic antibacterials, such as 2,4-Diaminopyrimidines (for example, brodimoprim, tetroxoprim, trimethoprim), nitrofurans (for example, furaltadone, furazolium chloride, nifuradene, nifuratel, nifurfoline, nifurpirinol, nifurprazine, nifurtoinol, nitrofurantoin), quinolones and analogs (for example, cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (for example, acetyl sulfamethoxypyrazine, benzylsulfamide, chloramine-b, chloramine-t, dichloramine t, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (for example, acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (for example, clofoctol, hexedine, methenamine, methenamine anhydromethylene-citrate, methenamine hippurate, methenamine mandelate, methenamine sulfosalicylate, nitroxoline, taurolidine, xibornol).

Additional agents of use include antifungal antibiotics such as polyenes (for example, amphotericin B, candicidin, dennostatin, filipin, fungichromin, hachimycin, hamycin, lucensomycin, mepartricin, natamycin, nystatin, pecilocin, perimycin), others (for example, azaserine, griseofulvin, oligomycins, neomycin undecylenate, pyrrolnitrin, siccanin, tubercidin, viridin) allylamines (for example, butenafine, naftifine, terbinafine), imidazoles (for example, bifonazole, butoconazole, chlordantoin, chlormiidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (for example, tolciclate, tolindate, tolnaftate), triazoles (for example, fluconazole, itraconazole, saperconazole, terconazole) others (for example, acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate). Antineoplastic agents can also be of use including (1) antibiotics and analogs (for example, aclacinomycins, actinomycin, anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin), (2) antimetabolites such as folic acid analogs (for example, denopterin, edatrexate, methotrexate, piritrexim, pteropterin, trimetrexate), (3) purine analogs (for example, cladribine, fludarabine, 6-mercaptopurine, thiamiprine, thioguanine), (4) pyrimidine analogs (for example, ancitabine, azacitidine, 6-azauridine, carmofur, cytarabine, doxifluridine, emitefur, enocitabine, floxuridine, fluorouracil, gemcitabine, tagafur).

Steroidal anti-inflammatory agents can also be used such as 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, cyclosporine, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

In addition, non-steroidal anti-inflammatory agents can be used. These include aminoarylcarboxylic acid derivatives (for example, enfenamic acid, etofenamate, flufenamic acid, isonixin, meclofenamic acid, mefenamic acid, niflumic acid, talniflumate, terofenamate, tolfenamic acid), arylacetic acid derivatives (for example, aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (for example, bumadizon, butibufen, fenbufen, xenbucin), arylcarboxylic acids (for example, clidanac, ketorolac, tinoridine), arylpropionic acid derivatives (for example, alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (for example, difenamizole, epirizole), pyrazolones (for example, apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (for example, acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (for example, ampiroxicam, droxicam, isoxicam, lornoxicam, piroxicam, tenoxicam), .epsilon.-acetamidocaproic acid, s-adenosylmethionine, 3-amino-4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

The disclosed methods can include measuring liver function and/or survival using a quantitative and/or qualitative test. In some embodiments, the degree of liver impairment is assessed using tests which evaluate structure (e.g., biopsy), cellular permeability (e.g., transaminases) and synthetic ability (e.g., albumin, bilirubin and prothrombin time) (see Jalan and Hayes (1995) Aliment. Pharmacol. Ther. 9:263-270). A combination of various markers for liver injury can be measured to provide an analysis function. Commonly used tests for liver clearance capability are: indocyanine green (ICG), galactose elimination capacity (GEC), mono-ethyl-glycine-xylidide (MEG-X), antipryine clearance, aminopyrine breath test (ABT) and caffeine clearance. For assessment of graft function following transplantation, low ICG clearance and low MEG-X formation are predictive of a poor outcome. The method can also include measuring the lipid profile of a subject. The method can include measuring liver size, such as using ultrasound.

### EXAMPLES

The following examples are provided to illustrate particular features of certain embodiments, but the scope of the claims should not be limited to those features exemplified.

### Example 1

### Material and Methods

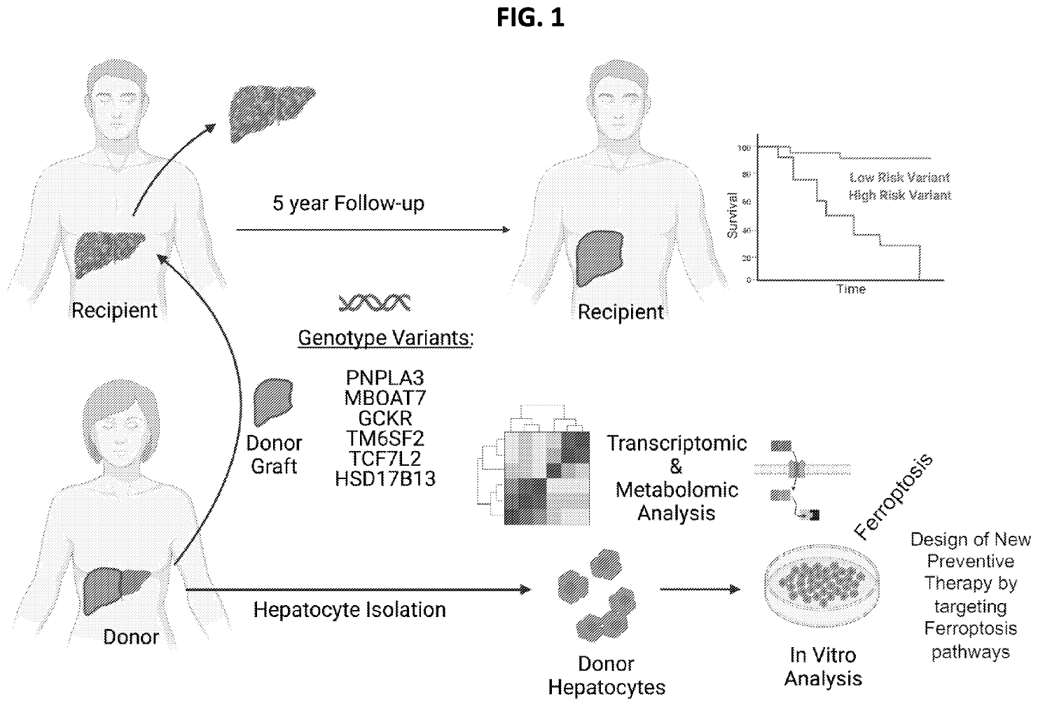

Study population: Eighty-three recipients and 79 donors who underwent living donor liver transplantation at the Department of Surgery and Science, Kyushu University Hospital in Japan, between 2004 and 2018 were included in the present retrospective cohort study. Recipients with a positive history of viral hepatitis were excluded from this part of the study. Human hepatocytes were also procured. Information regarding age, gender, and cell viability of human liver tissue and hepatocytes used in this study is described in Table 1. FIG. 1 shows a schematic of the living donor liver transplantation process described herein.

TABLE 1
Healthy human hepatocytes used in this study.

| Hepatocyte | Age | | | PNPLA3 | MBOAT | GCKR | HSD17B13 |
|---|---|---|---|---|---|---|---|
| ID | (Years) | Sex | Viability | rs738409 | rs641738 | rs780094 | rs72613567 |
| 1 | 77 | female | 85% | GG | CC | CC | TT |
| 2 | 11 | male | 96% | CG | CT | TT | TT |
| 3 | 15 | male | 95% | CG | TT | CC | TT |
| 4 | 46 | male | 80% | GG | CC | CT | TT |
| 5 | 53 | male | 86% | GG | CC | CC | TT |
| 6 | 54 | male | 80% | GG | CC | CC | TT |
| 7 | 50 | male | 97% | GG | CC | CT | TT |
| 8 | 45 | male | 92% | CC | CT | CT | TT |
| 9 | 49 | female | 86% | CC | CT | CT | TTA |
| 10 | 31 | female | 91% | CG | CC | CC | TT |
| 11 | 40 | female | 89% | GG | TT | CT | TTA |
| 12 | 74 | male | 86% | CG | CT | TT | TT |
| 13 | 33 | female | 84% | CG | CT | CC | TT |
| 14 | 74 | female | 90% | CC | CT | CC | TTA |
| 15 | 32 | male | 84% | CC | CC | CC | TTA |
| 16 | 47 | male | 83% | GG | CC | CT | TT |
| 17 | 29 | male | 88% | CC | CC | CC | TT |
| 18 | 16 | male | 82% | CC | CT | CC | TTA |
| 19 | 58 | female | 91% | CC | CT | CC | TT |
| 20 | 37 | female | 84% | CC | CT | CC | TT |
| 21 | 31 | female | 90% | CG | CC | CT | TT |
| 22 | 36 | male | 94% | CG | TT | TT | TT |
| 23 | 44 | male | 87% | CG | CC | CC | TTA |
| 24 | 28 | female | 92% | CC | CT | — | — |
| 25 | 55 | male | 90% | CG | TT | CC | TT |
| 26 | 47 | female | 81% | CG | CT | CC | TT |
| 27 | 61 | male | 88% | CG | CT | CC | TTA |
| 28 | 40 | male | 89% | CC | TT | CC | TT |
| 29 | 48 | female | 82% | CC | CC | CC | TTA |
| 30 | 74 | male | 90% | CC | CC | TT | TTA |
| 31 | 48 | female | 91% | CG | CC | TT | TTA |
| 32 | 58 | male | 81% | CC | TT | CC | TT |
| 33 | 52 | female | 90% | CC | CT | CT | TTA |
| 34 | 40 | female | 92% | CG | CT | CC | TT |
| 35 | 34 | male | 84% | CC | CT | CT | TT |
| 36 | 35 | male | 84% | CC | CT | CT | TTA |
| 37 | 9 | male | 80% | CC | TT | TT | TT |
| 38 | 4 | female | 91% | CG | CC | CC | TT |
| 39 | 56 | male | 84% | CC | TT | CT | TT |
| 40 | 44 | female | 85% | CC | CT | CC | TT |
| 41 | 16 | male | 80% | CC | CT | CC | TT |
| 42 | 38 | male | 90% | CC | CT | CT | TT |
| 43 | 61 | female | 90% | CG | CT | CT | TT |
| 44 | 57 | male | 92% | CC | CT | CC | TT |
| 45 | 44 | male | 89% | CC | CC | TT | TT |
| 46 | 23 | male | 95% | CC | CC | CT | TTA |
| 47 | 64 | female | 87% | CG | CC | CT | TTA |
| 48 | 33 | female | 84% | CG | TT | TT | TT |
| 49 | 15 | male | 81% | CG | CT | CC | TT |
| 50 | 41 | male | 93% | CC | CT | CC | TT |
| 51 | 0 | male | 90% | CC | CT | CC | TTA |
| 52 | 4 | female | 92% | CC | CT | CT | TT |
| 53 | 5 | male | 96% | CG | CC | CT | TT |
| 54 | 48 | Female | 83% | CC | CT | CC | TT |
| 55 | 45 | Female | 95% | CG | TT | CT | TT |
| 56 | 48 | Female | 84% | CC | CT | CC | TT |
| 57 | 43 | Female | 69% | GG | CC | TT | TT |
| 58 | 46 | Male | 88% | CG | CT | CT | TT |
| 59 | 44 | Male | 85% | CG | CC | CT | TT |
| 60 | 66 | Male | 84% | CG | TT | CC | TTA |
| 61 | 71 | Female | 86% | CC | CT | CT | TT |
| 62 | 26 | Female | 96% | CG | CC | TT | TTA |
| 63 | 52 | Male | 82% | CC | CC | CC | TT |
| 64 | 53 | Female | 95% | CC | CT | CT | TT |
| 65 | 28 | Male | 81% | CG | CT | CC | TT |
| 66 | 42 | Male | 95% | CG | CT | CT | TTA |
| 67 | 39 | Female | 89% | CC | CC | CC | TT |
| 68 | 51 | Male | 81% | CC | CT | CC | TT |
| 69 | 71 | Female | 94% | CC | CC | CC | TT |
| 70 | 62 | Male | 92% | CC | CC | CT | TT |
| 71 | 21 | Female | 84% | CG | TT | CC | TTA |
| 72 | 59 | Female | 92% | CC | CC | TT | TTA |
| 73 | 42 | Male | 92% | CC | CC | TT | TTA |
| 74 | 68 | Female | 95% | CC | CC | CC | TTA |
| 75 | 55 | Male | 92% | CC | CC | CC | TT |
| 76 | 37 | Male | 90% | GG | CT | TT | TT |
| 77 | 45 | Male | 93% | CG | CT | CT | TTA |
| 78 | 57 | Female | 95% | CC | CT | CT | TTA |
| 79 | 58 | Female | 89% | CC | CT | CT | TATA |
| 80 | 37 | Female | 80% | CC | CT | CT | TT |
| 81 | 33 | Female | 81% | CC | CT | CC | TT |
| 82 | 77 | Female | 92% | CG | CC | CC | TT |
| 83 | 44 | Female | 95% | CG | CC | CC | TT |
| 84 | 24 | Female | 91% | CG | CT | CT | TT |
| 85 | 69 | Female | 87% | CG | TT | CT | TT |
| 86 | 35 | Male | 84% | CC | CT | CC | TT |
| 87 | 44 | Male | 83% | CG | CC | CT | TT |
| 88 | 31 | Male | 92% | CG | CC | CT | TT |
| 89 | 46 | Female | 89% | CG | CT | CC | TT |
| 90 | 1.3 | Male | 91% | CC | CC | CT | TTA |
| 91 | 57 | Female | 90% | CG | TT | TT | TT |
| 92 | 27 | Female | 97% | GG | CC | CT | TT |
| 93 | 65 | Female | 90% | CC | CC | CC | TT |
| 94 | 24 | Female | 90% | CG | TT | CT | TT |
| 95 | 45 | Male | 96% | CC | TT | CC | TT |
| 96 | 55 | Female | 86% | CG | TT | CC | TTA |
| 97 | 27 | Female | 93% | CG | CC | CT | TTA |
| 98 | 56 | Female | 95% | CC | CT | CC | TTA |
| 99 | 49 | Female | 81% | CG | CT | CT | TT |
| 100 | 66 | Female | 94% | CC | CT | CC | TT |
| 101 | 64 | Female | 96% | CG | CT | TT | TT |
| 102 | 65 | Male | 83% | CC | CT | CC | TT |
| 103 | 60 | Female | 87% | CG | TT | CT | TT |
| 104 | 47 | Female | 98% | CC | CC | CC | TTA |
| 105 | 22 | Male | 85% | CC | TT | CT | TTA |
| 106 | 15 | Male | 80% | CC | CT | CT | TTA |
| 107 | 64 | Female | 88% | GG | CT | CT | TATA |
| 108 | 17 | Male | 86% | CC | CT | CT | TTA |
| 109 | 51 | Male | 83% | CC | CC | CT | TTA |
| 110 | 49 | Male | 86% | CC | TT | CT | TT |
| 111 | 46 | Female | 86% | GG | TT | TT | TT |
| 112 | 45 | Male | 88% | CC | CC | CT | TT |
| 113 | 69 | Female | 90% | CC | TT | CC | TT |
| 114 | 53 | Male | 87% | CC | CT | CT | TT |
| 115 | 57 | Male | 95% | CC | CT | CT | TT |
| 116 | 67 | Male | 86% | CG | CC | CC | TTA |
| 117 | 44 | Male | 85% | CG | TT | CC | TT |
| 118 | 50 | Male | 93% | CC | TT | CT | TATA |
| 119 | 52 | Female | 90% | CC | CT | CC | TT |
| 120 | 17 | Male | 89% | CG | TT | CC | TT |
| 121 | 18 | Female | 89% | CG | TT | CC | TT |
| 122 | 0 | Female | 84% | CC | CT | CC | TT |
| 123 | 49 | Male | 85% | CC | CC | CT | TT |
| 124 | 55 | Female | 88% | CC | CT | CT | TT |
| 125 | 61 | Female | 92% | CG | CT | CT | TT |
| 126 | 18 | Male | 92% | CG | CC | TT | TTA |
| 127 | 41 | Female | 89% | CC | CC | CC | TTA |
| 128 | 53 | Female | 92% | CG | TT | CC | TT |
| 129 | 66 | Female | 81% | CC | CT | CT | TTA |
| 130 | 33 | Male | 91% | CG | CT | CT | TT |
| 131 | 48 | Male | 80% | CG | CT | CT | TT |
| 132 | 40 | Male | 94% | CC | TT | CC | TTA |
| 133 | 62 | Female | 89% | CG | CT | CT | TTA |
| 134 | 41 | Male | 84% | CC | CC | CT | TT |
| 135 | 57 | Male | 87% | CC | CT | CC | TTA |
| 136 | 19 | Female | 86% | CC | CT | CT | TT |
| 137 | 62 | Female | 87% | CC | CC | TT | TT |
| 138 | 59 | Female | 82% | CG | CT | CC | TTA |
| 139 | 27 | Male | 86% | CC | CT | CC | TTA |
| 140 | 53 | Female | 85% | CC | CC | CT | TT |
| 141 | 52 | Female | 92% | GG | CT | CT | TTA |
| 142 | 25 | Male | 94% | CG | CC | CT | TT |
| 143 | 54 | Male | 84% | CC | CT | CC | TT |
| 144 | 24 | Male | 81% | CG | CT | CC | TTA |
| 145 | 40 | Male | 100% | CG | CT | CT | TTA |
| 146 | 43 | Male | 93% | CC | CT | CC | TTA |
| 147 | 0 | Female | 90% | CG | CC | CT | TTA |
| 148 | 0 | Male | 87% | CC | CC | CT | TATA |
| 149 | 45 | Female | 83% | CG | TT | TT | TTA |
| 150 | 26 | Female | 97% | CC | CC | CT | TTA |
| 151 | 37 | Female | 82% | CC | CT | CT | TT |
| 152 | 66 | Female | 85% | CC | CT | CC | TTA |
| 153 | 53 | Female | 96% | CG | TT | CC | TT |
| 154 | 67 | Male | 95% | CG | TT | TT | TT |
| 155 | 17 | Male | 87% | CC | CT | CT | TT |
| 156 | 59 | Male | 85% | CC | CT | CC | TT |
| 157 | 48 | Male | 68% | CC | CT | CT | TT |
| 158 | 57 | Male | 84% | CC | CT | CT | TT |
| 159 | 38 | Male | 80% | CC | CT | CC | TT |
| 160 | 36 | Male | 94% | CG | CT | CT | TTA |
| 161 | 37 | Female | 89% | CG | CC | CC | TT |
| 162 | 51 | Female | 70% | CG | CC | CC | TTA |
| 163 | 20 | Male | 92% | CC | TT | CC | TT |
| 164 | 57 | Male | 83% | CC | CT | CT | TATA |
| 165 | 39 | Female | 96% | CC | CT | TT | TTA |
| 166 | 19 | Female | 83% | CG | TT | CT | TTA |
| 167 | 25 | Male | 93% | CG | CC | TT | TTA |
| 168 | 55 | Male | 95% | CG | CT | CC | TTA |
| 169 | 38 | Female | 86% | CC | CC | CC | TT |
| 170 | 37 | Male | 85% | CC | CT | CC | TT |
| 171 | 21 | Male | 84% | CG | CT | CT | TT |
| 172 | 47 | Male | 85% | CG | CC | CC | TTA |
| 173 | 52 | Male | 85% | CG | CC | CC | TT |
| 174 | 55 | Female | 85% | CC | CC | TT | TTA |
| 175 | 29 | Male | 95% | CC | TT | CT | TT |
| 176 | 51 | Female | 95% | CG | TT | CT | TT |

Genotyping: DNA was extracted with the DNEASY® Blood & Tissue Kit (QIAGEN, Hilden, Germany) and samples were genotyped using TaqMan SNP genotyping assays for PNPLA3 (rs738409), MBOAT7 (rs641738), TM6SF2 (rs58542926), GCKR (rs780094), and TCF7L2 (rs7903146) (Thermo Fisher Scientific, San Jose, CA) (Table 2). HSD17B13 (rs72613567) genotyping primers were customized according to a previously published study (Yang et al. Hepatology. 70(1):231-40, 2019). Amplification and genotype clustering was performed using a STEPONEPLUS™ system (Applied Biosystems, Foster City, CA).

TABLE 2
Taqman ® SNP Genotyping Assay IDs used in this study.

| Target gene | SNP ID | Assay ID | Company |
|---|---|---|---|
| PNPLA3 | rs738409 | C_7241_10 | Life Technologies |
| MBOAT7 | rs641738 | C_8716820_10 | Life Technologies |
| TM6SF2 | rs58542926 | C_89463510_10 | Life Technologies |
| HSD17B13 | rs72613567 | ANAAH74 | Life Technologies |
| GCKR | rs780094 | C_2862873_10 | Life Technologies |
| TCF7L2 | rs7903146 | C_29347861_10 | Life Technologies |

Sanger sequencing: DNA extraction was performed with the KAPA Express Extract DNA Extraction Kit (Kapa Biosystems, London, UK). Polymerase chain reaction (PCR) amplification was conducted with the KOD ONE PCR Master Mix (Toyobo, Osaka, Japan) using the forward primer: 5′-CCA ACA ACC CTT GGT CCT GT-3′ (SEQ ID NO: 1) and reverse primer: 5′-GGG TAG CCT GGA AAT AGG GC-3′ (SEQ ID NO: 2) for PNPLA3. PCR products were then purified using the ExoSAP-IT Express PCR cleanup kit (Applied Biosystems, Foster City, CA) and sequenced at the Genomics Research Core at the University of Pittsburgh, Pennsylvania.

Hepatocyte isolation: Primary human hepatocytes were isolated using a three-step collagenase digestion technique as previously described (Gramignoli et al. 2012). Briefly, cell isolation was initiated by perfusion with pre-warmed (to 37° C.) calcium-free HBSS supplemented with 0.5 mM EGTA and collagenase solution (VitaCyte, Indianapolis, IN), until the tissue was fully digested. The digested liver was cooled with ice-cold Leibovitz's L-15 medium and strained through progressively smaller stainless-steel sieves with a final filtration through a 100 μm mesh. The final crude cell suspension was centrifuged twice, and the post-digest medium aspirated. Cell viability was assessed after isolation using trypan blue exclusion, and only cell preparations with a viability greater than 80% were cryopreserved. Single vials of cryopreserved hepatocytes were obtained from In Vitro ADMET Laboratories (Malden, MA) and Novabiosis (Durham, NC).

Production of High Titer Lentivirus: Vector cloning, vector sequencing and production of high-titer lentivirus was performed by Vectorbuilder (Chicago, IL). Briefly, lentiviruses were produced by co-transfecting 293T cells with the expression vectors pLV[Exp]-CMV>EGFP or pLV[Exp]-CMV>hGPX4 (transcript variant NM_001039848.4 modified to lack a poly-A sequence (so that the protein stays in the cytoplasm and does not get imported into the mitochondria), and including a SECIS element for incorporation of selenium, https://en.vectorbuilder.com/vector/VB210621-1038rsv.html AGTCCTGACTACGGCCTCCGGGCCCTTTGTCCCCGCTAGCGGCGCTCGGGGTGGGGGAGCCAG GAGGGGCGGGAGACGGGCGGGTATGGGCCGCGCGGGCGCAGGCTCCCCCGGGCGCCGCAGGC AGCGGTGCCAGAGCCGGGGCAGGCGGCGGCCGCGAGCCCCTCGGCGGCGGAAGGCCCCAGCG TGCAGGCGCAGGAGGGCGCGGCGCCGGCGGAAGAAGCCCTGTCCCCGCAGCTTGCGACCGGA GATCCACGAATGTCCCAAGTCCCAGGACCCGTGCGCGTCCCGGGACGACTGGCGCTGTGCGCG CTCCATGCACGAGTTTTCCGCCAAGGACATCGACGGGCACATGGTTAACCTGGACAAGTACCG GGGCTTCGTGTGCATCGTCACCAACGTGGCCTCCCAGTGAGGCAAGACCGAAGTAAACTACAC TCAGCTCGTCGACCTGCACGCCCGATACGCTGAGTGTGGTTTGCGGATCCTGGCCTTCCCGTGT AACCAGTTCGGGAAGCAGGAGCCAGGGAGTAACGAAGAGATCAAAGAGTTCGCCGCGGGCTA CAACGTCAAATTCGATATGTTCAGCAAGATCTGCGTGAACGGGGACGACGCCCACCCGCTGTG GAAGTGGATGAAGATCCAACCCAAGGGCAAGGGCATCCTGGGAAATGCCATCAAGTGGAACT TCACCAAGTTCCTCATCGACAAGAACGGCTGCGTGGTGAAGCGCTACGGACCCATGGAGGAGC CCCTGGTGATAGAGAAGGACCTGCCCCACTATTTCTAGCTCCACAAGTGTGTGGCCCCGCCCGA GCCCCTGCCCACGCCCTTGGAGCCTTCCACCGGCACTCATGACGGCCTGCCTGCAAACCTGCTG GTGGGGCAGACCCGAAAATCCAGCGTGCACCCCGCCGGAGGAAGGTCCCATGGCCTGCTGGGC TTGGCTCGGCGCCCCCACCCCTGGCTACCTTGTGGG [SEQ ID NO: 4]), envelope plasmid (pMD2. G) and packaging plasmids (pMDLg/pRRE and pRSV-Rev) using the FuGENE 6 transfection reagent (Promega Corporation, Madison, WI, USA). Lentivirus-containing supernatants were harvested and clarified by centrifugation, and then sterile-filtered through a 0.45 mM filter. Virus was then concentrated using a 100 KDa centrifugal filter unit (Amicon Ultra-15, Merck Kenilworth, NJ), before being stored at −80° C. Each lentivirus yielded>1×10⁹ viral particles.

Cell culture: Primary human hepatocytes were cultured in Hepatocyte Culture Medium (Lonza, Walkersville, MD) on type I rat tail collagen-coated plates (Corning, Corning, NY) at 37° C. in 5% CO₂. Only cell preparations with a post-thaw viability greater than 80% were used. Suspension culture of primary human hepatocytes was performed on ultra-low attachment plates (ThermoFisher Scientific, Waltham, MA) on an orbital shaker.

To create an environment of ferroptosis in primary human hepatocytes, 50 μM (5α,8α)-8-(1,1-dimethylethyl)-3-methyl-1,2-dioxaspiro[4.5]decane-3-ethanol (FINO2, Cayman Chemical, Ann Arbor, MI) dissolved in DMSO was added to the medium. To evaluate the protective effects of deferoxamine, primary human hepatocytes were treated with 100 μM deferoxamine (DFO, Sigma-Aldrich, Saint-Louis, MO) for 24 hours in suspension. To evaluate the protective effects of GSH, primary human hepatocytes on collagen-coated plates were treated with 10 μM reduced L-glutathione (GSH, G4251, Sigma-Aldrich, Saint-Louis, MO) for 48 hours.

A hepatocellular cell line was obtained from ATCC and maintained in DMEM (HepG2, GIBCO, Life Technologies, Carlsbad, CA, USA) supplemented with 10% HyClone fetal bovine serum (ThermoFisher Scientific, Waltham, MA) and 1% penicillin/streptomycin (ThermoFisher Scientific, Waltham, MA), and kept at 37° C. in 5% CO₂. For ferroptosis induction, 20 μM of (5α,8α)-8-(1,1-dimethylethyl)-3-methyl-1,2-dioxaspiro[4.5]decane-3-ethanol (FINO2, Cayman Chemical, Ann Arbor, MI) dissolved in DMSO was added to the medium. Transduction was performed with pLV[Exp]-CMV>EGFP or pLV[Exp]-CMV>hGPX4 particles at an MOI of 20 and in the presence of polybrene (8 μg/ml). GFP and GPX4 expression were evaluated 48 hours after transduction. Quality control tests included viability, sterility (with CASO-Bouillon, Heipha), and mycoplasma testing (Lonza, Walkersville, MD).

Cell Viability Measurements: Cell viability was assessed in 96-well format using Alamar Blue (Invitrogen, Carlsbad, CA) absorbance (570 nm/600 nm) measured on a synergy HTX microplate reader (Biotek, Winooski, VT) 24 hours after ferroptosis induction. Post thaw viability of primary human hepatocytes was evaluated by Trypan blue dye exclusion counting using a Countess 3 automated cell counter (Invitrogen, Carlsbad, CA).

Analysis of Lipid Peroxidation: A oxidation-sensitive probe C11-BODIPY (581/591) (Invitrogen, Carlsbad, CA) was used to evaluate lipid peroxidation under a fluorescence microscope. Untreated hepatocytes were prepared using a cytocentrifuge, incubated for 30 min with 10 μM C11-BODIPY, and washed with PBS. Cultured cells were incubated with 10 μM C11-BODIPY dissolved in culture media for 30 min at 37° C. and washed with PBS. The fluorescent signal was evaluated 3 hours after induction of ferroptosis.

Malondialdehyde (MDA) is a natural biproduct of lipid peroxidation and MDA quantification is used as marker for lipid peroxidation. The MDA content in primary human hepatocytes was quantified using a commercially available kit from Sigma-Aldrich (Sigma-Aldrich, Saint-Louis, MO). Cryopreserved primary human hepatocytes were pelleted and analyzed directly or were cultured for 3 hours in suspension before the analysis. Cell lysates were deproteinized and thiobarbituric acid (TBA) was added to generate an MDA-TBA adduct. The MDA-TBA adduct was quantified using fluorometric detection (ex/em=430 nm/590 nm).

Iron assay: The iron concentration in cell lysates was assessed using an Iron Assay Kit (#ab83366, Abcam, Cambridge, UK) according to the manufacturer's instructions.

Transmission electron microscopy: Human hepatocytes in suspension were briefly centrifugated and washed with PBS solution. Samples were then fixed with 2.5% glutaraldehyde over night at 4° C. Fixed samples were processed by the Center for Biologic Imaging at the University of Pittsburgh, and treated with 1% osmium tetroxide and 1% potassium ferricyanide for 1 hour at room temperature. Samples were washed with PBS and dehydrated in a graded series of ethanol solution (30%, 50%, 70%, and 90% ethanol; 10 minutes each) and three 15-minute incubations in fresh 100% ethanol. Infiltration/embedding was done with three 1-hour incubations of epon. The last change of epon was removed and beam capsules filled with resin were inverted over relevant areas of the monolayers. The resin was allowed to polymerize overnight at 37° C. and then for 48 hours at 60° C. Beam capsules and underlying cells were detached from the bottom of the 6-well plate and sectioned. Image acquisition was done using either a JEM-1011 or a JEM-1400Plus transmission electron microscopes (Jeol, Peabody, MA) at 80 kV fitted with a side mount AMT 2k digital camera (Advanced Microscopy Techniques, Danvers, MA). Mitochondrial shape was evaluated as published previously using ImageJ (Merrill et al. in Techniques to Investigate Mitochondrial Function in Neurons, 123:31-48. Neuromethods. Springer New York, 2017).

Quantitative Real Time PCR: Total cellular RNA was isolated using RNeasy Mini kit (QIAGEN, Hilden, Germany) and reverse transcribed using SUPERSCRIPT™ III (Invitrogen, Carlsbad, CA) following the manufacturers' instructions. qPCR was performed with a STEPONEPLUS™ system (Applied Biosystems, Foster City, CA) using TAQMAN® Fast Advanced Master Mix (Life Technologies, Waltham, MA) (Table 3). Relative gene expression was normalized to β-actin (ACTB) mRNA. Relative expression was calculated using the ΔΔCT method.

TABLE 3
TAQMAN ® Primers used in this study.

| Target gene | Assay ID | Company |
|---|---|---|
| FADS2 | Hs00927433_m1 | Life Technologies |
| ACSL4 | Hs00244871_m1 | Life Technologies |
| SCD | Hs01682761_m1 | Life Technologies |
| GPX4 | Hs00989766_g1 | Life Technologies |
| HSPB1 | Hs00356629_g1 | Life Technologies |
| TRF1 | Hs00951083_m1 | Life Technologies |
| FPN | Hs00205888_m1 | Life Technologies |
| NCOA4 | Hs00428331_g1 | Life Technologies |
| ATG5 | Hs00169468_m1 | Life Technologies |
| ACTB | Hs01060665_g1 | Life Technologies |

Western Blot: Cells were trypsinized, pelleted, and washed with PBS. Lysis was performed using RIPA buffer (Sigma-Aldrich, Saint Louis, Missouri) and 1× HALT™ Protease and Phosphatase Inhibitor Cocktail (Thermo Fisher Scientific, Waltham, MA) for 30 min at 4° C., followed by centrifuging at 13,000 g for 10 min at 4° C. Protein concentrations in the supernatant were determined by comparison with a known concentration of bovine serum albumin using a Pierce BCA Protein Assay Kit (ThermoFisher Scientific, Waltham, MA). 30 μg of lysate were loaded per well into a 10% Mini-PROTEAN TGX™ gel (BioRad, Hercules, CA). Proteins were transferred onto a PVDF Transfer Membrane (Thermo Fisher Scientific, Waltham, MA). Membranes were incubated with a primary antibody solution overnight and then washed before incubation in a secondary antibody solution for 1 hour. Target antigens were finally detected using SUPERSIGNAL™ West Pico PLUS Chemiluminescent Substrate (Thermo Fisher Scientific, Waltham, MA). Images were scanned and analyzed using ImageJ software. The antibodies used are listed in Table 2.

TABLE 4
Antibodies and dilutions used for western
blots and immunohistochemistry.

| Target protein | Catalog number | Company | Dilution | Host |
|---|---|---|---|---|
| anti-ACSL4 | ab124805 | Abcam | 1:5000 | Rabbit |
| anti-Ferritin | ab75973 | Abcam | 1:2000 | Rabbit |
| anti-GAPDH | 600041IG150UL | Proteintech | 1:10000 | Mouse |
| anti-NFEFL2 | ab62352 | Abcam | 1:250 | Rabbit |

Immunostaining: Human liver tissue was fixed in 4% paraformaldehyde for 12 hours and 70% ethanol overnight at 4° C., and then embedded in paraffin. Cut sections (5-7 microns) were mounted on glass slides for immunofluorescence and immunohistochemistry. Each sample was first stained with hematoxylin and eosin for histological examination. Slides were deparaffinized with xylenes and dehydrated with ethanol. Antigen unmasking was performed by boiling in 10 mM citrate buffer, pH 6.0. Slides were then blocked 1 hour with 10% donkey/goat serum and then incubated overnight at 4° C. with a primary antibody listed in the Table 2, followed by a secondary antibody for 1 hour at room temperature. Sections were covered with DAPI-containing mounting media. For immunohistochemistry staining, slides were incubated in 3% hydrogen peroxide. Tissue sections were then blocked and incubated with the secondary biotinylated antibody corresponding to the animal species of the primary antibody (BA-1000; Vector Laboratories, Burlingame, CA) and then exposed to 3,30-diaminobenzidine (SK-4105; Vector Laboratories) to visualize the peroxidase activity. Counterstaining was performed using Richard-Allan Scientific Signature Series Hematoxylin (Thermo Scientific, Waltham, MA). All procedures followed the kit instructions. Images were captured using a Nikon Eclipse Ti microscope.

Patients' hepatic paraffin sections were analyzed by two different pathologists. Pathologic findings were assessed according to the Liver Cancer Study Group of Japan. Liver steatosis was graded based on the percentage of fat: grade 0 (healthy, <5%), grade 1 (mild, 5%-33%), grade 2 (moderate, 34%-66%), and grade 3 (severe, >66%). Inflammation and fibrosis were evaluated according to the New Inuyama Classification for histological assessment score for chronic hepatitis.

Quantification and Statistical Analysis: Data from at least three sets of samples were used for statistical analysis. All statistical analyses were performed using Prism 6.0 Software (GraphPad Software, La Jolla, CA). Data are expressed herein as mean±standard deviation (SD). For normally distributed unpaired samples, assuming unequal SD, Welch's t-test was performed. For paired samples with normal distribution, paired t-test was used. Two-tailed Fisher's exact test was used for the analysis of contingency tables. Values of p<0.05 were considered statistically significant.

### Example 2

### Prevalence of Single Nucleotide Polymorphisms in Living Donor Liver Transplantation

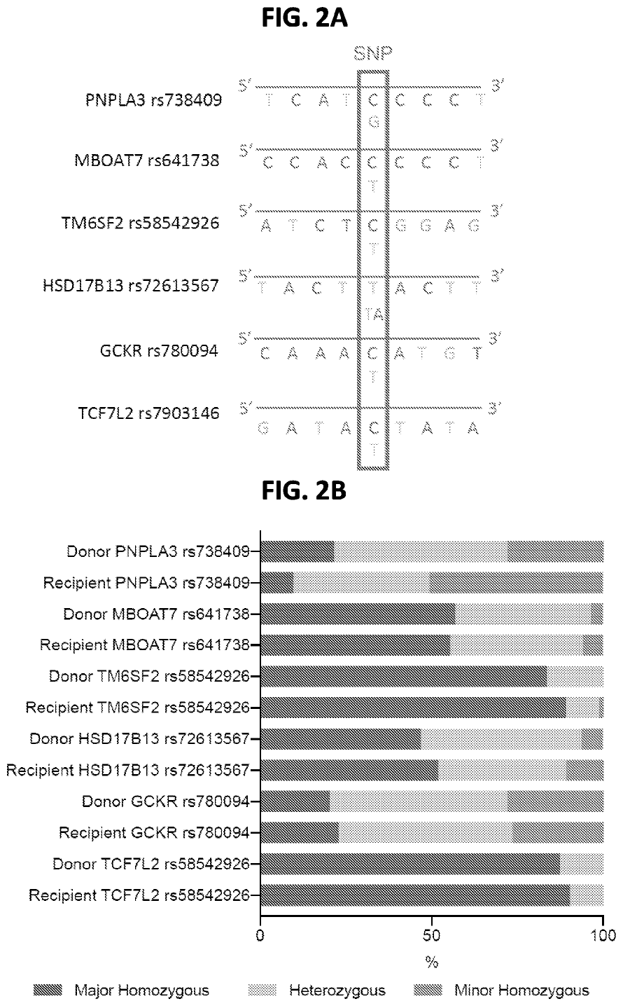

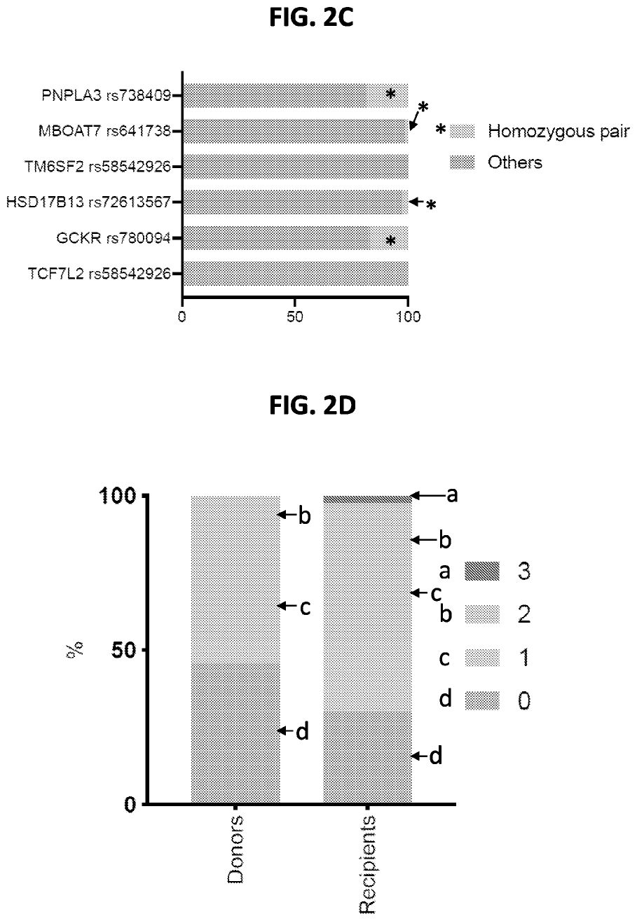

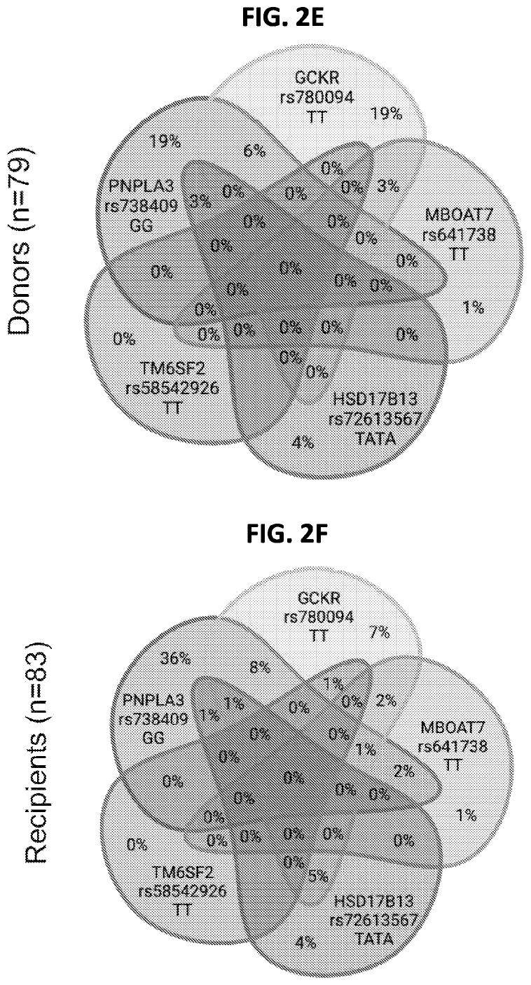

This example illustrates that single nucleotide polymorphisms are highly prevalent in recipients and donors of living donor liver transplantation. Eighty-three recipients and 79 donors underwent living donor liver transplantation for non-viral liver diseases (Laenec's cirrhosis n=45, NASH n=27, HCC n=21). Recipients and donors were genotyped for six different single nucleotide polymorphisms (SNPs) that have previously been correlated with hepatic diseases in genome-wide association studies (GWAS): PNPLA3 rs738409, MBOAT7 rs641738, TM6SF2 rs58542926, HSD17B13 rs72613567, GCKR rs780094 and TCF7L2 rs7903146 (FIG. 2A).

SNPs are highly prevalent in recipients and donors of living donor liver transplantation. Twenty-eight percent of recipients were homozygous for the PNPLA3 rs738409:G variant compared to 51% of donors (p=0.0014, Fisher's exact test). Other SNPs showed no difference among the recipient and donor population: MBOAT7rs641738 (6% vs 4%, p=0.75, Fisher's exact test), TM6SF2 rs58542926 (1% vs 0%, p>0.99, Fisher's exact test), HSD17B13 rs72613567 (11% vs 6%, p=0.31, Fisher's exact test) and GCKR rs780094 (27% vs 28%, p=0.88, Fisher's exact test). No donors or recipients were homozygous for the TCF7L2 rs58542926 variant (FIG. 2B). In approximately 10% of the cases, a PNPLA3 rs738409:G homozygous donor received a PNPLA3 rs738409:G homozygous liver graft (FIG. 2C). In approximately 10% of the cases, a GCKR rs780094:T homozygous donor received a GCKR rs780094:T homozygous liver graft (FIG. 2C). Homozygosity for two or more SNPs was insignificantly more common among the recipients (recipients 23% vs. donors 11%, p=0.06, Fisher's exact test) (FIG. 2D).

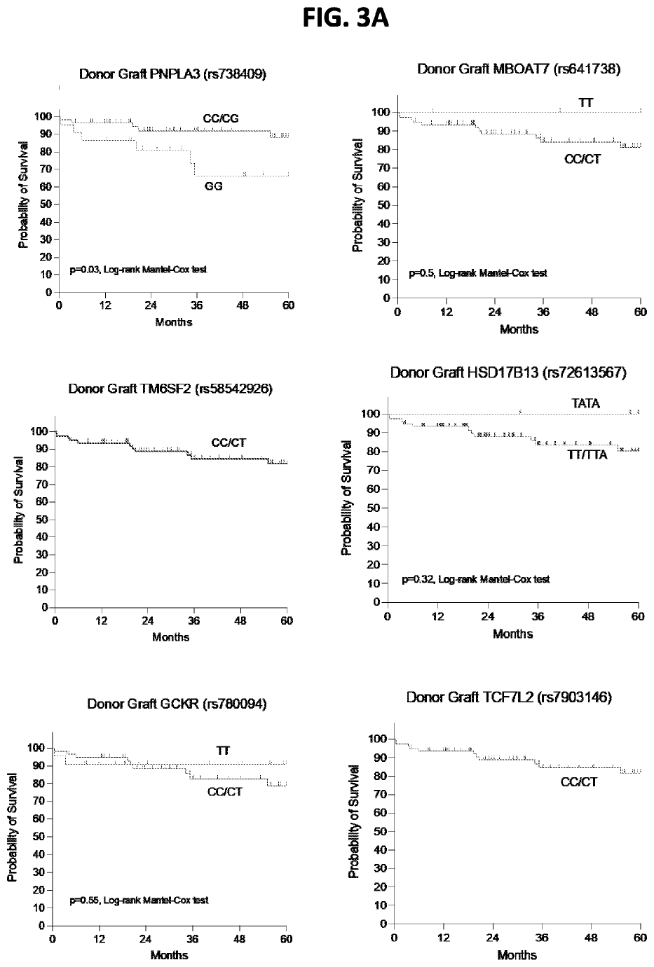

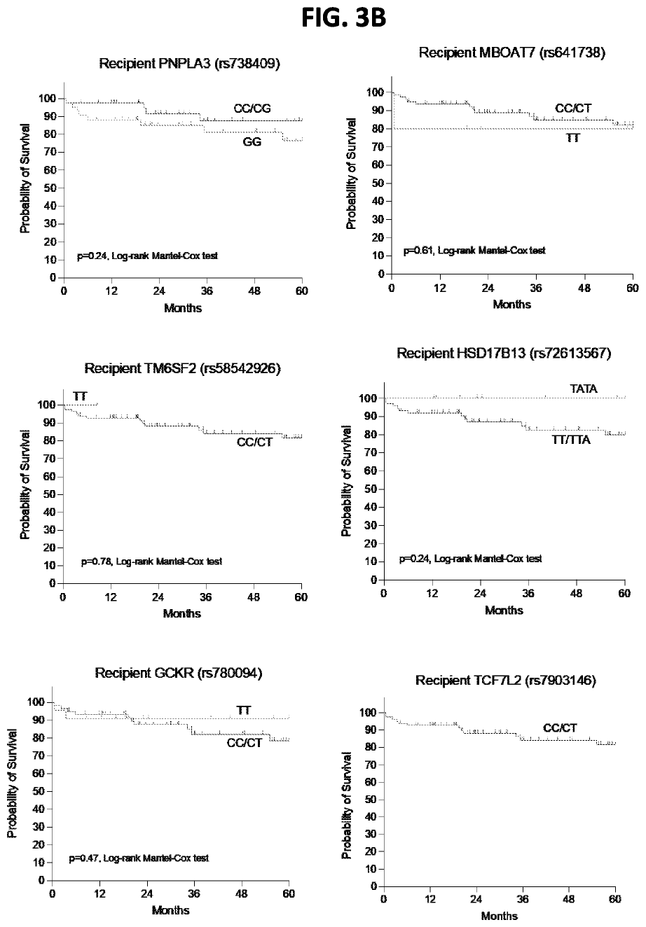

In this study, donor survival after donation was 100%. In total, 12 recipients died during the 5-year follow-up after transplantation. None of the recipients received a re-transplantation. To investigate the effect of SNPs in the donor graft, recipients of living donor liver transplantation were grouped according to the genotype of the donor graft. Recipients who received a donor graft homozygous for the PNPLA3 variant rs738409:G showed reduced postoperative 5-year survival (p=0.03, Log-rank Mantel-Cox test) (FIG. 3).

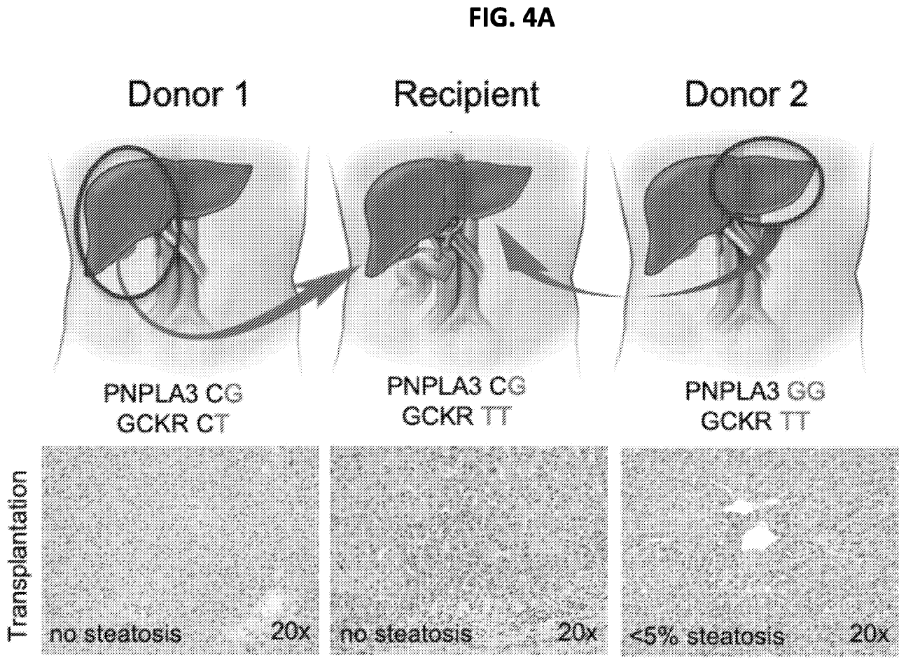

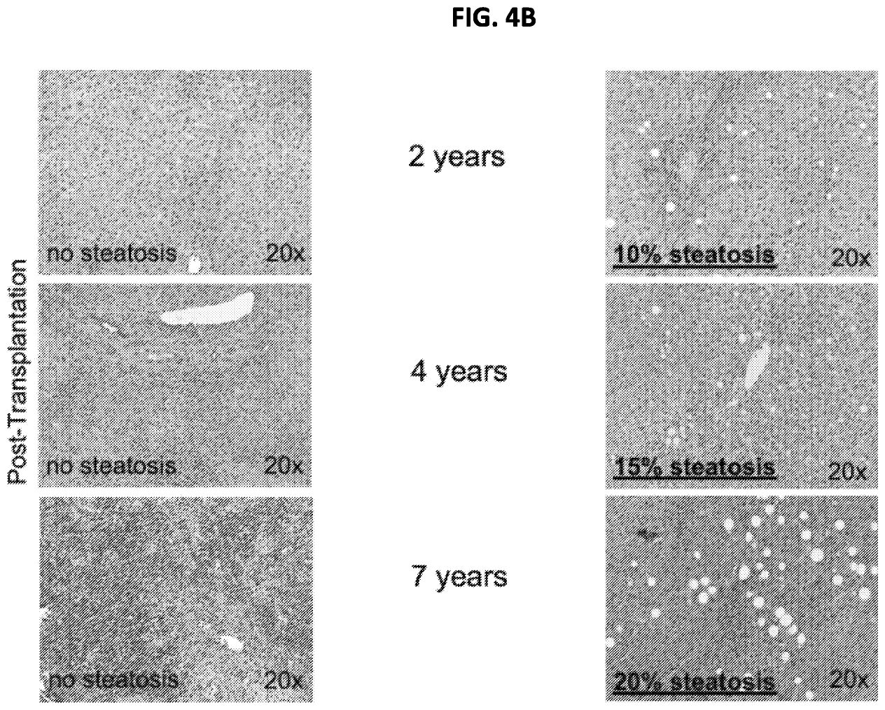

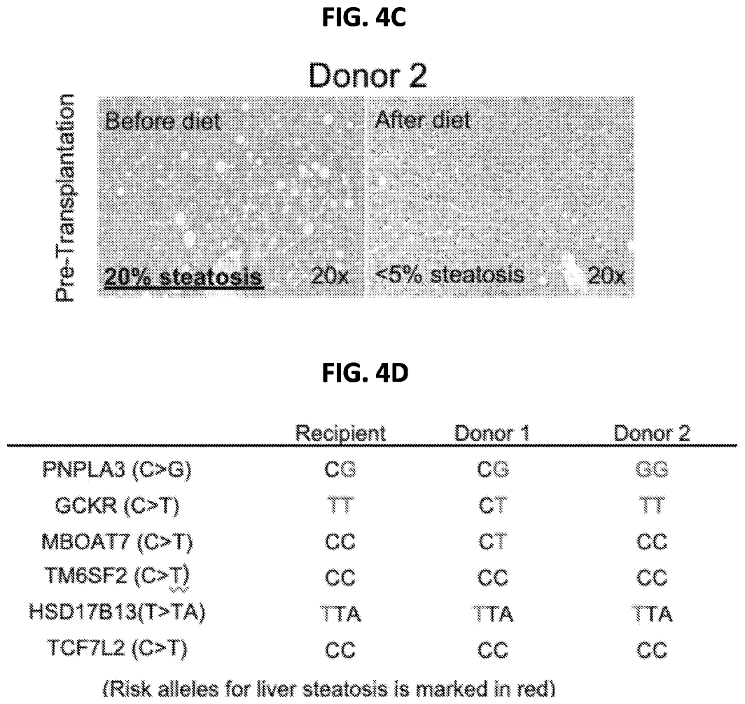

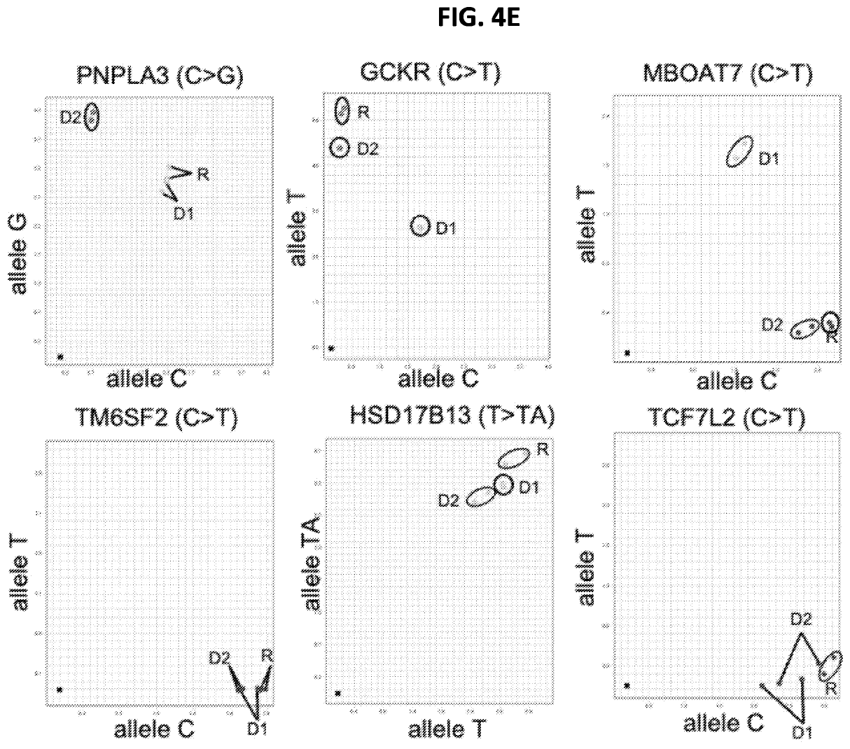

One patient received two donor grafts negative for hepatic steatosis before transplantation (<0.5% hepatic steatosis). This dual graft living donor liver transplantation case was published previously but had not yet been analyzed for the SNPs investigated in the present study (Soejima et al. 2008). The right lobe graft was heterozygous for the PNPLA3 rs738409:G variant and the GCKR rs780094 variant. The left lobe graft was homozygous for the PNPLA3 rs738409:G variant and the GCKR rs780094 variant. The left lobe graft developed postoperative steatosis whereas the right lobe graft did not develop postoperative steatosis, indicating a potential underlying genetic difference leading to hepatic steatosis (FIG. 4).

### Example 3

### Donor Hepatocytes Carrying the PNPLA3 rs738409:G Variant Show Metabolic Changes and Increased Membrane Turnover

This example illustrates that donor hepatocytes carrying the PNPLA3 rs738409:G variant show metabolic changes and increased membrane turnover. Ferroptosis is a form of programmed cell death different from other forms such as apoptosis or necrosis. Ferroptosis is dependent on iron causing lipid peroxidation and is associated with mitochondrial shrinkage (Dixon et al. Cell, 149:1060-72, 2012).

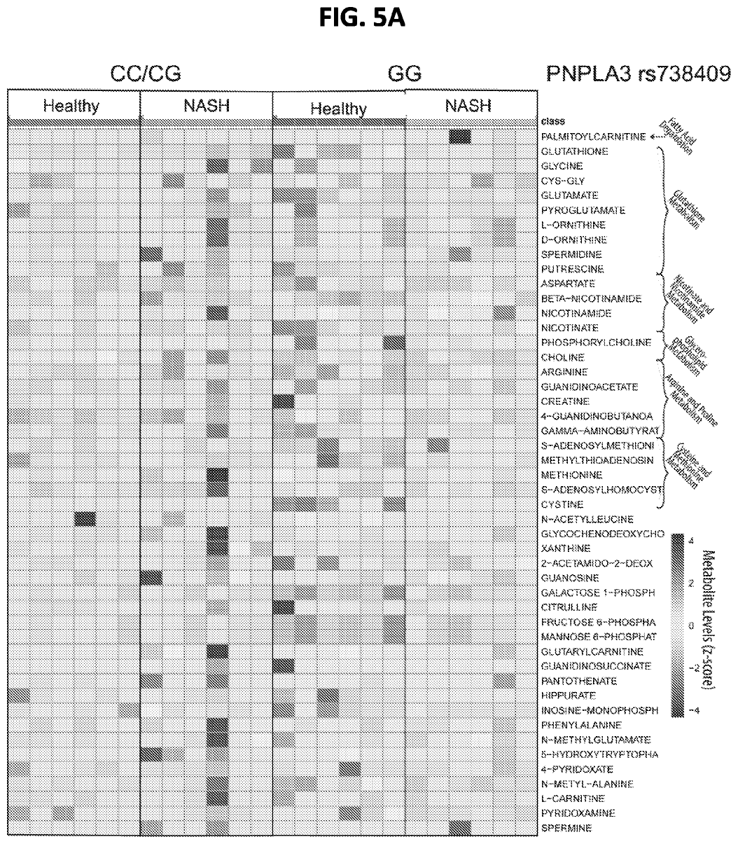

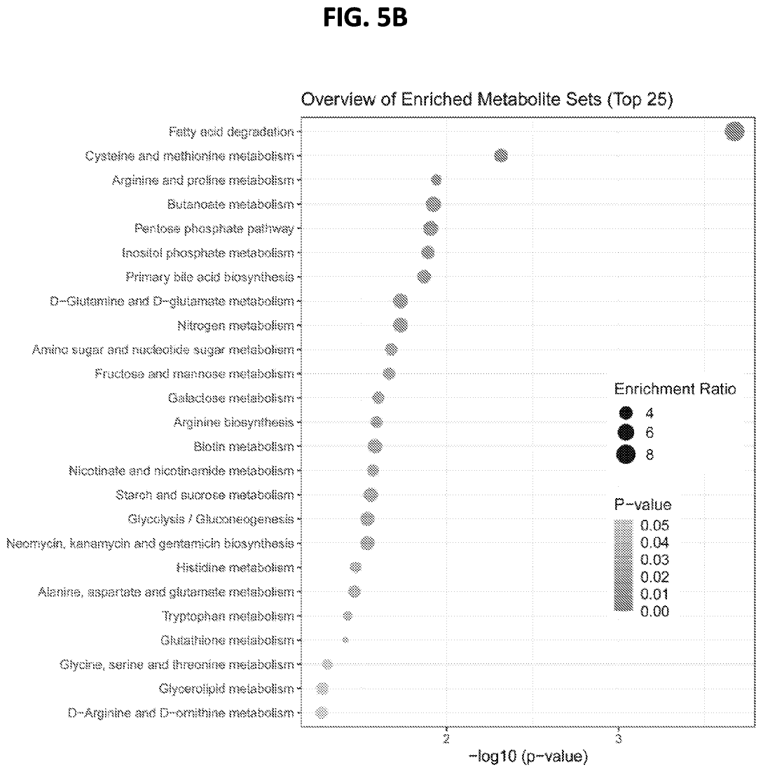

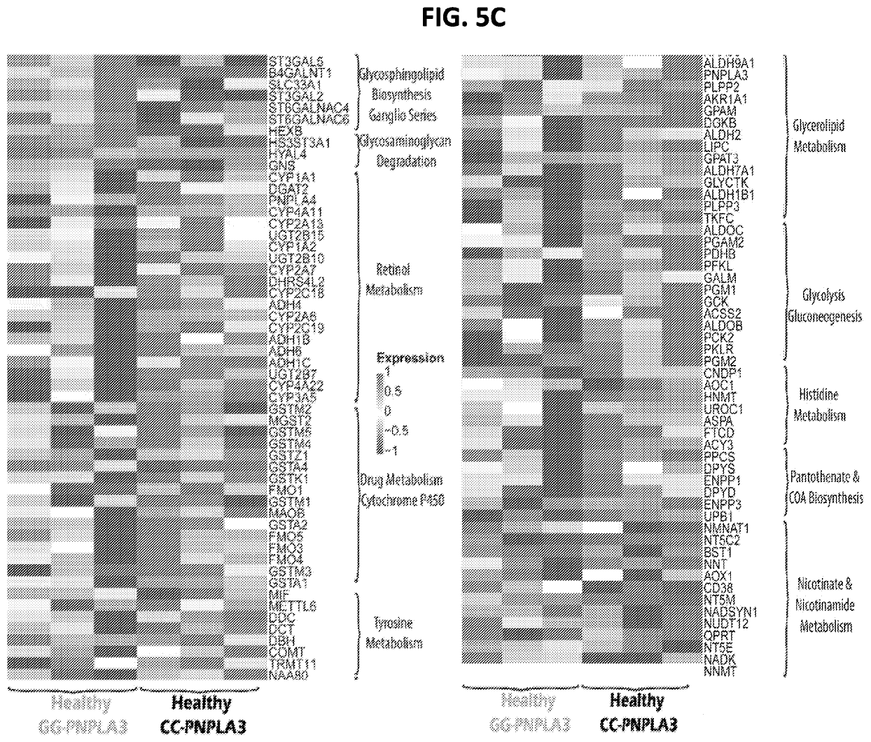

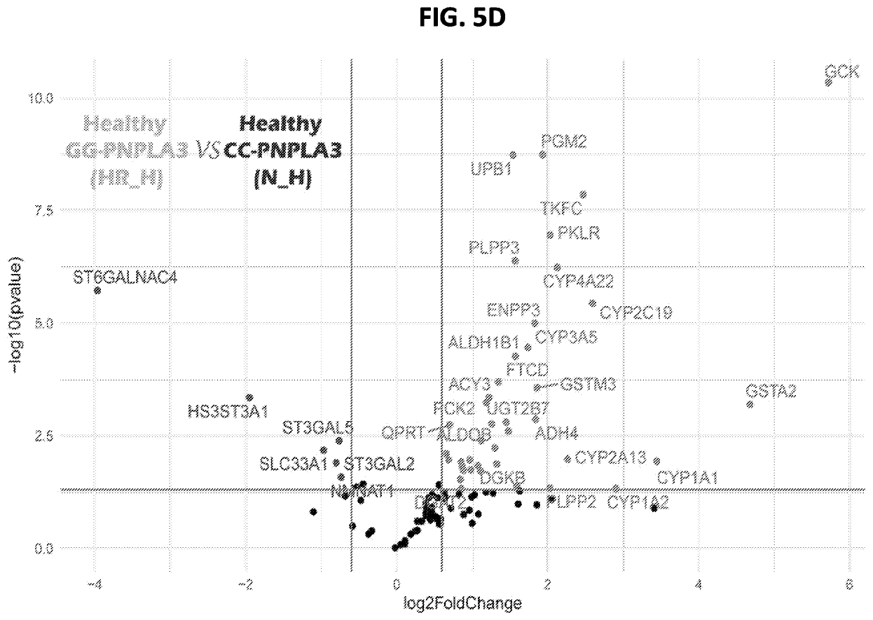

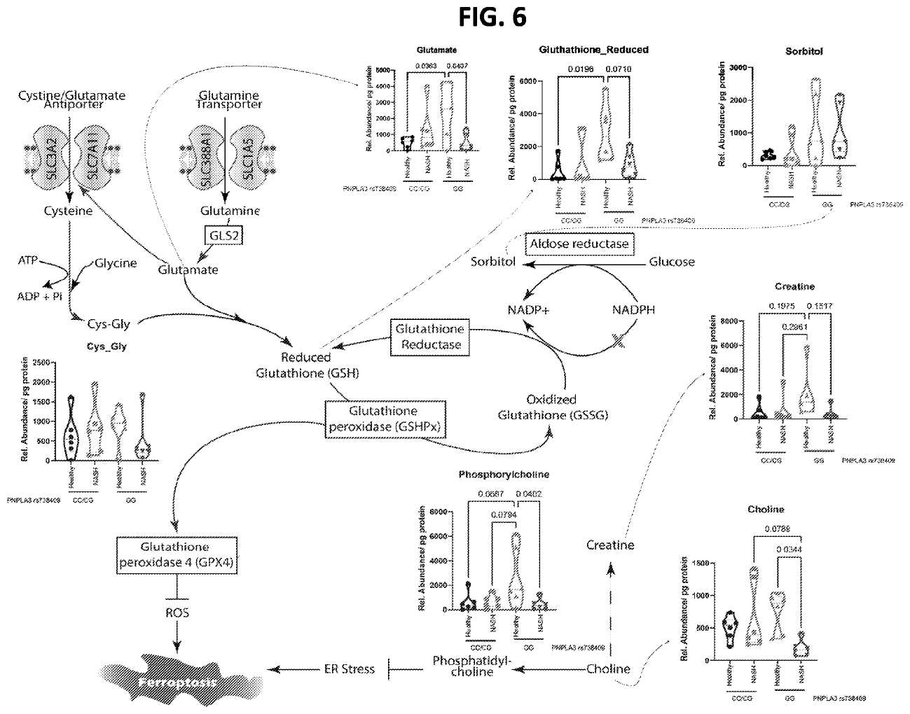

To investigate the underlying mechanisms of PNPLA3 rs738409:G causing reduced survival of recipients, hepatocytes of 176 donors were isolated and grouped according to their PNPLA3 rs738409 genotyping results. Transcriptomic analyses and sophisticated untargeted metabolic analyses were performed using donor hepatocytes with and without the PNPLA3 rs738409:G variant. Metabolomic analyses showed upregulation in the ferroptosis pathway (FIG. 5A-5B, FIG. 6). Transcriptomic analyses revealed increased transcription of enzymes involved in the biosynthesis of glycerolipids, indicating increased membrane turnover (FIG. 5C-5D, FIG. 6).

### Example 4

### Donor Hepatocytes with the PNPLA3 rs738409:G Variant Show Signs of Ferroptosis

This example illustrates that donor hepatocytes with the PNPLA3 rs738409:G variant show signs of ferroptosis, including increased lipid droplets and lipid peroxidation, reduced expression of GPX4, and mitochondrial changes.

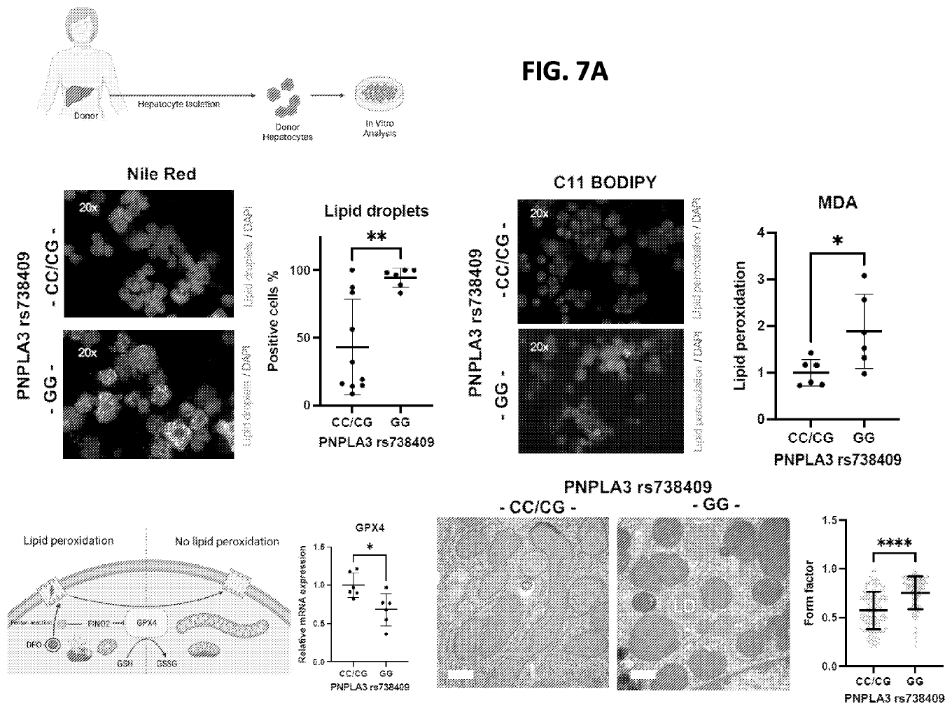

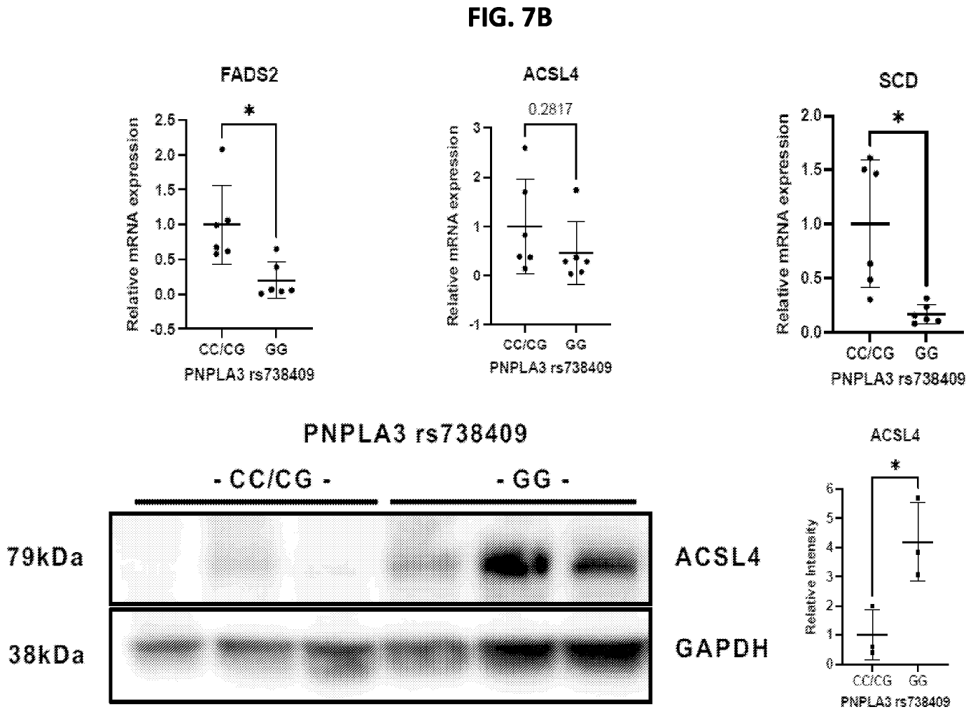

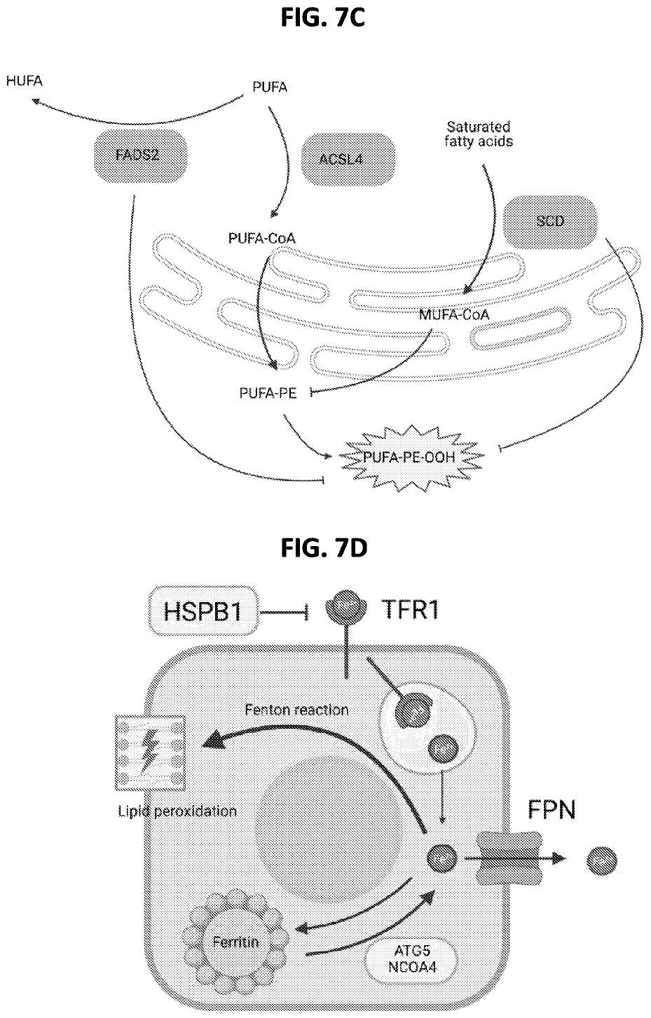

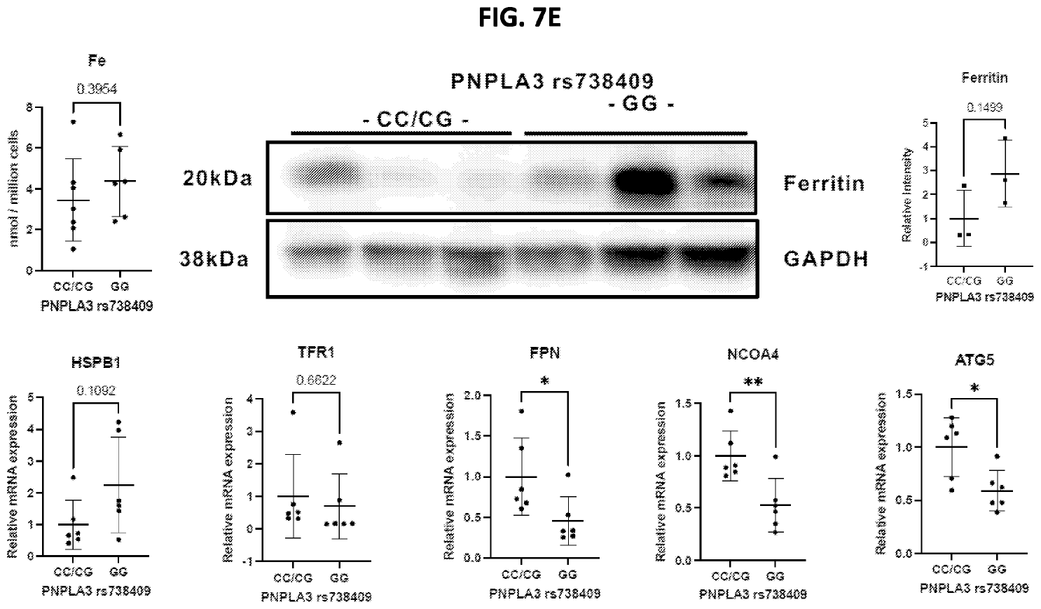

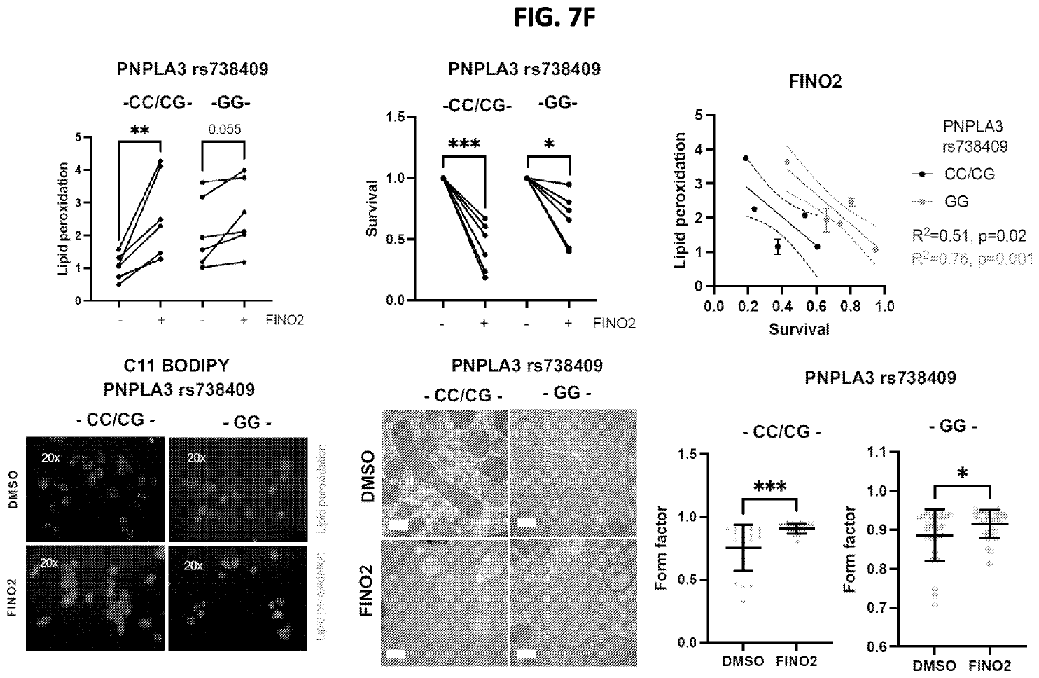

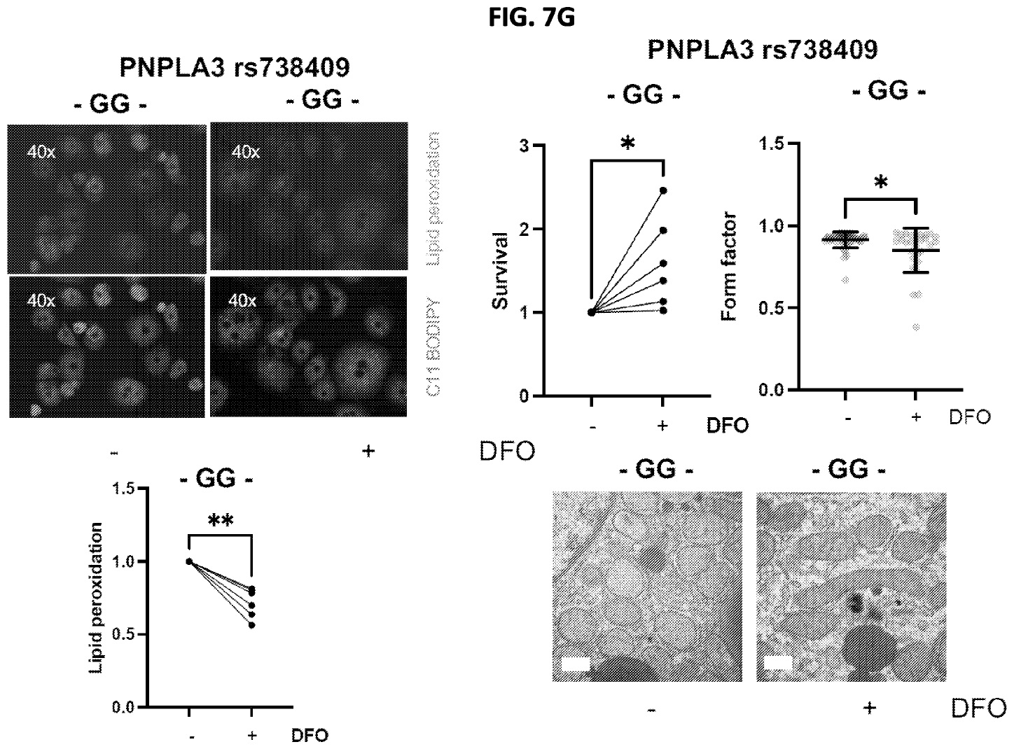

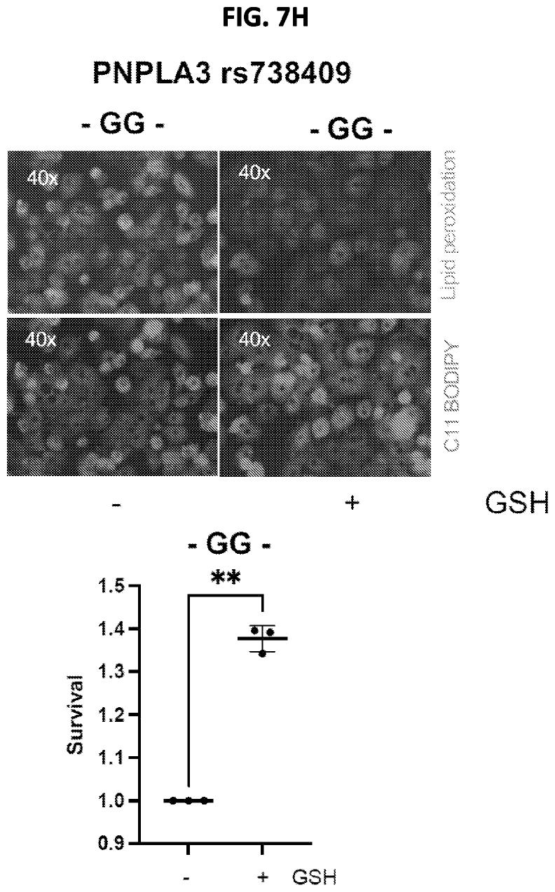

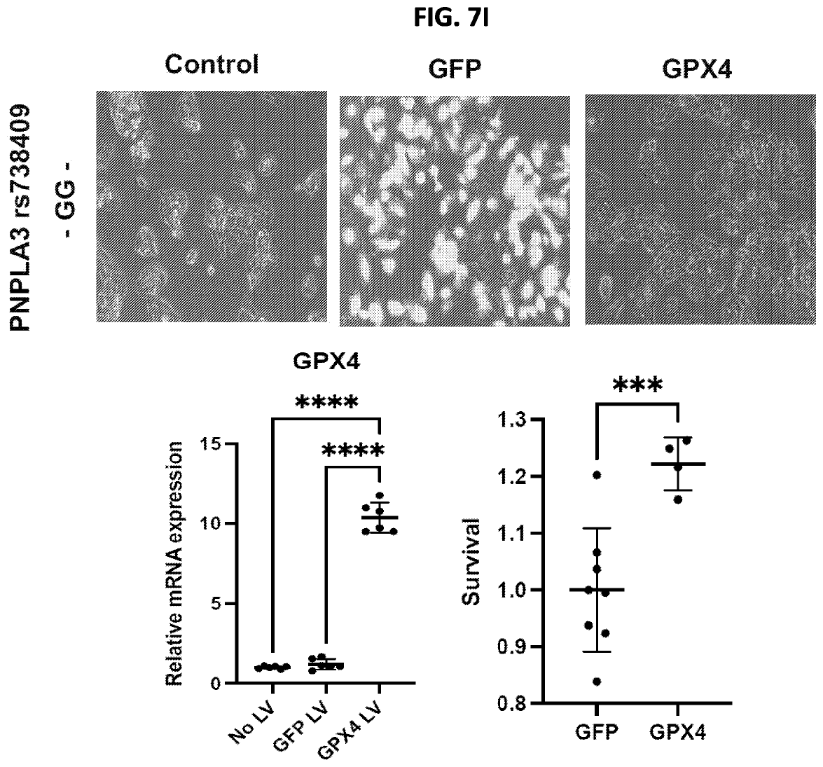

To corroborate the findings of increased hepatic steatosis, increased lipid peroxidation, and metabolic changes of ferroptosis with increased membrane turnover, donor hepatocytes were grouped according to their PNPLA3 rs738409 genotyping results. The PNPLA3 gene encodes for the patatin-like phospholipase domain-containing 3 protein that is located on the lipid droplets of hepatocytes and has a lipase activity preferentially for long-chain PUFAs (Li et al. J Clin Invest, 122, 4130-44, 2012; He et al. J Biol Chem, 285:6706-15, 2010). The PNPLA3 rs738409:G variant leads to a loss of function promoting the accumulation of triglycerides in lipid droplets of hepatocytes (He et al. J Biol Chem, 285:6706-15, 2010). Nearly all hepatocytes from donors carrying the PNPLA3 rs738409:G variant were positive for lipid droplets, more than hepatocytes not homozygous for the variant (**p=0.0012, Welch's t-test) (FIG. 7A). Lipid peroxidation levels were increased in hepatocytes positive for the PNPLA3 rs738409:G variant (*p=0.04, Welch's t-test) (FIG. 7A).

GPX4 is a key enzyme in the ferroptosis pathway and decreases lipid peroxidation. Reduced expression of GPX4 leads to increased ferroptosis susceptibility (Yang et al. Cell, 156:317-331, 2014). GPX4 expression was reduced among hepatocytes homozygous for the PNPLA3 rs738409:G variant (*p=0.02, Welch's t-test) (FIG. 7A). Because ferroptosis causes mitochondrial changes such as shrinkage (Dixon et al. Cell, 149:1060-72, 2012), mitochondrial shape was analyzed using transmission electron microscopy as published previously (Merrill et al. in Techniques to Investigate Mitochondrial Function in Neurons, 123:31-48. Neuromethods. Springer New York, 2017). Analyses revealed an increased number of spherical mitochondria (rather than rod shaped mitochondria), as commonly found in human hepatocytes and indicative of ferroptosis (****p<0.0001, Welch's t-test) (FIG. 7A).

### Example 5

### Alterations in Lipid Metabolism Increase Ferroptosis Susceptibility

This example illustrates that donor hepatocytes positive for the PNPLA3 rs738409:G variant exhibit reduced expression of FADS2 and SCD, and increased expression of ACSL4.

To investigate potential alterations in lipid metabolism that could explain ferroptosis, expression of metabolic enzymes associated with ferroptosis was investigated (Tesfay et al. Cancer Res, 79:5355-5366, 2019; Jiang et al. Theranostics, 7:3293-3305, 2017; Yuan et al. Biochem Biophys Res Commun, 478:1338-43, 2016). Hepatic triglycerides are especially enriched in PUFAs in carriers with the PNPLA3 rs738409:G variant (Luukkonen et al. J Hepatol, 64:1167-1175, 2016). PUFAs are highly susceptible to lipid peroxidation and are a key driver of ferroptosis (Yang et al. Proc Natl Acad Sci USA, 113:E4966-75, 2016).

FADS2 synthesizes highly unsaturated fatty acids from PUFAs, especially linoleic acid (LA) and alpha-linoleic acid (ALA) (Ge et al. J Invest Dermatol, 120:707-14, 2003), inhibiting ferroptosis (Jiang et al. Theranostics, 7:3293-3305, 2017). SCD contributes to the synthesis of monounsaturated fatty acids (MUFAs) using unsaturated fatty acids, leading to ferroptosis resistance (Tesfay et al. Cancer Res, 79:5355-5366, 2019). Expression of FADS2 (p=0.02, Welch's t-test) and SCD (p=0.02, Welch's t-test) was decreased in donor hepatocytes positive for the PNPLA3 rs738409:G variant (FIGS. 7B-7C).

ACSL4 is an enzyme that coverts free long-chain fatty acids into fatty acyl-CoA esters preferentially using PUFAs such as arachidonate. ACSL4 overexpression enhances ferroptosis sensitivity and drives ferroptosis (Yuan et al. Biochem Biophys Res Commun, 478:1338-43, 2016). ACSL4 expression was upregulated at the protein level in primary human hepatocytes homozygous for the PNPLA3 rs738409:G variant (*p=0.03, Welch's t-test) (FIGS. 7B-7C).

### Example 6

### Compensatory Changes in Iron Metabolism Reveal Protection Mechanisms from Ferroptosis

This example illustrates that genes involved in iron metabolism are dysregulated in hepatocytes positive for the PNPLA3 rs738409:G variant. Iron and the oxidation of iron are essential in the development of lipid peroxidation and ferroptosis (Dixon et al. Cell, 149:1060-72, 2012). Therefore, total iron content of donor hepatocytes positive for the PNPLA3 rs738409:G variant was investigated. These cells showed similar amounts of cellular iron content (p=0.4, Welch's t-test) (FIGS. 7D-7E). To investigate the cellular distribution of iron, ferritin expression was assessed. Ferritin is the major intracellular iron storage protein. Ferritin expression was insignificantly increased among donor hepatocytes carrying the PNPLA3 rs738409:G variant (p=0.15, Welch's t-test) (FIGS. 7D-7E). To further investigate iron distribution in the hepatocytes, expression levels of key genes involved in iron distribution and ferroptosis were assessed. Heat Shock Protein Family B Member 1 (HSPB1) is known to inhibit the transferrin receptor (TFR1) and negatively regulate ferroptosis (Sun et al. Oncogene, 34:5617-25, 2015). HSPB1 expression was insignificantly increased in donor hepatocytes carrying the PNPLA3 rs738409:G variant (p=0.11, Welch's t-test) (FIGS. 7D-7E). Ferroportin (FPN), which is involved in iron export, was significantly downregulated in PNPLA3 rs738409:G variant positive donor hepatocytes (p=0.04, Welch's t-test) (FIGS. 7D-7E). Nuclear receptor coactivator 4 (NCOA4) and Autophagy-Related Gene 5 (ATG5) are involved in ferritin degradation and downregulation of these genes can protect against ferroptosis (Mancias et al. Nature, 509:105-9, 2014; Hou et al. Autophagy, 12:1425-8, 2016) (FIGS. 7D-7E). NCOA4 (**p=0.008, Welch's t-test) and ATG5 (*p=0.02, Welch's t-test) were significantly downregulated in donor hepatocytes positive for the PNPLA3 rs738409:G variant (FIGS. 7D-7E).

### Example 7

### PNPLA3 rs738409:G Variant Positive Donor Hepatocytes Show Resilience Against Acute Induction of Ferroptosis

This example illustrates that, surprisingly, PNPLA3 rs738409:G variant positive donor hepatocytes showed reduced susceptibility when stressed with FINO2-induced lipid peroxidation. To test the effectiveness of the cellular iron distribution pattern of PNPLA3 rs738409:G variant-carrying donor hepatocytes on ferroptosis prevention, ferroptosis was induced in donor hepatocytes using FINO2, an inducer of iron oxidation (Gaschler et al. Nat Chem Biol, 14:507-515, 2018).

Donor hepatocytes cultured in the presence of FINO2 showed increased lipid peroxidation (**p=0.01, p=0.055, paired t-test) and a significant reduction in donor hepatocyte survival (***p=0.001, *p=0.01, paired t-test) (FIG. 7F). Lipid peroxidation and donor hepatocyte survival were negatively correlated (R²=0.51, p=0.02; R²=0.76 p=0.001) (FIG. 7F). Surprisingly, hepatocytes carrying the PNPLA3 rs738409:G variant showed reduced susceptibility when stressed with similar levels of lipid peroxidation by FINO2.

In the presence of FINO2, mitochondrial shape changed from a rod shape to a spherical shape (***p=0.0006, *p=0.05, Welch's t-test) with a more pronounced shape change in the controls negative for the PNPLA3 rs738409:G variant (FIG. 7F).

### Example 8

### Causal Ferroptosis Treatment Improves Cell Survival

This example illustrates that transduction of GPX4 into PNPLA3 rs738409:G variant-carrying hepatocytes induces GPX4 expression and increases cell survival. Three different approaches were employed to evaluate treatment options against ferroptosis for hepatocytes carrying the PNPLA3 rs738409:G variant: (1) a pharmacological approach using the approved iron chelator deferoxamine (DFO); (2) a dietary approach supplementing reduced L-Glutathione (GSH); and (3) a gene therapy approach forcing GPX4-expression.

Donor hepatocytes positive for the PNPLA3 rs738409:G variant showed reduced lipid peroxidation, increased survival, and improved mitochondrial shape (p=0.02, Welch's t-test) when treated with DFO in an environment of ferroptosis (FIG. 7G).

When GSH was supplemented, donor hepatocytes positive for the PNPLA3 rs738409:G variant showed decreased lipid peroxidation and increased survival (p=0.002, paired t-test) (FIG. 7H).

To evaluate the potential of a gene therapy forcing GPX4 expression, a PNPLA3 rs738409:G variant-carrying hepatocellular cell line was transduced. Transduction of GPX4 significantly induced GPX4 expression (****p<0.0001, Brown-Forsythe ANOVA test) and increased cell survival (p=0.0006, Welch's t-test) (FIG. 7I).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of promoting survival of a donor liver in a recipient subject, wherein the donor liver is homozygous for rs738409:G mutation in the patatin-like phospholipase domain-containing protein 3 (PNPLA3) gene, the method comprising administering to the subject a therapeutically effective amount of:
(a) a nucleic acid molecule encoding glutathione peroxidase 4 (GPX4); or
(b) glutathione (GSH);
thereby promoting survival of the donor liver in the recipient subject.

2. A method of promoting survival of a donor liver in a recipient subject, wherein the donor liver is homozygous for rs738409:G mutation in the PNPLA3 gene, the method comprising administering to the donor liver a therapeutically effective amount of a nucleic acid molecule encoding GPX4, thereby promoting survival of the donor liver in the recipient subject.

3. The method of claim 1, wherein the donor liver is from a donor, and wherein the method further comprises detecting the rs738409:G mutation in the PNPLA3 gene in a sample from the donor.

4. The method of claim 3, wherein the sample from the donor is a blood or tissue sample.

5. The method of claim 4, wherein the tissue sample is a liver sample.

6. The method of claim 1, wherein the subject is the recipient of a cadaveric liver transplant.

7. The method of claim 1, wherein the subject is the recipient of a liver transplant from a living donor.

8. The method of claim 1, comprising administering to the subject the therapeutically effective amount of the nucleic acid molecule encoding GPX4.

9. The method of claim 1, comprising administering to the donor liver the therapeutically effective amount of the nucleic acid molecule encoding GPX4 in an ex vivo perfusion system.

10. The method of claim 1, wherein the nucleic acid molecule encoding GPX4 is an mRNA.

11. The method of claim 10, wherein the mRNA is administered to the subject using lipid nanoparticles, using polymeric nanoparticles, as a conjugate to GalNAc, as an mRNA modified by base linker sugars, using a degradable polymer, as an mRNA-Lipoplex, or as mRNA cargo of PEG-10.

12. The method of claim 1, comprising administering to the subject a therapeutically effective amount of a viral vector comprising the nucleic acid molecule encoding GPX4.

13. The method of claim 12, wherein the viral vector is a lentiviral vector.

14. The method of claim 12 wherein the viral vector is an adeno-associated virus (AAV) vector.

15. The method of claim 14, wherein the AAV vector is an AAV1, AAV2, AAV3, AAV4, AAV5, AAV6, AAV7, AAV9, AAV10, AAV11, or AAV12 vector, or a hybrid of two or more AAV serotypes.

16. The method of claim 1, comprising administering to the subject the therapeutically effective amount of GSH.

17. The method of claim 1, further comprising administering to the subject a therapeutically effective amount of deferoxamine, selenium, vitamin E (alpha-tocopherol), CoQ10, or a combination thereof.

18. The method of claim 1, wherein the method inhibits lipid peroxidation and/or mitochondrial shrinkage in donor liver cells, and/or increases survival of donor liver cells in the subject.

19-20. (canceled)
